(19) United States
(12) Patent Application Publication
(10) Pub. No.: US 2023/0416196 A1
(43) Pub. Date: Dec. 28, 2023

(54) DIMER OF BIGUANIDINES AND THEIR THERAPEUTIC USES

(71) Applicants: INSTITUT CURIE, PARIS (FR); INSERM (INSTITUT NATIONAL DE LA SANTE ET DE LA RECHERCHE MEDICALE), PARIS (FR); CENTRE NATIONAL DE LA RECHERCHE SCIENTIFIQUE, PARIS (FR); PARIS SCIENCES ET LETTRES, PARIS (FR)

(72) Inventors: RAPHAËL RODRIGUEZ, IVRY-SUR-SEINE (FR); ANTOINE VERSINI, ETERVILLE (FR); TATIANA CAÑEQUE, PARIS (FR); SEBASTIAN MÜLLER, IVRY-SUR-SEINE (FR); STÉPHANIE SOLIER, PARIS (FR); LEEROY BARON, SAINT-MANDE (FR)

(21) Appl. No.: 18/037,331

(22) PCT Filed: Nov. 18, 2021
(86) PCT No.: PCT/EP2021/082073

§ 371 Date: May 17, 2023

(30) Foreign Application Priority Data
Nov. 18, 2020 (EP) 20306402.7

(51) Int. Cl. C07C 279/26; A61P 35/00

(52) U.S. Cl. CPC C07C 279/265; A61P 35/00

(57) ABSTRACT

The present invention relates to compounds comprising two biguanidyl radicals that can be useful as anti-inflammatory agent, and also to new compounds comprising two biguanidyl radicals and their use as a drug, in particular for treating a cancer, a metabolic disease, a secondary mitochondrial disorder due to copper overload including Indian childhood cirrhosis, Wilson's disease and Idiopathic infantile copper toxicosis or due to iron overload, an infection by a virus such as a coronavirus or an influenza virus, a neurodegenerative disease or disorder and aging.

### FUNDING

The project leading to this application has received funding from the European Research Council (ERC) under the European Union's Horizon 2020 research and innovation program (grant agreement No [647973]), from Fondation Charles Defforey-Institut de France and Ligue Contre le Cancer.

### FIELD OF THE INVENTION

The present invention relates to the field of medicine, mainly as anti-inflammatory and anti-carcinogenic agent, but also other therapeutic indications. The present invention also relates to new compounds comprising two biguanidyl radicals.

### BACKGROUND OF THE INVENTION

A variety of derivatives of biguanide are used as pharmaceutical drugs. Most of them are used as antihyperglycemic agents, in particular used for the treatment of diabetes mellitus and prediabetes. The most widely used drug is metformin.

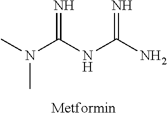

Derivatives of biguanide are also used as antimalarial drugs such as proguanil and chloproguanil and as disinfectants such as chlorhexidine, polyaminopropyl biguanine, polihaxanide, and alexidine.

More recently, metformin and derivatives thereof have been described for their use in the treatment of cancer (Safe et al, 2018, Biol Chem, 399, 321-335; WO2017/192602). More particularly, patent applications disclose derivatives of metformin (EP2522653, EP3222614, WO2013/022279, WO2014/123364, WO2015/160220, WO2016/025725 and WO2016/155679).

More recently, the inventors designed new compounds comprising biguanidyl radical, which are more potent than metformin for the treatment of cancer (WO 2019/233982).

However, there is still a strong need of new compounds and drugs for treating the disease more efficiently and for treating new emerging diseases.

Severe acute respiratory syndrome-coronavirus 2 (SARS-CoV-2) can induce a cytokine release syndrome (CRS) leading to the human respiratory illness coronavirus disease 2019 (COVID-19). CRS, resulting from the uncontrolled production of cytokines by inflammatory macrophages (MDM), has been identified as a central cause of death in COVID-19. There is as yet no effective strategy for the clinical management of CRS.

Analyses of bronchoalveolar fluids of COVID-19 patients revealed the prevalence of inflammatory macrophages that can lead to respiratory failure and death. Although it has been shown that epigenetic reprogramming regulates monocyte-to-macrophage transition during inflammation, mechanisms underlying the global expression of inflammatory cytokines are not fully understood. The membrane protein CD44 has been implicated in hematopoiesis and plays a role during inflammation of lung macrophages. Furthermore, the inventors have shown that CD44 mediates endocytosis of iron regulating the plasticity of cancer cells (Müller et al., 2020, Nature Chemistry, 12, 929-938).

### SUMMARY OF THE INVENTION

Here, the inventors report the discovery that CD44 mediates endocytosis of the d-block metals copper and iron through interactions with hyaluronates in inflammatory macrophages, thereby regulating metabolic and epigenetic plasticity underlying the expression of cytokines. They show that inflammatory human macrophages upregulate endocytosis of iron and copper. Copper is used in mitochondria to replenish the pool of NAD⁺, the enzymatic co-substrate required for the production of α-ketoglutarate (αKG) and acetyl-coenzyme A (acetyl-CoA), while αKG itself together with iron directly mediate oxidative demethylation of repressive histone marks and acetyl-CoA the concomitant acetylation status, thereby unlocking the expression of cytokines. Importantly, they have developed highly potent compounds comprising biguanidyl radicals that blocks the oxidation of NADH into NAD⁺ by chelating mitochondrial copper, inhibiting the production of αKG and the activation of macrophages, as well as blocking cell plasticity in cancer cells. This strategy provides solid basis for the treatment of inflammatory diseases including among others, septic shock and COVID-19.

The present study provides a unifying mechanism linking the prevalent role of CD44, hyaluronates and metals in the regulation of hematological cell plasticity involved in inflammation. It sheds light onto the functional relevance of high systemic levels of ferritin and the abundance of hyaluronate in the lungs of severe COVID-19 patients. It delivers new insights as to why diabetic and obese patients are more vulnerable to SARS-CoV-2 (dysregulated glucose metabolism, glucose being a precursor of αKG). Finally, it explains how the FDA-approved drug metformin exerts its beneficial activity in COVID-19 patients (although with a moderate potency in vitro compared to the compounds of the present invention). Then, the inventors demonstrate that a compound comprising two biguanidyl radicals can be useful as an anti-inflammatory agent. More particularly, as shown in the examples section, they are able to prevent the activation of macrophages, in particular with an efficacy of 1,000-fold over Metformin (see FIG. 3). More generally, the compound with two biguanidyl radicals are able to chelate copper by forming a complex. Without wishing to be bound by theory, it has been hypothesized that the presence of the two biguanidyl radicals in the compounds bound together by a linker leads to a favorable context to form a complex with copper, such a complex needing one copper and two biguanidyl radicals.

Accordingly, the present invention relates to a compound as defined above for use as anti-inflammatory agent. It further relates to new compounds comprising two biguanidyl radicals. It finally relates to alternative therapeutic uses of the compounds of the present invention, including blocking cell plasticity (cancer stem cell formation) in pancreatic ductal adenocarcinoma (PDAC).

Accordingly, the present invention relates to a compound of formula (I) for use as anti-inflammatory agent,

wherein the formula (I) is

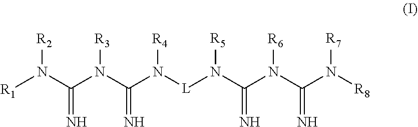

with
R₁, R₂, R₃, R₄, R₅, R₆, R₇ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;
or
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group, and R₁ and R₈ forming together a linker -L′-,
-L- and -L′-, if present, being independently a linear hydrocarbon chain of 8 to 16 carbons optionally interrupted by
   An oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy, or a pharmaceutically acceptable salt, stereoisomer, tautomer or solvate thereof.

Preferably, the compound is for use for the treatment of an inflammatory or autoimmune disease or disorder, especially for use for the treatment of a systemic inflammatory response syndrome, a cytokine release syndrome (CRS), an Adult Respiratory Distress Syndrome (ARDS), a Macrophage Activation Syndrome (MAS), an Alveolar inflammatory response, a paediatric multisystem inflammatory syndrome, a Hemophagocytic lymphohistiocytosis (HLH), systemic lupus erythematosus, a sepsis, in particular septic shock, Crohn's disease, ulcerative colitis, rheumatoid arthritis, or a hypercytokinemia; for use for the treatment of a cytokine release syndrome induced by a virus of Orthocoronavirinae subfamily such as Middle East respiratory syndrome-related coronavirus (MERS-CoV), β-CoV, Severe acute respiratory syndrome coronavirus (SARS-CoV), 3-CoV or Severe acute respiratory syndrome coronavirus 2 (SARS-CoV-2), P-CoV; or for use for the treatment of a disease selected from the group consisting of a metabolic disease, especially diabetes mellitus including type 1 and type 2 diabetes mellitus, insulin resistance, hyperglycemia, hyperinsulinemia, glucose intolerance, hypertension, NAFLD, NASH and obesity, polycystic ovary syndrome, metabolic syndrome, cardiovascular diseases including hypertension, atherosclerosis and arteriosclerosis, a mitochondrial dysfunction including a primary or a secondary mitochondrial dysfunction, especially a secondary mitochondrial disorder due to copper overload including Indian childhood cirrhosis, Wilson's disease and Idiopathic infantile copper toxicosis or due to iron overload including Hereditary Hemochromatosis, Juvenile Hemochromatosis, Neonatal iron storage disease, type I Tyrosinemia and Zellweger syndrome, and mental disorders including schizophrenia, anxiety disorders, mild cognitive disorder, depressive disorder, bipolar disorder, autism spectrum disorder and Fragile X syndrome, an infection by a virus such as a coronavirus or an influenza virus, a neurodegenerative disease or disorder and aging, preferably selected from the group consisting of diabetes mellitus including type 1 and type 2 diabetes mellitus, polycystic ovary syndrome, and glucose intolerance.

In an additional aspect, the present invention relates to a new compound of formula (I) wherein the formula (I) is

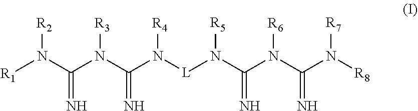

wherein
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;

and

1) R₁ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group; and

-L- being independently a linear hydrocarbon chain of 11 to 16 carbons optionally interrupted by
An oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

said linear hydrocarbon chain being optionally substituted by a group R or R′;

or

2) R₁ and R₈ forming together a linker -L′-; and

-L- and -L′- being independently a linear hydrocarbon chain of 4 to 16 carbons optionally interrupted by
a heteroatom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

said linear hydrocarbon chain being optionally substituted by a group R or R′;

or

3) the compound is selected in the group consisting of

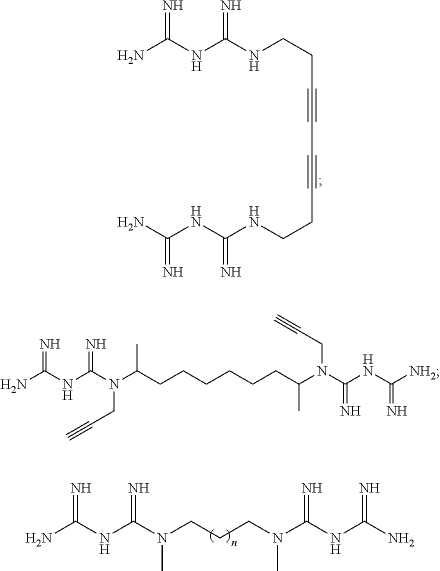

with n being an integer from 2-14 or from 4-14 or from 8-14 or from 8-12 or from 8-10; and

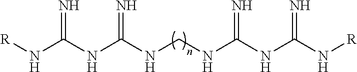

with n being an integer from 6-16, from 8-16 or from 8-12 or from 8-10 and R being an ethyl, a propyl or —CH2-C≡CH,

wherein in item 1) and 2)

R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy,

or a pharmaceutically acceptable salt, stereoisomer, tautomer or solvate of any of these compounds.

The present invention further relates to a new compound as defined above for use as a drug or to a pharmaceutical composition comprising a new compound as defined above.

More particularly, the new compound is for use for the treatment of a disease selected from the group consisting of a cancer, a metabolic disease, especially diabetes mellitus including type 1 and type 2 diabetes mellitus, insulin resistance, hyperglycemia, hyperinsulinemia, glucose intolerance, hypertension, NAFLD, NASH and obesity, polycystic ovary syndrome, metabolic syndrome, cardiovascular diseases including hypertension, atherosclerosis and arteriosclerosis, a secondary mitochondrial disorder due to copper overload including Indian childhood cirrhosis, Wilson's disease and Idiopathic infantile copper toxicosis or due to iron overload including Hereditary Hemochromatosis, Juvenile Hemochromatosis, Neonatal iron storage disease, type I Tyrosinemia and Zellweger syndrome, and mental disorders including schizophrenia, anxiety disorders, mild cognitive disorder, depressive disorder, bipolar disorder, autism spectrum disorder and Fragile X syndrome, an infection by a virus such as a coronavirus or an influenza virus, a neurodegenerative disease or disorder and aging, preferably selected from the group consisting of diabetes mellitus including type 1 and type 2 diabetes mellitus, polycystic ovary syndrome, and glucose intolerance.

Optionally, the compound for use as disclosed above or the new compound as disclosed above is such as R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-.

Optionally, the compound for use as disclosed above or the new compound as disclosed above is such that
R₂, R₃, R₄, R₅, R₆, and R₇ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH); and/or
R₃ and R₆ are H; and/or
R₂ and R₇ are H; and/or
L, and L′ if present, comprises or consists of independently
   —(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 0 to 12, or from 3 to 10, or from 4 to 10, or from 5 to 10, or from 6 to 10; or
   —(CH₂)ₕ—C≡C—C≡C—(CH₂)ᵢ—, with “h” and “i” being an integer independently selected from 0 to 12 and the sum “h” and “i” being an integer selected from 0 to 12, or from 3 to 10, or from 4 to 10, or from 5 to 10, or from 6 to 10; or
   —(CH₂)ₕ—C≡C—(CH₂)ᵢ—, with “h” and “i” being an integer independently selected from 0 to 14 and the sum “h” and “i” being an integer selected from 0 to 14, or from 3 to 12, or from 4 to 10, or from 5 to 10, or from 6 to 10; or
   —(CH₂)ₙ—, with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14; or
   —CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12; or
   —(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 0 to 12, or from 3 to 10, or from 4 to 10, or from 5 to 10, or from 6 to 10.

In a particular aspect, the compound for use as disclosed above or the new compound as disclosed above is such that L, and L′ if present, comprises or consists of independently
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 4 to 12, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 4 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 6 to 14, or from 6 to 10; or
—(CH₂)ₙ—, with “n” being an integer selected from 8 to 16, e.g., 8, 9, 10, 11, 12, 13, 14, 15 or 16, more specifically an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 4 to 12, or from 4 to 10, or from 5 to 10, or from 6 to 10.

In a particular aspect, the compound for use as disclosed above or the new compound as disclosed above is such that L, and L′ if present, comprises or consists of independently
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 7 to 12, or from 7 to 11, or from 8 to 11, or from 9 to 10; or
—(CH₂)ₕ—C≡C—C≡C—(CH₂)ᵢ—, with “h” and “i” being an integer independently selected from 0 to 12 and the sum “h” and “i” being an integer selected from 7 to 12, or from 7 to 11, or from 8 to 11, or from 9 to 10; or
—(CH₂)ₕ—C≡C—(CH₂)ᵢ—, with “h” and “i” being an integer independently selected from 0 to 14 and the sum “h” and “i” being an integer selected from 9 to 14, or from 10 to 13, or from 10 to 12; or
—(CH₂)ₙ—, with “n” being an integer selected from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14; or
   —CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 7 to 12, or from 7 to 11, or from 8 to 11, or from 9 to 10.

In a particular aspect, the compound for use is selected in the group consisting of

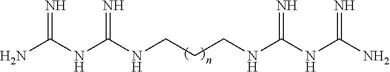

with n being an integer from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

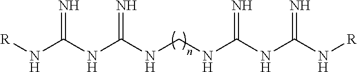

with n being an integer from 8 to 16, e.g., 8, 9, 10, 11, 12, 13, 14, 15 or 16, more specifically an integer selected from 8 to 14, e.g., 8, 9, 10, 11, 12, 13 or 14, or an integer selected from 9 to 14, e.g., 9, 10, 11, 12, 13 or 14, preferably an integer selected from 10 to 14, e.g., 10, 11, 12, 13 or 14, and R being a C₁-C₆alkyl, preferably a C₂-C₆alkyl, e.g., a methyl, ethyl, propyl, butyl, pentyl or hexyl, or a C₀-C₃alkyl-ethynyl (—(CH₂)₀₋₃—C≡CH), preferably —CH₂—C≡CH;

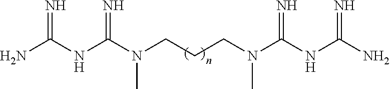

with n being an integer from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

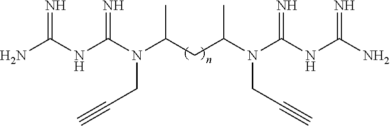

with n being an integer from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

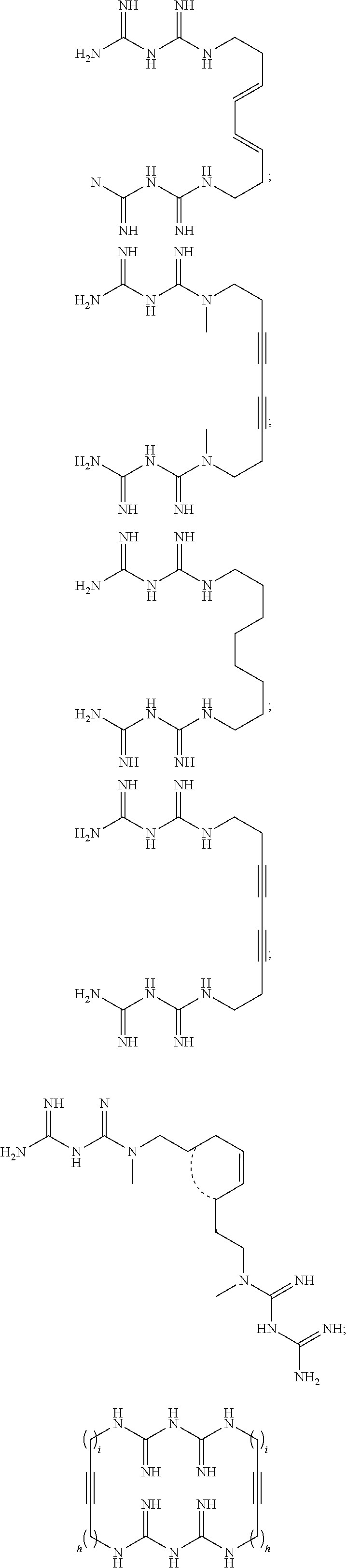

with “h” and “i” being an integer independently selected from 0 to 14 and the sum “h” and “i” being an integer selected from 4 to 14, or from 4 to 12, or from 5 to 10, or from 6 to 10; and

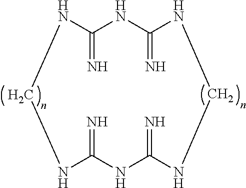

with “n” being an integer selected from 8 to 16, e.g., 8, 9, 10, 11, 12, 13, 14, 15 or 16, more specifically an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

wherein the dotted line being present or absent and being one or two atoms with covalent bonds,

or a pharmaceutically acceptable salt, stereoisomer, tautomer or solvate thereof.

In another particular aspect, the new compound or the new compound for use is selected in the group consisting of

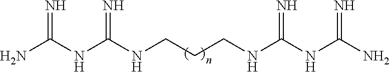

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12;

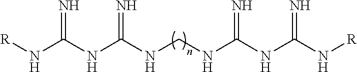

with n being an integer from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14 and R being a C₁-C₆alkyl, preferably a C₂-C₆alkyl, e.g., a methyl, ethyl, propyl, butyl, pentyl or hexyl, or a C₀-C₃alkyl-ethynyl (—(CH₂)₀₋₃—C≡CH), preferably —CH₂—C≡CH;

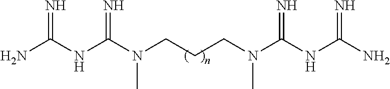

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

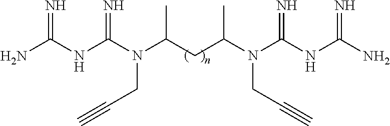

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

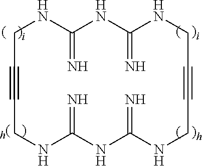

with “h” and “i” being an integer independently selected from 0 to 14 and the sum “h” and “i” being an integer selected from 0 to 14, or from 3 to 13, or from 4 to 12, or from 5 to 10, or from 6 to 10; and

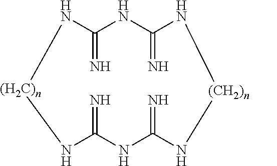

with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

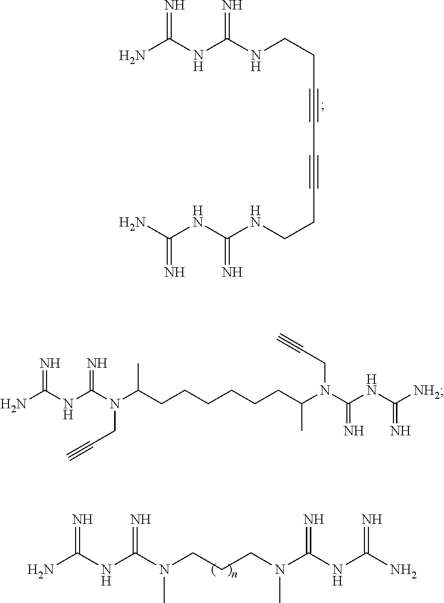

with n being an integer from 2-14 or from 4-14 or from 8-14 or from 8-12 or from 8-10; and

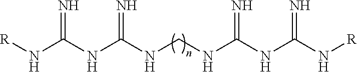

with n being an integer from 6-16, from 8-16 or from 8-12 or from 8-10 and R being an ethyl, a propyl or —CH₂—C≡CH,

or a pharmaceutically acceptable salt, stereoisomer, tautomer or solvate thereof.

### DETAILED DESCRIPTION OF THE INVENTION

### Definitions

If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “C₁-C₃alkyl” more specifically means methyl, ethyl, propyl, or isopropyl. The term “C₁-C₆alkyl” more specifically means methyl, ethyl, propyl, isopropyl, butyl, isobutyl, tert-butyl, pentyl or linear or branched hexyl. In a preferred embodiment, the “alkyl” is a methyl, an ethyl, a propyl, an isopropyl, or a tert-butyl, more preferably a methyl.

The term “alkoxy” or “alkyloxy” corresponds to the alkyl group as above defined bonded to the molecule by an —O— (ether) bond. C₁-C₃alkoxy includes methoxy, ethoxy, propyloxy, and isopropyloxy. C₁-C₆alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “thioalkyl” corresponds to the alkyl group as above defined bounded to the molecule by a —S— (thioether) bound. Thio-C₁-C₆alkyl group includes thio-methyl, thio-ethyl, thio-propyl, thio-butyl, thio-pentyl and thio-hexyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, preferably cyclopropyl. The term “spirocycloalkyl” includes for instance a spirocyclopentyl. In a particular aspect, the term “cycloalkyl” corresponds to a saturated monocycloalkyl group comprising between 3 and 7 atoms of carbons. In a particular aspect, the cycloalkyl group is cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or spiro-connected heterocycloalkyl groups. Representative heterocycloalkyl groups include, but are not limited to 3-dioxolane, benzo [1,3] dioxolyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydro-2H-pyranyl, tetrahydrofuranyl, and tetrahydrothiophenyl. The term “heterocycloalkyl” may also refer to a 5-10 membered bridged heterocyclyl such as 7-oxabicyclo[2,2,1]heptanyl. In a preferred embodiment, the heterocycloalkyl group is a tetrahydro-2H-pyranyl, a tetrahydro-2H-pyranyl, a tetrahydrothiophenyl, a morpholinyl, or a piperazinyl.

The term “aryl” corresponds to a mono- or bi-cyclic aromatic hydrocarbons having from 6 to 12 carbon atoms. For instance, the term “aryl” includes phenyl, biphenyl, or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a preferred embodiment, the heteroaryl group is a thiophenyl, a pyridinyl, a pyrazinyl, or a thiazolyl.

The term “halogen” corresponds to a fluorine, chlorine, bromine, or iodine atom, preferably a fluorine, chlorine or bromine, and more preferably a chlorine or a fluorine.

The expression “substituted by at least” or “substituted by” means that the group is substituted by one or several substituents of the list. For instance, the expression “a C₁-C₆alkyl substituted by at least one halogen” or “a C₁-C₆alkyl substituted by a halogen” may include a fluoromethyl (—CH₂F), a difluoromethyl (—CHF₂), or a trifluoromethyl (—CF₃).

By “—CO—” or “—C(O)—”, it refers to an oxo group. By “—SO—” or “—S(O)—”, it refers to a sulfinyl group. By “—SO₂—” or “—S(O₂)—”, it refers to a sulfonyl group.

The expression “optionally substituted” means that the group is not substituted or substituted by one or several substituents of the list.

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers.

The “tautomers” are isomeric compounds that differ only in the position of the protons and the electrons.

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine). In a preferred embodiment, the salt is selected from the group consisting of sodium and potassium salt.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheeps and non-human primates, among others.

Within the context of the invention, the term treatment denotes curative, symptomatic, and preventive treatment. Pharmaceutical compositions, kits, products and combined preparations of the invention can be used in humans with a disease or disorder. The pharmaceutical compositions, kits, products and combined preparations of the invention will not necessarily cure the patient but will delay or slow the progression or prevent further progression of the disease or disorder, and/or ameliorating thereby the patients' condition. In treating the disease or disorder, the pharmaceutical composition of the invention is administered in a therapeutically effective amount.

Whenever within this whole specification “treatment of a disease or disorder” or the like is mentioned with reference to the pharmaceutical composition of the invention, there is meant: a) a method for treating a disease or disorder, said method comprising administering a pharmaceutical composition of the invention to a subject in need of such treatment; b) the use of a pharmaceutical composition of the invention for the treatment of a disease or disorder; c) the use of a pharmaceutical composition of the invention for the manufacture of a medicament for the treatment of a disease or disorder; and/or d) a pharmaceutical composition of the invention for use in the treatment a disease or disorder.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

By “therapeutically effective amount”, it is meant the quantity of the pharmaceutical composition of the invention which prevents, removes or reduces the deleterious effects of a disease or disorder in mammals, including humans, alone or in combination with the other active ingredients of the pharmaceutical composition, kit, product or combined preparation.

It is understood that the administered dose may be lower for each compound in the composition to the “therapeutic effective amount” define for each compound used alone or in combination with other treatments than the combination described here. The “therapeutic effective amount” of the composition will be adapted by those skilled in the art according to the patient, the pathology, the mode of administration, etc.

As used herein, the term “pharmaceutically acceptable excipient” refers to any ingredient except active ingredients which are present in a pharmaceutical composition. Its addition may be aimed to confer a particular consistency or other physical or gustative properties to the final product. A pharmaceutically acceptable excipient must be devoid of any interaction, in particular chemical, with the active ingredients.

Compounds for Use

The compounds of the present invention have a structure of formula (I), wherein the formula (I) is

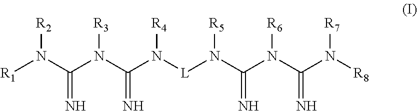

with
R₁, R₂, R₃, R₄, R₅, R₆, R₇ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;
or
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group, and R₁ and R₈ forming together a linker -L′-,
-L- and -L′-, if present, being independently a linear hydrocarbon chain of 4 to 16 carbons optionally interrupted by
   a heteroatom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy,

or a pharmaceutically acceptable salt, stereoisomer, tautomer or solvate thereof.

In a particular aspect, the compounds of the present invention have a structure of formula (I), wherein the formula (I) is

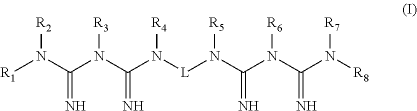

with
R₁, R₂, R₃, R₄, R₅, R₆, R₇ and R₈ being independently selected in the group consisting of H, a C₁-C₆alkyl, a C₀-C₃alkyl-ethynyl (—(CH₂)₀₋₆—C≡CH), a C₃-C₁₀cycloalkyl, and a C₃-C₁₀cycloheteroalkyl, said alkyl, cycloalkyl, or cycloheteroalkyl, being optionally substituted by a R group or a R′ group;
or
R₂, R₃, R₄, R₅, R₆, and R₇ being independently selected in the group consisting of H, a C₁-C₆alkyl, a C₀-C₃alkyl-ethynyl (—(CH₂)₀₋₆—C≡CH), a C₃-C₁₀cycloalkyl, and a C₃-C₁₀cycloheteroalkyl, said alkyl, cycloalkyl, or cycloheteroalkyl, being optionally substituted by a R group or a R′ group, and R₁ and R₈ forming together a linker -L′-,
-L- and -L′-, if present, being independently a linear hydrocarbon chain of 4 to 16 carbons, preferably 8 to 16 carbons, optionally interrupted by
   a heteroatom, preferably an oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl and a heterocycloalkyl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl and a C₃-C₁₀cycloheteroalkyl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy,

or a pharmaceutically acceptable salt, stereoisomer, tautomer or solvate thereof.

In a particular aspect, the compounds of the present invention have a structure of formula (I), wherein the formula (I) is

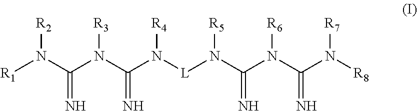

with
R₁, R₂, R₃, R₄, R₅, R₆, R₇ and R₈ being independently selected in the group consisting of H, a C₁-C₆alkyl, a C₀-C₃alkyl-ethynyl (—(CH₂)₀₋₆—C≡CH), said alkyl being optionally substituted by a R group or a R′ group;
or
R₂, R₃, R₄, R₅, R₆, and R₇ being independently selected in the group consisting of H, a C₁-C₆alkyl, a C₀-C₃alkyl-ethynyl (—(CH₂)₀₋₆—C≡CH), said alkyl being optionally substituted by a R group or a R′ group, and R₁ and R₈ forming together a linker -L′-,
-L- and -L′-, if present, being independently a linear hydrocarbon chain of 4 to 16 carbons, preferably 8 to 16 carbons, optionally interrupted by
   a heteroatom, preferably an oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—);
said linear hydrocarbon chain being optionally substituted by a group R or R′;

with R being selected from the group consisting of a C₁-C₆ alkyl optionally substituted by at least one halogen, and a C₁-C₆alkyloxy optionally substituted by at least one halogen, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy, or a pharmaceutically acceptable salt, stereoisomer, tautomer or solvate thereof.

Optionally, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH; and there is no substitution by a group R′.

In one aspect, R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₁ and R₈ are the same or are different.

In one aspect, R₃ and R₆ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. Optionally, R₁ and R₈ are independently selected in the group consisting of H, a C₁-C₆ alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-.

In another aspect, R₂, R₃, R₆, and R₇ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-.

In a specific aspect, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-. Preferably, R₄ and R₅ are —CH₂-ethynyl (—CH₂—C≡CH) or one of R₄ and R₅ is —CH₂-ethynyl (—CH₂—C≡CH) and the other is H.

In another specific aspect, R₂, R₃, R₆, and R₇ are H, R₄ and R₅ are a C₁-C₃alkyl or one of R₄ and R₅ is a C₁-C₃alkyl, preferably a methyl, and the other is H, and either R₁ and R₈ are H or they form together a linker -L′-.

In another specific aspect, R₂, R₃, R₄, R₅, R₆, and R₇ are H and R₁ and R₈ are a C₁-C₆alkyl or a C₀-C₃alkyl-ethynyl (—(CH₂)₀₋₃—C≡CH).

In a very specific aspect, R₂, R₃, R₄, R₅, R₆, and R₇ are H and either R₁ and R₈ are H or they form together a linker -L′-.

L and L′, if present, are a linear hydrocarbon chain of 4 to 16 carbons, preferably 8 to 16 carbons, optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

and being optionally substituted by a group R or R′,

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

In a specific aspect, L and L′, if present, are a linear hydrocarbon chain of 8 to 16 carbons optionally interrupted by
an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

and being optionally substituted by a group R or R′,

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

In one aspect, L and L′, if present, are a linear hydrocarbon chain of4 to 16 carbons, preferably of 8 to 16 carbons, optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl and a heterocycloalkyl, said ring being optionally substituted by a group R or R′;

and being optionally substituted by a group R or R′,

with R being selected from the group consisting of a C₁-C₆alkyl optionally substituted by at least one halogen, a C₁-C₆alkyloxy optionally substituted by at least one halogen, a C₃-C₁₀cycloalkyl and a C₃-C₁₀cycloheteroalkyl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

In another aspect, L and L′, if present, are a linear hydrocarbon chain of 4 to 16 carbons, preferably of 8 to 16 carbons, optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—);

and being optionally substituted by a group R or R′,

with R being selected from the group consisting of a C₁-C₆alkyl optionally substituted by at least one halogen, and a C₁-C₆alkyloxy optionally substituted by at least one halogen, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH; and there is no substitution by a group R′.

Optionally, the hydrocarbon chain may include one or several double or triple bonds.

Optionally, the interrupting heteroatom can be an oxygen (—O—), a sulfur (—S—) or a nitrogen (—NR— with R being H or C₁-C₃ alkyl).

In a preferred aspect, L and L′, if present, are not interrupted by a heteroatom.

L and L′, if present, are such that they allow the proper arrangement of the two biguanidyl radicals so as to form stable complex comprising the two biguanidyl radicals with one copper or iron cation. Optionally, L and L′, if present, are designed so as to increase the lipophilicity of the compound.

Optionally, the linear hydrocarbon chain is of 4 to 16 carbons, e.g., 4, 5, 6, 7, 8, 9, 10, 11, 12, 13, 14, 15 or 16, more specifically of 6 to 15 carbons, e.g., 6, 7, 8, 9, 10, 11, 12, 13, 14 or 15, or of 7 to 14 carbons, e.g., 7, 8, 9, 10, 11, 12, 13 or 14, or of 8 to 14 carbons, e.g., 8, 9, 10, 11, 12, 13, or 14, or of 9 to 14 carbons, e.g., 9, 10, 11, 12, 13, or 14, preferably of 10 to 14 carbons, e.g., 10, 11, 12, 13, or 14.

Optionally, the linear hydrocarbon chain is of 8 to 16 carbons, e.g., 8, 9, 10, 11, 12, 13, 14, 15 or 16, more specifically of 8 to 14 carbons, e.g., 8, 9, 10, 11, 12, 13, or 14, or of 9 to 14 carbons, e.g., 9, 10, 11, 12, 13, or 14, preferably of 10 to 14 carbons, e.g., 10, 11, 12, 13, or 14.

Optionally, the hydrocarbon chain can be interrupted by one or several oxygens and may comprise one or a plurality of (CH₂—CH₂—O—) groups. For instance, the hydrocarbon chain could include 1, 2, 3, 4 and 5 (CH₂—CH₂—O—) groups, especially consecutive groups.

Optionally, L and L′ if present may comprise or consist of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being H, a group R′ or forming together a six-member ring, optionally substituted by one or two R′ groups or fused with a cycloalkyl, an aryl, a cycloheteroalkyl or a heteroaryl having 3 to 6 members, with Rₑ and R_{f} being independently H or a group R′, “f” and “g” being an integer independently selected from 0 to 12 and the sum “f” and “g” being an integer selected from 0 to 12; or
—(CH₂)ₕ—C≡C—C≡C—(CH₂)ᵢ—, with “h” and “i” being an integer independently selected from 0 to 12 and the sum “h” and “i” being an integer selected from 0 to 12; or
—(CH₂)ₕ—C≡C—(CH₂)ᵢ—, with “h” and “i” being an integer independently selected from 0 to 14 and the sum “h” and “i” being an integer selected from 0 to 14; or
—(CH₂)ₙ—, with “n” being an integer selected from 4 to 16; or
—CRₐ—(CH₂)ₙ—CR_{b}—, with Rₐ and R_{b} being H, or a group R′ and with “n” being an integer selected from 2 to 14; or
—(CH₂)ⱼ-cycloalkyl-(CH₂)ₖ— or —(CH₂)ⱼ-heterocycloalkyl-(CH₂)ₖ—, with “j” and “k” being an integer independently selected from 0 to 12 and the sum “j” and “k” being an integer selected from 0 to 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being H, a group R′ or forming together a 4-6 member ring, optionally substituted by one or two R′ groups or fused with a cycloalkyl, an aryl, a cycloheteroalkyl or a heteroaryl having 3 to 6 members, with Rₑ and R_{f} being independently H or a group R′, “p” and “q” being an integer independently selected from 0 to 12 and the sum “p” and “q” being an integer selected from 0 to 12;

with R′ being selected from the group consisting of a halogen, a hydroxyl, a thiol, a cyano, an ethynyl (—C≡CH), a nitro, an amino (—NH2), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₆—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

Optionally, L and L′ if present may comprise or consist of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being H, a group R′ or forming together a six-member ring, optionally substituted by one or two R′ groups or fused with a cycloalkyl, an aryl, a cycloheteroalkyl or a heteroaryl having 3 to 6 members, with Rₑ and R_{f} being independently H or a group R′, “f” and “g” being an integer independently selected from 0 to 12 and the sum “f” and “g” being an integer selected from 4 to 12; or
—(CH₂)ₕ—C≡C—C≡C—(CH₂)ᵢ—, with “h” and “i” being an integer independently selected from 0 to 12 and the sum “h” and “i” being an integer selected from 4 to 12; or
—(CH₂)ₕ—C≡C—(CH₂)ᵢ—, with “h” and “i” being an integer independently selected from 0 to 14 and the sum “h” and “i” being an integer selected from 6 to 14; or
—(CH₂)ₙ—, with “n” being an integer selected from 8 to 16; or
—CRₐ—(CH₂)ₙ—CR_{b}—, with Rₐ and R_{b} being H, or a group R′ and with “n” being an integer selected from 6 to 14; or
—(CH₂)ⱼ-cycloalkyl-(CH₂)ₖ— or —(CH₂)ⱼ-heterocycloalkyl-(CH₂)ₖ—, with “j” and “k” being an integer independently selected from 0 to 12 and the sum “j” and “k” being an integer selected from 8 to 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being H, a group R′ or forming together a 4-6 member ring, optionally substituted by one or two R′ groups or fused with a cycloalkyl, an aryl, a cycloheteroalkyl or a heteroaryl having 3 to 6 members, with Rₑ and R_{f} being independently H or a group R′, “p” and “q” being an integer independently selected from 0 to 12 and the sum “p” and “q” being an integer selected from 4 to 12;

with R′ being selected from the group consisting of a halogen, a hydroxyl, a thiol, a cyano, an ethynyl (—C≡CH), a nitro, an amino (—NH2), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₆—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₆—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆ alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₆—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen.

Optionally, R′ is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH.

In a specific aspect, L, and L′ if present, comprises or consists of independently
—(CH₂)_{f}—CRₐ═CRₑ—CR_{f}═CR_{b}—(CH₂)_{g}—, preferably —(CH₂)_{f}—CRₐ═ independently ═CR_{b}—(CH₂)_{g}—, with Rₐ and R_{b} being H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 0 to 12, or from 3 to 10, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 0 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 0 to 14, or from 3 to 12, or from 4 to 10, or from 5 to 10, or from 6 to 10; or
—(CH₂)ₙ—, with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 0 to 12, or from 3 to 10, or from 4 to 10, or from 5 to 10, or from 6 to 10.

In a specific aspect, L, and L′ if present, comprises or consists of independently
—(CH₂)_{f}—CRₐ═CRₑ—CR_{f}═CR_{b}—(CH₂)_{g}—, preferably —(CH₂)_{f}—CRₐ═ independently ═CR_{b}—(CH₂)_{g}—, with Rₐ and R_{b} being H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 4 to 12, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 4 to 12, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 6 to 14, or from 6 to 10; or
—(CH₂)ₙ—, with “n” being an integer selected from 8 to 16, e.g., 8, 9, 10, 11, 12, 13, 14, 15 or 16, more specifically an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 4 to 12, or from 4 to 10, or from 5 to 10, or from 6 to 10.

In a specific aspect, L, and L′ if present, comprises or consists of independently
—(CH₂)_{f}—CRₐ═CH—CH═CR_{b}—(CH₂)_{g}—, with Rₐ and R_{b} being H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH, “f” and “g” being an integer independently selected from 0 to 12 and the sum “f” and “g” being an integer selected from 7 to 12, or from 7 to 11, or from 8 to 11, or from 9 to 10; or
—(CH₂)ₕ—C≡C—C≡C—(CH₂)ᵢ—, with “h” and “i” being an integer independently selected from 0 to 12 and the sum “h” and “i” being an integer selected from 7 to 12, or from 7 to 11, or from 8 to 11, or from 9 to 10; or
—(CH₂)ₕ—C≡C—(CH₂)ᵢ—, with “h” and “i” being an integer independently selected from 0 to 14 and the sum “h” and “i” being an integer selected from 9 to 14, or from 10 to 13, or from 10 to 12; or
—(CH₂)ₙ—, with “n” being an integer selected from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14; or
—CRₐ—(CH₂)ₙ—CR_{b}—, with Rₐ and R_{b} being H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH, and with “n” being an integer selected from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12; or
—(CH₂)ₚ—CHR_{c}—CH═CH—CHR_{d}—(CH₂)_{q}—, with R_{c} and R_{d} being H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH, “p” and “q” being an integer independently selected from 0 to 12 and the sum “p” and “q” being an integer selected from 7 to 12, or from 7 to 11, or from 8 to 11, or from 9 to 10.

In a specific aspect, L and L′, if present, are —(CH₂)_{f}—CRₐ═CH—CH═CR_{b}—(CH₂)_{g}—, with Rₐ and R_{b} being H, a group R′ or forming together a six-member ring, optionally substituted by one or two R′ groups or fused with a cycloalkyl, an aryl, a cycloheteroalkyl or a heteroaryl having 3 to 6 members, “f” and “g” being an integer independently selected from 0 to 12 and the sum “f” and “g” being an integer selected from 0 to 12. Optionally, Rₐ and R_{b} are H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH. In a specific aspect, Rₐ and R_{b} are H. More specifically, the sum “f” and “g” can be an integer selected from 3 to 10, or from 4 to 10, or from 5 to 10, or from 6 to 10. “f” and “g” can be a different integer or can be the same integer. Optionally, the sum “f” and “g” can be an integer selected from the group consisting of 3, 4, 5, 6, 7, 8, 9 or 10. Optionally, the integers “f” and “g” in -L- and -L′- can be different or the same. More specifically, R₂, R₃, R₆, and R₇ can be H, R₄ and R₅ can be independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH), and R₁ and R₈ are independently selected in the group consisting of H, a C₁-C₆ alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₂, R₃, R₄, R₅, R₆, and R₇ are H and either R₁ and R₈ are H or they form together a linker -L′-. Optionally, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-.

In another specific aspect, L and L′, if present, are —(CH₂)ₕ—C≡C—C≡C—(CH₂)ᵢ—, with “h” and “i” being an integer independently selected from 0 to 12 and the sum “h” and “i” being an integer selected from 0 to 12. More specifically, the sum “h” and “i” can be an integer selected from 3 to 10, or from 4 to 10, or from 5 to 10, or from 6 to 10. “h” and “i” can be a different integer or can be the same integer. Optionally, the sum “h” and “i” can be an integer selected from the group consisting of 3, 4, 5, 6, 7, 8, 9 or 10. Optionally, the integers “h” and “i” in -L- and -L′- can be different or the same. In this specific aspect, R₁, R₂, R₃, R₄, R₅, R₆, R₇ and R₈ can be as defined above in any aspect. More specifically, R₂, R₃, R₆, and R₇ can be H, R₄ and R₅ can be independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH), and R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₂, R₃, R₄, R₅, R₆, and R₇ are H and either R₁ and R₈ are H or they form together a linker -L′-. Optionally, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-.

In another specific aspect, L and L′, if present, are —CRₐ—(CH₂)ₙ—CR_{b}—, with Rₐ and R_{b} being H, or a group R′ and with “n” being an integer selected from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12. Optionally, the integers n of L and L′ are the same. Optionally, the integers n of L and L′ differ of 1, 2, 3 or 4 (i.e., a first n is 10 and the other is 8 or 12 if it differs of 2, 9 or 11 if it differs of 1). Optionally, Rₐ and R_{b} are a C₁-C₃alkyl, e.g., a methyl, ethyl or propyl. In this specific aspect, R₁, R₂, R₃, R₄, R₅, R₆, R₇ and R₈ can be as defined above in any aspect. More specifically, R₂, R₃, R₆, and R₇ can be H, R₄ and R₅ can be independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH), and R₁ and R₈ are independently selected in the group consisting of H, a C₁-C₆ alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₂, R₃, R₄, R₅, R₆, and R₇ are H and either R₁ and R₈ are H or they form together a linker -L′-. Optionally, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-.

In an additional specific aspect, L and L′, if present, are —(CH₂)ₕ—C≡C—(CH₂)ᵢ—, with “h” and “i” being an integer independently selected from 0 to 14 and the sum “h” and “i” being an integer selected from 0 to 14. More specifically, the sum “h” and “i” can be an integer selected from 3 to 13, or from 4 to 12, or from 5 to 10, or from 6 to 10. In a very specific aspect, “h” and “i” are 3 or 4. “h” and “i” can be a different integer or can be the same integer. Optionally, the sum “h” and “i” can be an integer selected from the group consisting of 3, 4, 5, 6, 7, 8, 9 or 10. Optionally, the integers “h” and “i” in -L- and -L′- can be different or the same. In this specific aspect, R₁, R₂, R₃, R₄, R₅, R₆, R₇ and R₈ can be as defined above in any aspect. More specifically, R₂, R₃, R₆, and R₇ can be H, R₄ and R₅ can be independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH), and R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₂, R₃, R₄, R₅, R₆, and R₇ are H and either R₁ and R₈ are H or they form together a linker -L′-. Optionally, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-.

In a very specific aspect, L and L′, if present, are —(CH₂)ₙ— with n being independently an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14. Optionally, the integers n of L and L′ are the same. Optionally, the integers n of L and L′ differ of 1, 2, 3 or 4 (i.e., a first n is 10 and the other is 8 or 12 if it differs of 2, 9 or 11 if it differs of 1).

In this very specific aspect, R₁ and R₈ form together a linker -L′- and the -L- and -L′- are —(CH₂)ₙ— with n being independently an integer selected from 4 to 16, e.g., 4, 5, 6, 7, 8, 9, 10, 11, 12, 13, 14, 15 or 16, or from 5 to 14, e.g., 5, 6, 7, 8, 9, 10, 11, 12, 13 or 14, or from 6 to 12, e.g., 6, 7, 8, 9, 10, 11 or 12. In this very specific aspect, R₁, R₂, R₃, R₄, R₅, R₆, R₇ and R₈ can be as defined above in any aspect. More specifically, R₂, R₃, R₆, and R₇ can be H, R₄ and R₅ can be independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH), and R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₂, R₃, R₄, R₅, R₆, and R₇ are H and either R₁ and R₈ are H or they form together a linker -L′-. Optionally, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-.

Optionally, the compound can be selected in the group consisting of

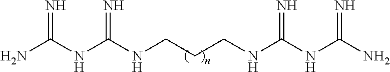

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

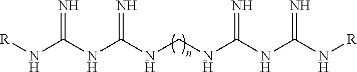

with n being an integer from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13 or 14, or an integer selected from 9 to 14, e.g., 9, 10, 11, 12, 13 or 14, preferably an integer selected from 10 to 14, e.g., 10, 11, 12, 13 or 14; preferably, R is selected from the group consisting of an ethyl, a propyl and —CH2-C≡CH;

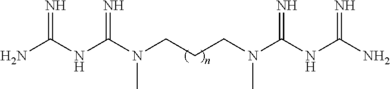

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

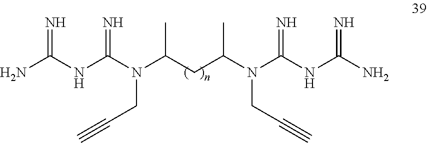

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

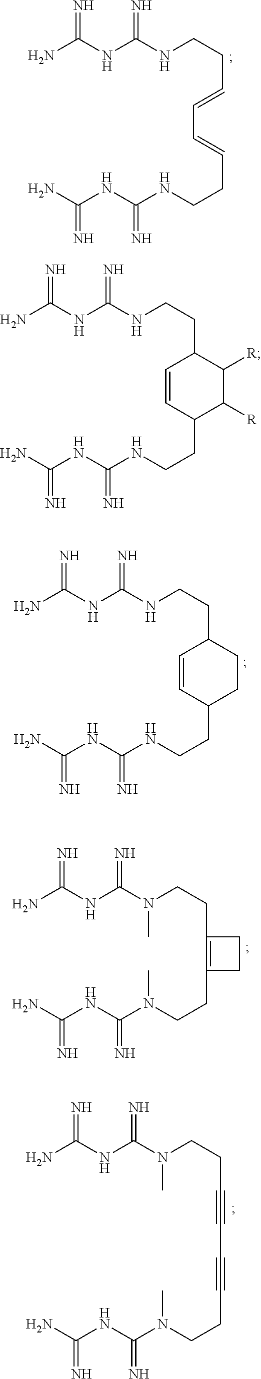

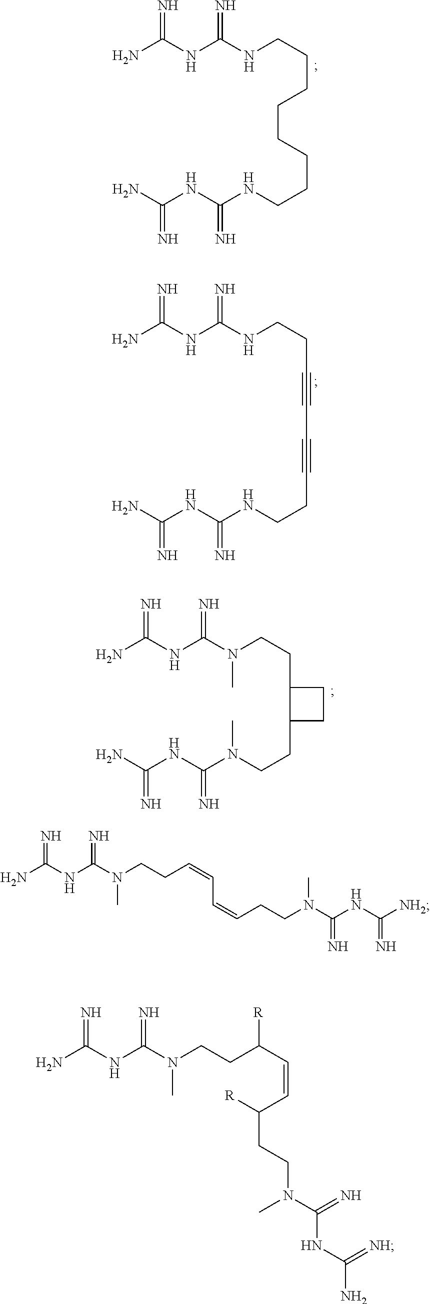

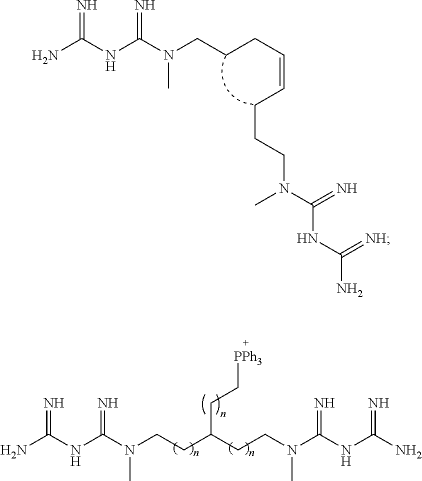

with n being independently an integer selected from 1 to 13, preferably from 3 to 6;

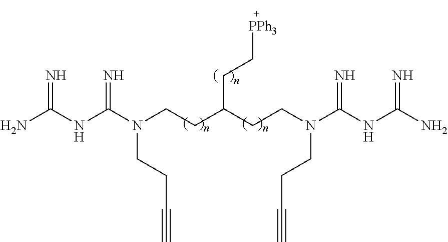

with n being independently an integer selected from 1 to 13, preferably from 3 to 6;

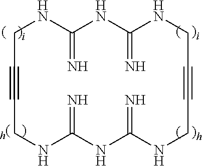

with “h” and “i” being an integer independently selected from 0 to 14 and the sum “h” and “i” being an integer selected from 0 to 14, or from 3 to 13, or from 4 to 12, or from 5 to 10, or from 6 to 10; and

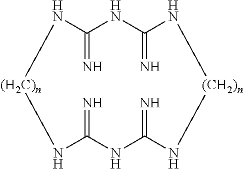

with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

wherein
the dotted line being present or absent and being one or two atoms with covalent bonds; and
R is selected from the group consisting of a halogen, a hydroxyl, a thiol, a cyano, a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, a C₀-C₃alkyl-ethynyl (—(CH₂)₀₋₃—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy;
or a pharmaceutically acceptable salt, stereoisomer, tautomer or solvate thereof.

Optionally, R is selected from the group consisting of a halogen, a hydroxyl, a thiol, a cyano, a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₃—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R is selected from the group consisting of a halogen, a hydroxyl, a thiol, a cyano, a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-ethynyl (—(CH₂)₀₋₃—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆ alkyl optionally substituted by at least one halogen and said cycloalkyl or cycloheteroalkyl being optionally substituted by a halogen, a hydroxyl, a cyano, a nitro, an amino, or a C₁-C₃alkoxy.

In a particular aspect, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH.

In a particular aspect, the compound can be selected in the group consisting of

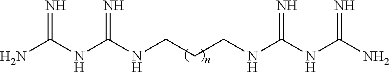

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

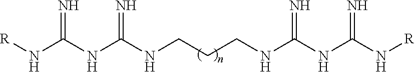

with n being an integer from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13 or 14, or an integer selected from 9 to 14, e.g., 9, 10, 11, 12, 13 or 14, preferably an integer selected from 10 to 14, e.g., 10, 11, 12, 13 or 14, and R being a C₁-C₆alkyl, preferably a C₂-C₆alkyl, e.g., a methyl, ethyl, propyl, butyl, pentyl or hexyl, or a C₀-C₃alkyl-ethynyl (—(CH₂)₀₋₃—C≡CH), preferably —CH₂—C≡CH;

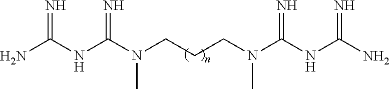

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

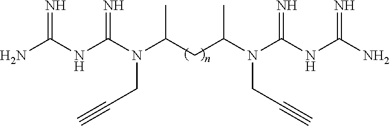

with n being an integer from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

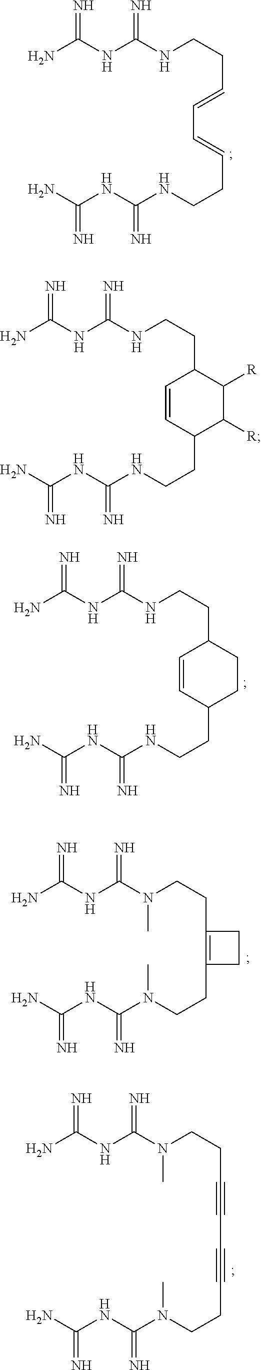

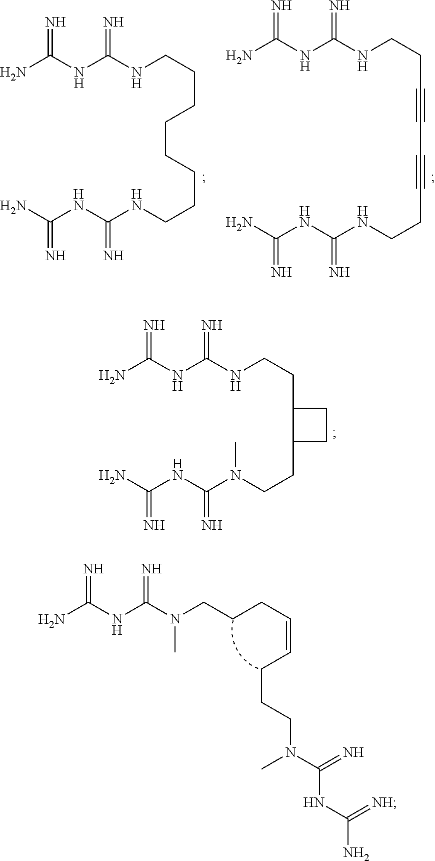

with “h” and “i” being an integer independently selected from 0 to 14 and the sum “h” and “i” being an integer selected from 0 to 14, or from 3 to 13, or from 4 to 12, or from 5 to 10, or from 6 to 10; and

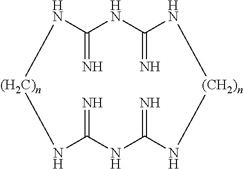

with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

wherein the dotted line being present or absent and being one or two atoms with covalent bonds,

or a pharmaceutically acceptable salt, stereoisomer, tautomer or solvate thereof.

In a specific aspect, the compound can be selected in the group consisting of

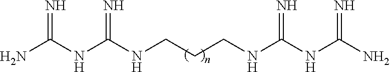

with n being an integer from 6 to 14, e.g., 6, 7, 8, 9, 10, 11, 12, 13 or 14, more specifically selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

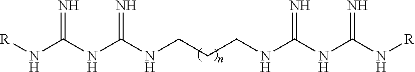

with n being an integer from 8 to 16, e.g., 8, 9, 10, 11, 12, 13, 14, 15 or 16, more specifically an integer selected from 8 to 14, e.g., 8, 9, 10, 11, 12, 13 or 14, or an integer selected from 9 to 14, e.g., 9, 10, 11, 12, 13 or 14, preferably an integer selected from 10 to 14, e.g., 10, 11, 12, 13 or 14, and R being a C₁-C₆alkyl, preferably a C₂-C₆alkyl, e.g., a methyl, ethyl, propyl, butyl, pentyl or hexyl, or a C₀-C₃alkyl-ethynyl (—(CH₂)₀₋₃—C≡CH), preferably —CH₂—C≡CH; preferably, R is selected from the group consisting of an ethyl, a propyl and —CH₂—C≡CH;

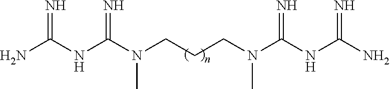

with n being an integer from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

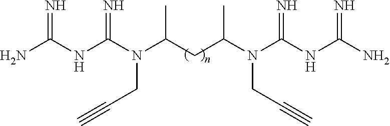

with n being an integer from 6 to 14, e.g., 6, 7, 8, 9, 10, 11, 12, 13 or 14, more specifically an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12;

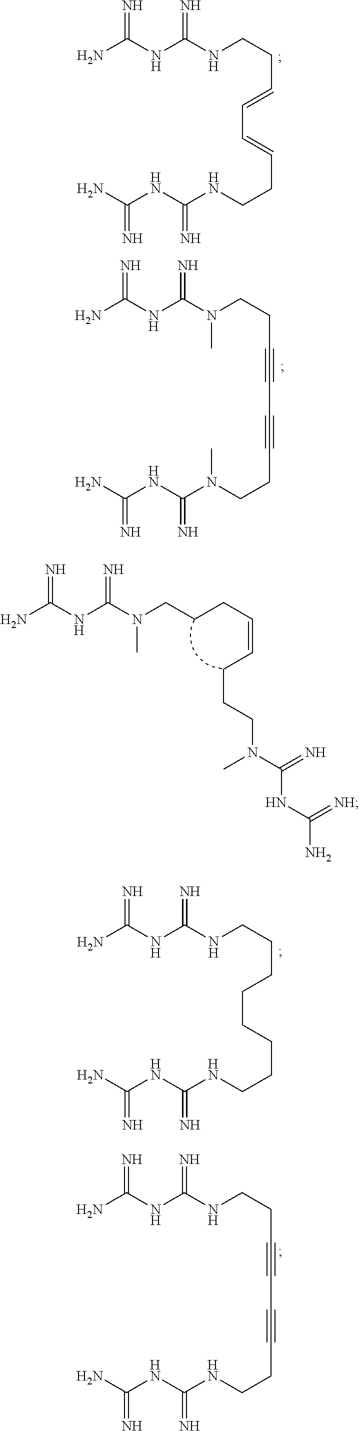

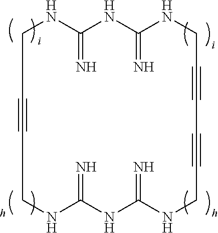

with “h” and “i” being an integer independently selected from 0 to 14 and the sum “h” and “i” being an integer selected from 4 to 14, or from 4 to 12, or from 5 to 10, or from 6 to 10; and

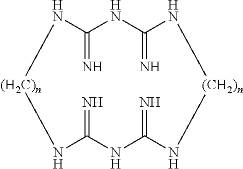

with “n” being an integer selected from 8 to 16, e.g., 8, 9, 10, 11, 12, 13, 14, 15 or 16, more specifically an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

wherein the dotted line being present or absent and being one or two atoms with covalent bonds,

or a pharmaceutically acceptable salt, stereoisomer, tautomer or solvate thereof.

Optionally, in any of the specific aspect disclosed above, R, R₁, R₂, R₇ and R₈ are not aryl.

Optionally, in any of the specific aspect disclosed above, R, R₁, R₂, R₇ and R₈ are not an alkyl substituted by an aryl. Optionally, in any of the specific aspect disclosed above, R, R₁, R₂, R₇ and R₈ are not aryl or an alkyl substituted by an aryl.

Optionally, in any of the specific aspect disclosed above, L is not interrupted by an nitrogen.

Optionally, in any of the specific aspect disclosed above, n is an integer of at least 8, 9, 10, 11 or 12.

Optionally, in any of the specific aspect disclosed above, R₄ and R₅ are H.

Optionally, in any of the specific aspect disclosed above, R₁, R₂, R₇, and R₈ are H.

Optionally, in any of the specific aspect disclosed above, R, R₁, R₂, R₇ and R₈ are not aryl or an alkyl substituted by an aryl and L is not interrupted by an nitrogen. Optionally, R₁, R₂, R₇ and R₈ are H and L is not interrupted by an nitrogen.

Optionally, in any of the specific aspect disclosed above, R, R₁, R₂, R₇ and R₈ are not aryl or an alkyl substituted by an aryl and n is an integer of at least 8, 9, 10, 11 or 12. Optionally, R₁, R₂, R₇ and R₈ are H and n is an integer of at least 8, 9, 10, 11 or 12.

Optionally, in any of the specific aspect disclosed above, R, R₁, R₂, R₇ and R₈ are not aryl or an alkyl substituted by an aryl and R₄ and R₅ are H. Optionally, R₁, R₂, R₇ and R₈ are H and R₄ and R₅ are H.

Optionally, in any of the specific aspect disclosed above, R, R₁, R₂, R₇ and R₈ are not aryl or an alkyl substituted by an aryl, L is not interrupted by an nitrogen and n is an integer of at least 8, 9, 10, 11 or 12. Optionally, R₁, R₂, R₇ and R₈ are H, L is not interrupted by an nitrogen and n is an integer of at least 8, 9, 10, 11 or 12.

Optionally, in any of the specific aspect disclosed above, R, R₁, R₂, R₇ and R₈ are not aryl or an alkyl substituted by an aryl, L is not interrupted by an nitrogen, n is an integer of at least 8, 9, 10, 11 or 12 and R₄ and R₅ are H. Optionally, R₁, R₂, R₇ and R₈ are H, L is not interrupted by an nitrogen, n is an integer of at least 8, 9, 10, 11 or 12 and R₄ and R₅ are H.

In a preferred aspect, the compound can be selected in the group consisting of:

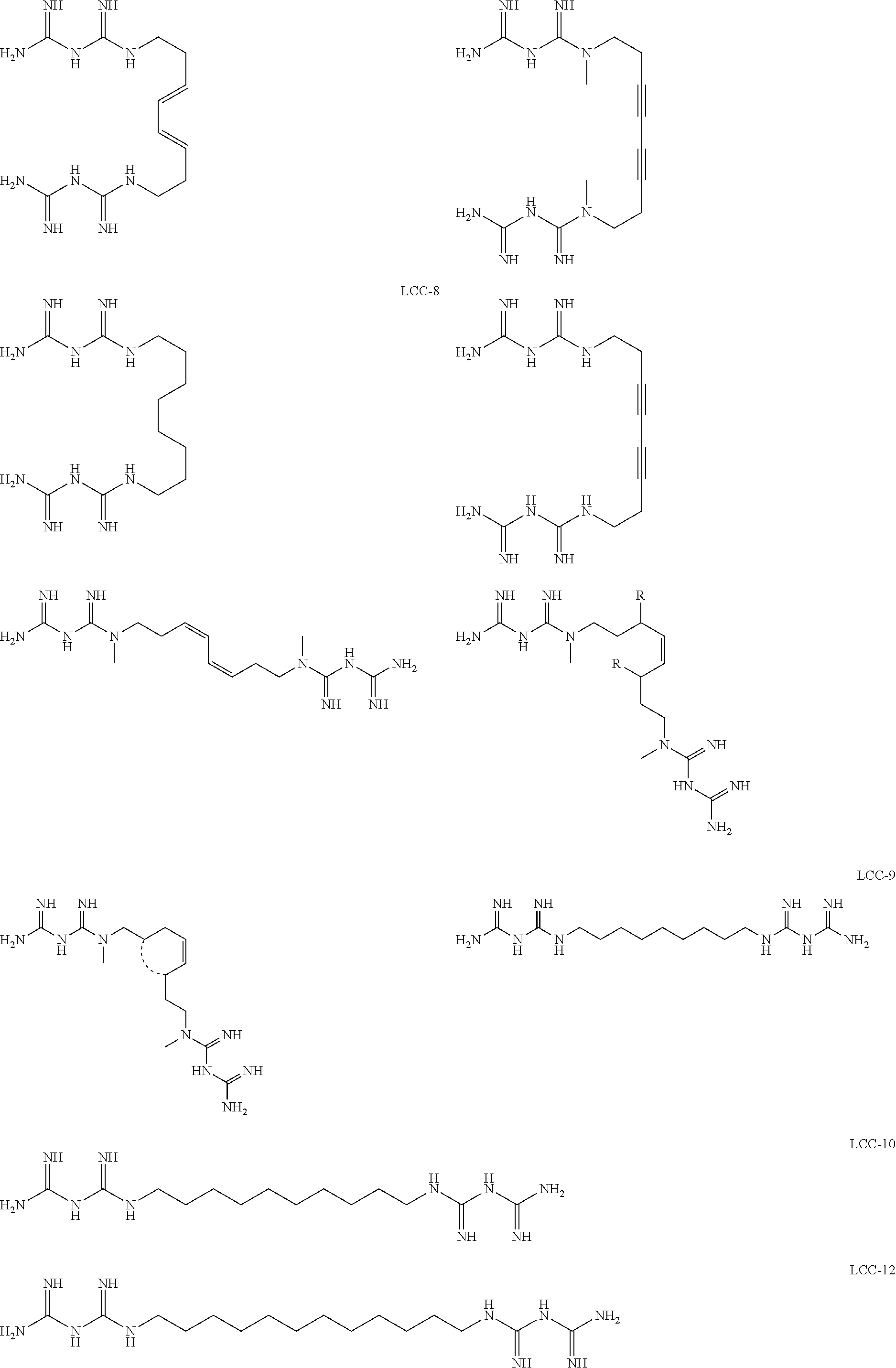

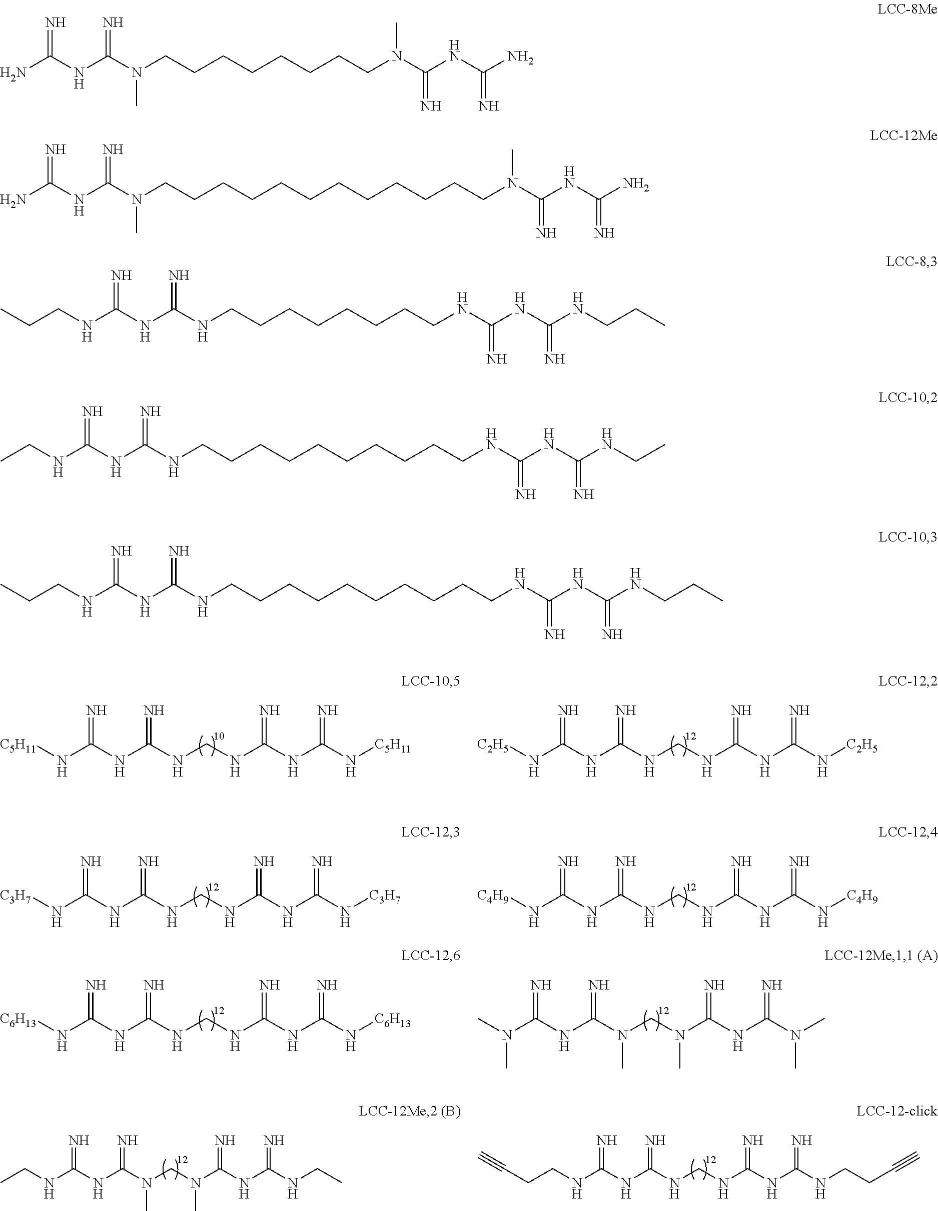

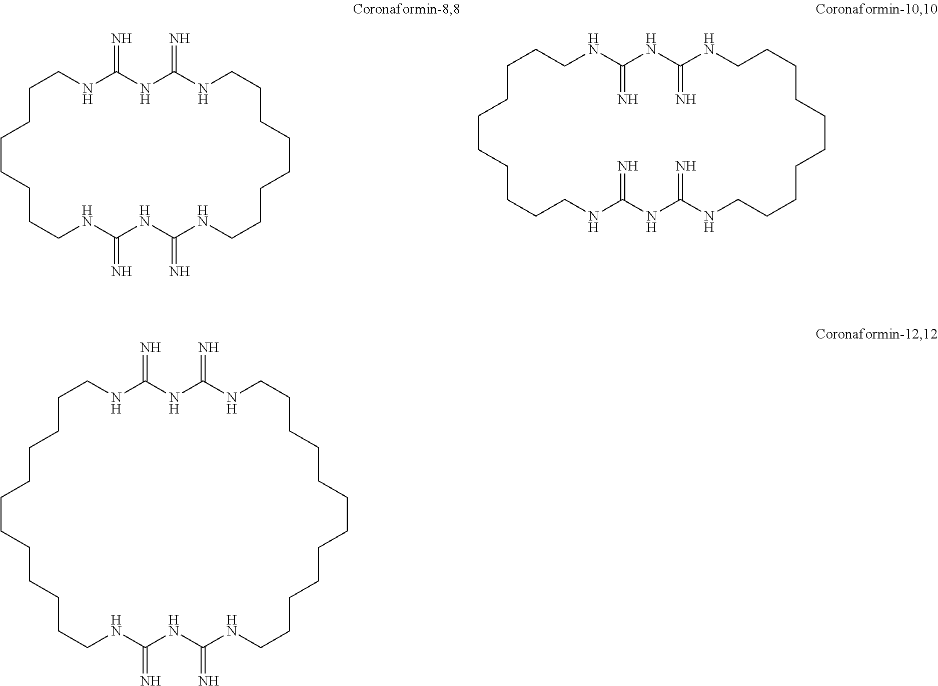 [SMILES: N=C1NCCCCCCCCNC(=N)NC(=N)NCCCCCCCCNC(=N)N1 ; N=C1NCCCCCCCCCCNC(=N)NC(=N)NCCCCCCCCCCNC(=N)N1 ; N=C1NCCCCCCCCCCCCNC(=N)NC(=N)NCCCCCCCCCCCCNC(=N)N1]

or a pharmaceutically acceptable salt, stereoisomer, tautomer or solvate thereof. Preferably, the compound is selected from the group consisting of LCC-8, LCC-9, LCC-10, LCC-12, LCC-8Me, and LCC-12Me, or a pharmaceutically acceptable salt, stereoisomer, tautomer or solvate thereof. More preferably, the compound is selected from the group consisting of LCC-10, LCC-12 and LCC-12Me, or a pharmaceutically acceptable salt, stereoisomer, tautomer or solvate thereof.

In a specific aspect, the compound is in the form of a pharmaceutically acceptable salt, in particular a di-formic acid salt or a di-hydrochloride salt.

New Compounds

The present invention relates to a new compound having a structure of formula (I), wherein the formula (I) is

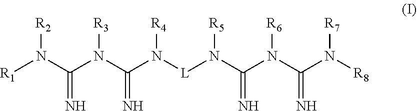

wherein
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;

and

1) R₁ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group; and

-L- being independently a linear hydrocarbon chain of 11 to 16 carbons optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

said linear hydrocarbon chain being optionally substituted by a group R or R′;

or

2) R₁ and R₈ forming together a linker -L′-; and

-L- and -L′- being independently a linear hydrocarbon chain of 4 to 16 carbons optionally interrupted by
a heteroatom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;

said linear hydrocarbon chain being optionally substituted by a group R or R′;

or

3) the compound is selected in the group consisting of

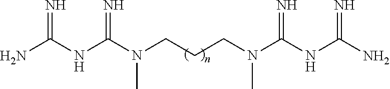

with n being an integer from 2-14 or from 4-14 or from 8-14 or from 8-12 or from 8-10; and

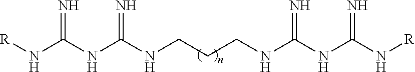

with n being an integer from 6-16, from 8-16 or from 8-12 or from 8-10 and R being an ethyl, a propyl or —CH₂—C≡CH,

wherein in item 1) and 2)

R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃ alkoxy,

or a pharmaceutically acceptable salt, stereoisomer, tautomer or solvate of any of these compounds.

Optionally, the new compound has a structure of formula (I), wherein the formula (I) is

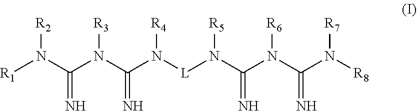

wherein
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, and a C₃-C₁₀cycloheteroalkyl, said alkyl, cycloalkyl, or cycloheteroalkyl, being optionally substituted by a R group or a R′ group;

and

1) R₁ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, and a C₃-C₁₀cycloheteroalkyl, said alkyl, cycloalkyl, or cycloheteroalkyl, being optionally substituted by a R group or a R′ group; and -L- being independently a linear hydrocarbon chain of 11 to 16 carbons optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl and a heterocycloalkyl, said ring being optionally substituted by a group R or R′;

said linear hydrocarbon chain being optionally substituted by a group R or R′;

or

2) R₁ and R₈ forming together a linker -L′-; and

-L- and -L′- being independently a linear hydrocarbon chain of 4 to 16 carbons optionally interrupted by
a heteroatom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl and a heterocycloalkyl, said ring being optionally substituted by a group R or R′;

said linear hydrocarbon chain being optionally substituted by a group R or R′;

or

3) the compound is selected in the group consisting of

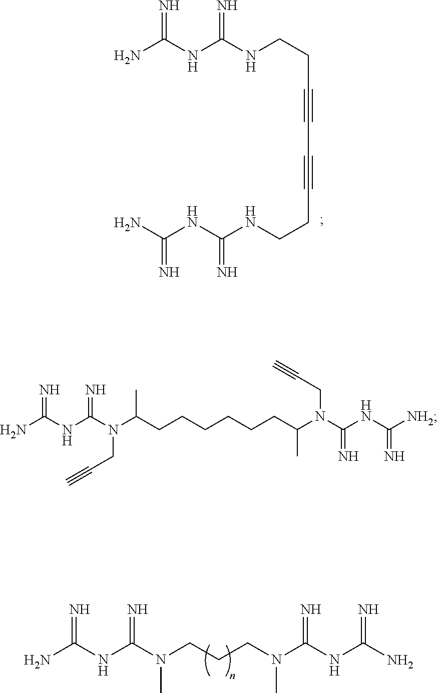

with n being an integer from 2-14 or from 4-14 or from 8-14 or from 8-12 or from 8-10; and

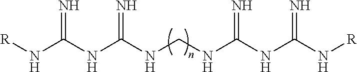

with n being an integer from 6-16, from 8-16 or from 8-12 or from 8-10 and R being an ethyl, a propyl or —CH₂—C≡CH,

wherein in item 1) and 2)

R being selected from the group consisting of a C₁-C₆alkyl optionally substituted by at least one halogen, a C₁-C₆alkyloxy optionally substituted by at least one halogen, a C₃-C₁₀cycloalkyl and a C₃-C₁₀cycloheteroalkyl, the group being optionally substituted by a group R′, and R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy,

or a pharmaceutically acceptable salt, stereoisomer, tautomer or solvate thereof.

Optionally, the new compound has a structure of formula (I), wherein the formula (I) is

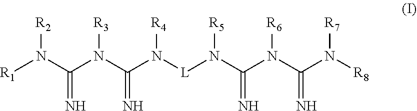

wherein
R₂, R₃, R₄, R₅, R₆, and R₇ being independently selected in the group consisting of H, a C₁-C₆alkyl, a C₀-C₃alkyl-ethynyl (—(CH₂)₀₋₆—C≡CH), said alkyl being optionally substituted by a R group or a R′ group;

and

1) R₁ and R₈ being independently selected in the group consisting of H, a C₁-C₆alkyl, a C₀-C₃alkyl-ethynyl (—(CH₂)₀₋₆—C≡CH), said alkyl being optionally substituted by a R group or a R′ group; and

-L- being independently a linear hydrocarbon chain of 11 to 16 carbons optionally interrupted by
a heteroatom, preferably an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—);

said linear hydrocarbon chain being optionally substituted by a group R or R′;

or

2) R₁ and R₈ forming together a linker -L′-; and

-L- and -L′- being independently a linear hydrocarbon chain of 4 to 16 carbons optionally interrupted by
a heteroatom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or

said linear hydrocarbon chain being optionally substituted by a group R or R′;

or

3) the compound is selected in the group consisting of

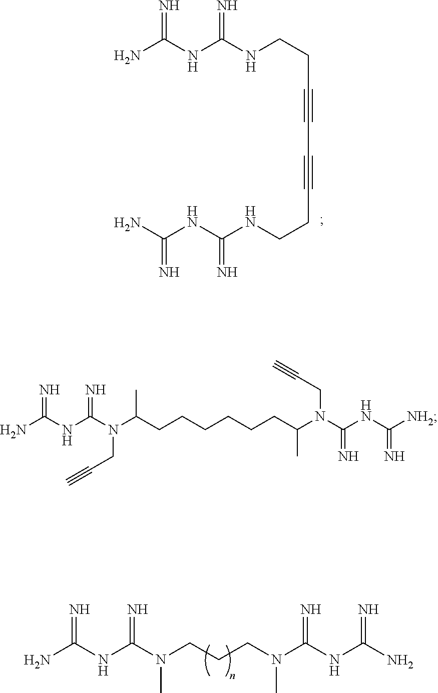

with n being an integer from 2-14 or from 4-14 or from 8-14 or from 8-12 or from 8-10; and

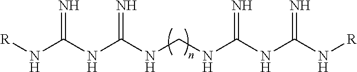

with n being an integer from 6-16, from 8-16 or from 8-12 or from 8-10 and R being an ethyl, a propyl or —CH2-C≡CH,

wherein in item 1) and 2)

R being selected from the group consisting of a C₁-C₆alkyl optionally substituted by at least one halogen, and a C₁-C₆alkyloxy optionally substituted by at least one halogen, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆ alkyl optionally substituted by at least one halogen and said cycloalkyl or cycloheteroalkyl being optionally substituted by a halogen, a hydroxyl, a cyano, a nitro, an amino, or a C₁-C₃alkoxy, or a pharmaceutically acceptable salt, stereoisomer, tautomer or solvate thereof.

Optionally, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH; and there is no substitution by a group R′.

In one aspect, R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-. Optionally, R₁ and R₈ are the same or are different.

In one aspect, R₃ and R₆ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. Optionally, R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-.

In another aspect, R₂, R₃, R₆, and R₇ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH), or they form together a linker -L′-.

In a specific aspect, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and either R₁ and R₈ are H or they form together a linker -L′-. Preferably, R₄ and R₅ are —CH₂-ethynyl (—CH₂—C≡CH) or one of R₄ and R₅ is —CH₂-ethynyl (—CH₂—C≡CH) and the other is H.

In another specific aspect, R₂, R₃, R₆, and R₇ are H, R₄ and R₅ are a C₁-C₃alkyl or one of R₄ and R₅ is a C₁-C₃alkyl, preferably a methyl, and the other is H, and either R₁ and R₈ are H or they form together a linker -L′-.

In another specific aspect, R₂, R₃, R₄, R₅, R₆, and R₇ are H and R₁ and R₈ are a C₁-C₆alkyl or a C₀-C₃alkyl-ethynyl (—(CH₂)₀₋₃—C≡CH).

In a very specific aspect, R₂, R₃, R₄, R₅, R₆, and R₇ are H and either R₁ and R₈ are H or they form together a linker -L′-.

In a more particular aspect, the new compounds of the present invention have a structure of formula (I), wherein the formula (I) is

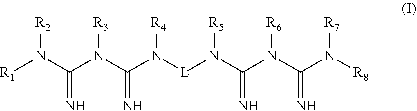

with
R₁, R₂, R₃, R₄, R₅, R₆, R₇ and R₈ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;
-L- being independently a linear hydrocarbon chain of 11 to 16 carbons optionally interrupted by
   a heteroatom, preferably an oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃ alkoxy,

or a pharmaceutically acceptable salt, stereoisomer, tautomer or solvate thereof.

Optionally, the new compounds of the present invention have a structure of formula (I), with
R₁, R₂, R₃, R₄, R₅, R₆, R₇ and R₈ being independently selected in the group consisting of H, a C₁-C₆alkyl, a C₀-C₃alkyl-ethynyl (—(CH₂)₀₋₆—C≡CH), a C₃-C₁₀cycloalkyl, and a C₃-C₁₀cycloheteroalkyl, said alkyl, cycloalkyl, or cycloheteroalkyl, being optionally substituted by a R group or a R′ group;
-L- being independently a linear hydrocarbon chain of 11 to 16 carbons optionally interrupted by
   a heteroatom, preferably an oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl and a heterocycloalkyl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl and a C₃-C₁₀cycloheteroalkyl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy,

or a pharmaceutically acceptable salt, stereoisomer, tautomer or solvate thereof.

Optionally, the new compounds of the present invention have a structure of formula (I), with
R₁, R₂, R₃, R₄, R₅, R₆, R₇ and R₈ being independently selected in the group consisting of H, a C₁-C₆alkyl, a C₀-C₃alkyl-ethynyl (—(CH₂)₀₋₆—C≡CH), said alkyl being optionally substituted by a R group or a R′ group;
-L- being independently a linear hydrocarbon chain of 11 to 16 carbons optionally interrupted by
   a heteroatom, preferably an oxygen atom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—);
said linear hydrocarbon chain being optionally substituted by a group R or R′;

with R being selected from the group consisting of a C₁-C₆alkyl optionally substituted by at least one halogen, and a C₁-C₆alkyloxy optionally substituted by at least one halogen, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy, or a pharmaceutically acceptable salt, stereoisomer, tautomer or solvate thereof.

Optionally, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH; and there is no substitution by a group R′.

In one aspect, R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH). Optionally, R₁ and R₈ are the same or are different.

In one aspect, R₃ and R₆ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. Optionally, R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH).

In another aspect, R₂, R₃, R₆, and R₇ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different. R₁ and R₈ are independently selected in the group consisting of H, a C₁-C₆alkyl and a C₀-C₃alkyl-ethynyl (—(CH₂)₀₋₃—C≡CH), preferably from the group consisting of H, a methyl, an ethyl, a propyl and —CH₂-ethynyl (—CH₂—C≡CH).

In a specific aspect, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H, and R₁ and R₈ are H. Preferably, R₄ and R₅ are —CH₂-ethynyl (—CH₂—C≡CH) or one of R₄ and R₅ is —CH₂-ethynyl (—CH₂—C≡CH) and the other is H.

In another specific aspect, R₂, R₃, R₆, and R₇ are H, R₄ and R₅ are a C₁-C₃alkyl or one of R₄ and R₅ is a C₁-C₃alkyl, preferably a methyl, and the other is H, and R₁ and R₈ are H.

In another specific aspect, R₂, R₃, R₄, R₅, R₆, and R₇ are H and R₁ and R₈ are a C₁-C₆alkyl or a C₀-C₃alkyl-ethynyl (—(CH₂)₀₋₃—C≡CH).

In a very specific aspect, R₁, R₂, R₃, R₄, R₅, R₆, R₇ and R₈ are H.

Optionally, L may comprise or consist of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being H, a group R′ or forming together a six-member ring, optionally substituted by one or two R′ groups or fused with a cycloalkyl, an aryl, a cycloheteroalkyl or a heteroaryl having 3 to 6 members, with Rₑ and R_{f} being independently H or a group R′, “f” and “g” being an integer independently selected from 0 to 12 and the sum “f” and “g” being an integer selected from 0 to 12; or
—(CH₂)ₕ—C≡C—C≡C—(CH₂)ᵢ—, with “h” and “i” being an integer independently selected from 0 to 12 and the sum “h” and “i” being an integer selected from 0 to 12; or
—(CH₂)ₕ—C≡C—(CH₂)ᵢ—, with “h” and “i” being an integer independently selected from 0 to 14 and the sum “h” and “i” being an integer selected from 0 to 14; or
—(CH₂)ₙ—, with “n” being an integer selected from 4 to 16; or
—CRₐ—(CH₂)ₙ—CR_{b}—, with Rₐ and R_{b} being H, or a group R′ and with “n” being an integer selected from 2 to 14; or
—(CH₂)ⱼ-cycloalkyl-(CH₂)ₖ— or —(CH₂)ⱼ-heterocycloalkyl-(CH₂)ₖ—, with “j” and “k” being an integer independently selected from 0 to 12 and the sum “j” and “k” being an integer selected from 0 to 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being H, a group R′ or forming together a 4-6 member ring, optionally substituted by one or two R′ groups or fused with a cycloalkyl, an aryl, a cycloheteroalkyl or a heteroaryl having 3 to 6 members, with Rₑ and R_{f} being independently H or a group R′, “p” and “q” being an integer independently selected from 0 to 12 and the sum “p” and “q” being an integer selected from 0 to 12;

with R′ being selected from the group consisting of a halogen, a hydroxyl, a thiol, a cyano, an ethynyl (—C≡CH), a nitro, an amino (—NH2), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀ cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen.

In a specific aspect, L comprises or consists of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 7 to 12, or from 7 to 11, or from 8 to 11, or from 9 to 10; or
—(CH₂)ₕ—C≡C—C≡C—(CH₂)ᵢ—, with “h” and “i” being an integer independently selected from 0 to 12 and the sum “h” and “i” being an integer selected from 7 to 12, or from 7 to 11, or from 8 to 11, or from 9 to 10; or
—(CH₂)ₕ—C≡C—(CH₂)ᵢ—, with “h” and “i” being an integer independently selected from 0 to 14 and the sum “h” and “i” being an integer selected from 9 to 14, or from 10 to 13, or from 10 to 12; or
—(CH₂)ₙ—, with “n” being an integer selected from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 7 to 12, or from 7 to 11, or from 8 to 11, or from 9 to 10.

Optionally, the compound can be selected in the group consisting of

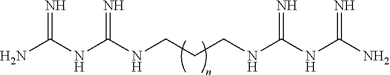

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12;

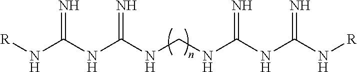

with n being an integer from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14;

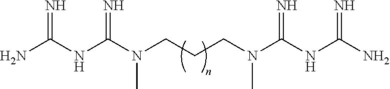

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

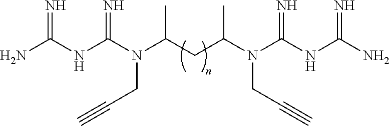

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

wherein R is selected from the group consisting of a halogen, a hydroxyl, a thiol, a cyano, a C₀-C₃alkyl-ethynyl (—(CH₂)₀₋₃—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, a C₀-C₃alkyl-ethynyl (—(CH₂)₀₋₃—C≡CH), C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy;

or a pharmaceutically acceptable salt, stereoisomer, tautomer or solvate thereof.

Optionally, R is selected from the group consisting of a halogen, a hydroxyl, a thiol, a cyano, a C₀-C₃alkyl-ethynyl (—(CH₂)₀₋₃—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀ cycloalkyl, and a C₃-C₁₀ cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R is selected from the group consisting of a halogen, a hydroxyl, a thiol, a cyano, a C₀-C₃alkyl-ethynyl (—(CH₂)₀₋₃—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

In a particular aspect, R is H, a C₁-C₆alkyl or a C₀-C₃alkyl-ethynyl (—(CH₂)₀₋₆—C≡CH), preferably H, a C₁-C₃alkyl or —CH₂—C≡CH, more preferably H, a methyl or —CH₂—C≡CH.

In a particular aspect, the compound can be selected in the group consisting of

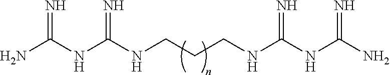

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12;

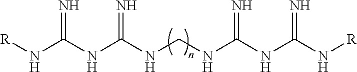

with n being an integer from 11 to 16, e.g., 11, 12, 13, 14, 15 or 16, more specifically an integer selected from 11 to 15, e.g., 11, 12, 13, 14 or 15, or an integer selected from 11 to 14, e.g., 11, 12, 13 or 14, or an integer selected from 12 to 14, e.g., 12, 13, or 14, and R being a C₁-C₆alkyl, preferably a C₂-C₆alkyl, e.g., a methyl, ethyl, propyl, butyl, pentyl or hexyl, or a C₀-C₃alkyl-ethynyl (—(CH₂)₀₋₃—C≡CH), preferably —CH₂—C≡CH;

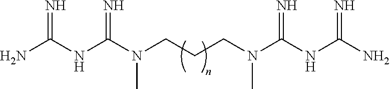

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

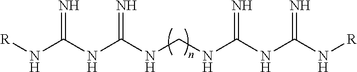

with n being an integer from 9 to 14, e.g., 9, 10, 11, 12, 13 or 14, more specifically an integer selected from 9 to 13, e.g., 9, 10, 11, 12, or 13, or an integer selected from 9 to 12, e.g., 9, 10, 11, or 12, or an integer selected from 10 to 12, e.g., 10, 11, or 12;

or a pharmaceutically acceptable salt, stereoisomer, tautomer or solvate thereof.

In another more particular aspect, the compound has a structure of formula (II), wherein the formula (II) is

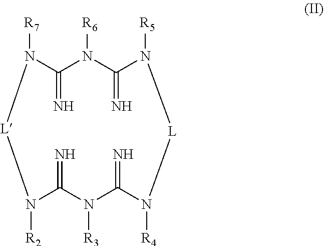

with
R₂, R₃, R₄, R₅, R₆, and R₇ being independently selected in the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;
-L- and -L′- being independently a linear hydrocarbon chain of 4 to 16 carbons optionally interrupted by
   a heteroatom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀ cycloheteroalkyl, a C₆-C₁₂aryl, and a C₅-C₁₂heteroaryl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy,

or a pharmaceutically acceptable salt, stereoisomer, tautomer or solvate thereof.

Optionally, the compounds of the present invention have a structure of formula (II), with
R₂, R₃, R₄, R₅, R₆, and R₇ being independently selected in the group consisting of H, a C₁-C₆alkyl, a C₀-C₃alkyl-ethynyl (—(CH₂)₀₋₆—C≡CH), a C₃-C₁₀cycloalkyl, and a C₃-C₁₀ cycloheteroalkyl, said alkyl, cycloalkyl, or cycloheteroalkyl, being optionally substituted by a R group or a R′ group;
-L- and -L′- being independently a linear hydrocarbon chain of 4 to 16 carbons optionally interrupted by
   a heteroatom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
   a 3-7-membered ring, optionally selected from the group consisting of a cycloalkyl and a heterocycloalkyl, said ring being optionally substituted by a group R or R′;
said linear hydrocarbon chain being optionally substituted by a group R or R′;

with R being selected from the group consisting of a C₁-C₆alkyl optionally substituted by at least one halogen, a C₁-C₆ alkyloxy optionally substituted by at least one halogen, a C₃-C₁₀cycloalkyl and a C₃-C₁₀cycloheteroalkyl, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy,

or a pharmaceutically acceptable salt, stereoisomer, tautomer or solvate thereof.

Optionally, the compounds of the present invention have a structure of formula (II), with
R₂, R₃, R₄, R₅, R₆, and R₇ being independently selected in the group consisting of H, a C₁-C₆alkyl, a C₀-C₃alkyl-ethynyl (—(CH₂)₀₋₆—C≡CH), said alkyl being optionally substituted by a R group or a R′ group;
   L- and -L′- being independently a linear hydrocarbon chain of 4 to 16 carbons optionally interrupted by
   a heteroatom; and/or
   a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—);
said linear hydrocarbon chain being optionally substituted by a group R or R′;

with R being selected from the group consisting of a C₁-C₆alkyl optionally substituted by at least one halogen, and a C₁-C₆alkyloxy optionally substituted by at least one halogen, the group being optionally substituted by a group R′, and

R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃ alkoxy, or a pharmaceutically acceptable salt, stereoisomer, tautomer or solvate thereof.

In one aspect, R₃ and R₆ are H. Optionally, R₄ and R₅ are independently selected in the group consisting of H, a C₁-C₃alkyl and a C₀-C₃alkyl-ethynyl (—(CH₂)₀₋₃—C≡CH), preferably from the group consisting of H, a methyl and —CH₂-ethynyl (—CH₂—C≡CH). Optionally, R₄ and R₅ are the same or are different.

In another aspect, R₂, R₃, R₆, and R₇ are H. Optionally, R₄ and R₅ are independently selected in the group consisting of H, a C₁-C₃alkyl and a C₀-C₃alkyl-ethynyl (—(CH₂)ₙ_₃—C≡CH), preferably from the group consisting of H, a methyl and —CH₂-ethynyl (—CH₂—C≡CH). Optionally, R₄ and R₅ are the same or are different.

In a specific aspect, R₂, R₃, R₆, and R₇ are H, R₄ and R₅ are a C₀-C₃alkyl-ethynyl (—(CH₂)₀₋₃—C≡CH) or one of R₄ and R₅ is a C₀-C₃alkyl-ethynyl (—(CH₂)₀₋₃—C≡CH) and the other is H. Preferably, R₄ and R₅ are —CH₂-ethynyl (—CH₂—C≡CH) or one of R₄ and R₅ is —CH₂-ethynyl (—CH₂—C≡CH) and the other is H.

In another specific aspect, R₂, R₃, R₆, and R₇ are H, R₄ and R₅ are a C₁-C₃alkyl or one of R₄ and R₅ is a C₁-C₃alkyl, preferably a methyl, and the other is H.

In another specific aspect, R₂, R₃, R₄, R₅, R₆, and R₇ are H.

L and L′ can be different or the same.

L and L′ are such that they allow the proper arrangement of the two biguanidyl radicals so as to form stable complex comprising the two biguanidyl radicals with one copper or iron cation.

Optionally, L and L′ are designed so as to increase the lipophilicity of the compound.

Optionally, L and L′ comprise a linear hydrocarbon chain of 4 to 16 carbons, e.g., 4, 5, 6, 7, 8, 9, 10, 11, 12, 13, 14, 15 or 16, more specifically of 6 to 15 carbons, e.g., 6, 7, 8, 9, 10, 11, 12, 13, 14 or 15, or of 7 to 14 carbons, e.g., 7, 8, 9, 10, 11, 12, 13 or 14, or of 8 to 14 carbons, e.g., 8, 9, 10, 11, 12, 13, or 14, or of 9 to 14 carbons, e.g., 9, 10, 11, 12, 13, or 14, preferably of 10 to 14 carbons, e.g., 10, 11, 12, 13, or 14.

Optionally, L and L′ comprise or consist of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being H, a group R′ or forming together a six-member ring, optionally substituted by one or two R′ groups or fused with a cycloalkyl, an aryl, a cycloheteroalkyl or a heteroaryl having 3 to 6 members, with Rₑ and R_{f} being independently H or a R′ group, “f” and “g” being an integer independently selected from 0 to 12 and the sum “f” and “g” being an integer selected from 0 to 12; or
—(CH₂)ₕ—C≡C—C≡C—(CH₂)ᵢ—, with “h” and “i” being an integer independently selected from 0 to 12 and the sum “h” and “i” being an integer selected from 0 to 12; or
—(CH₂)ₕ—C≡C—(CH₂)ᵢ—, with “h” and “i” being an integer independently selected from 0 to 14 and the sum “h” and “i” being an integer selected from 0 to 14; or
—(CH₂)ₙ—, with “n” being an integer selected from 4 to 16; or
—CRₐ—(CH₂)ₙ—CR_{b}—, with Rₐ and R_{b} being H, or a group R′ and with “n” being an integer selected from 2 to 14; or
—(CH₂)ⱼ-cycloalkyl-(CH₂)ₖ— or —(CH₂)ⱼ-heterocycloalkyl-(CH₂)ₖ—, with “j” and “k” being an integer independently selected from 0 to 12 and the sum “j” and “k” being an integer selected from 0 to 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being H, a group R′ or forming together a 4-6 member ring, optionally substituted by one or two R′ groups or fused with a cycloalkyl, an aryl, a cycloheteroalkyl or a heteroaryl having 3 to 6 members, with Rₑ and R_{f} being independently H or a R′ group, “p” and “q” being an integer independently selected from 0 to 12 and the sum “p” and “q” being an integer selected from 0 to 12;

with R′ being selected from the group consisting of a halogen, a hydroxyl, a thiol, a cyano, an ethynyl (—C≡CH), a nitro, an amino (—NH2), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆ alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, and a C₃-C₁₀cycloheteroalkyl, with R″ being H or a C₁-C₃ or C₁-C₆alkyl optionally substituted by at least one halogen and said cycloalkyl or cycloheteroalkyl being optionally substituted by a halogen, a hydroxyl, a cyano, a nitro, an amino, or a C₁-C₃alkoxy.

Optionally, R′ is selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, with R″ being H or a C₁-C₃ or C₁-C₆alkyl optionally substituted by at least one halogen.

In a specific aspect, L and L′ comprise or consist of
—(CH₂)_{f}—CRₐ═CRₑ—CR_{f}═CR_{b}—(CH₂)_{g}—, preferably —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “f” and “g” being an integer independently selected from 0 to 12 and the sum “f” and “g” being an integer selected from 0 to 12, or from 3 to 10, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 0 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 0 to 14, or from 3 to 12, or from 4 to 10, or from 5 to 10, or from 6 to 10; or
—(CH₂)ₙ—, with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, and with “n” being an integer selected from 2 to 14, e.g., 2, 3, 4, 5, 6, 7, 8, 9, 10, 11, 12, 13 or 14, more specifically an integer selected from 4 to 13, e.g., 4, 5, 6, 7, 8, 9, 10, 11, 12, or 13, or an integer selected from 5 to 12, e.g., 5, 6, 7, 8, 9, 10, 11, or 12, or an integer selected from 6 to 12, e.g., 6, 7, 8, 9, 10, 11, or 12, or an integer selected from 7 to 12, e.g., 7, 8, 9, 10, 11, or 12, preferably an integer selected from 8 to 12, e.g., 8, 9, 10, 11, or 12; or
—(CH₂)ₚ—CHR_{c}—CRₑ═CR_{f}—CHR_{d}—(CH₂)_{q}—, preferably —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, with Rₑ and R_{f} being independently H, a C₁-C₆alkyl or a C₀-C₃alkyl-ethynyl (—(CH₂)₀₋₆—C≡CH), preferably independently H, a C₁-C₃alkyl or —CH₂—C≡CH, more preferably independently H, a methyl or —CH₂—C≡CH, “p” and “q” being an integer independently selected from 0 to 12 and the sum “p” and “q” being an integer selected from 0 to 12, or from 3 to 10, or from 4 to 10, or from 5 to 10, or from 6 to 10.

In a very specific aspect, the compound is selected from the group consisting of

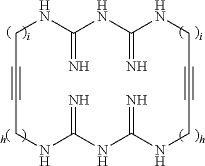

with “h” and “i” being an integer independently selected from 0 to 14 and the sum “h” and “i” being an integer selected from 0 to 14, or from 3 to 13, or from 4 to 12, or from 5 to 10, or from 6 to 10; and

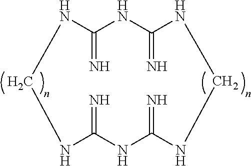

with “n” being an integer selected from 4 to 16, e.g., 4, 5, 6, 7, 8, 9, 10, 11, 12, 13, 14, 15 or 16, more specifically an integer selected from 6 to 15, e.g., 6, 7, 8, 9, 10, 11, 12, 13, 14 or 15, or an integer selected from 7 to 14, e.g., 7, 8, 9, 10, 11, 12, 13 or 14, or an integer selected from 8 to 14, e.g., 8, 9, 10, 11, 12, 13, or 14, or an integer selected from 9 to 14, e.g., 9, 10, 11, 12, 13, or 14, preferably an integer selected from 10 to 14, e.g., 10, 11, 12, 13, or 14;

or a pharmaceutically acceptable salt, stereoisomer, tautomer or solvate thereof.

In addition, the present invention relates to a new compound selected in the group consisting of

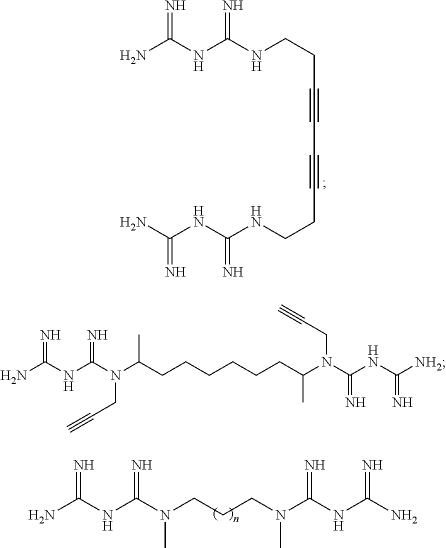

with n being an integer from 2-14 or from 4-14 or from 8-14 or from 8-12 or from 8-10; and

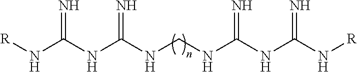

with n being an integer from 6-16, from 8-16 or from 8-12 or from 8-10 and R being an ethyl, a propyl or —CH₂—C≡CH, or a pharmaceutically acceptable salt, stereoisomer, tautomer or solvate thereof.

Optionally, in any of the specific aspect disclosed above, R, R₁, R₂, R₇ and R₈ are not aryl.

Optionally, in any of the specific aspect disclosed above, R, R₁, R₂, R₇ and R₈ are not an alkyl substituted by an aryl. Optionally, in any of the specific aspect disclosed above, R, R₁, R₂, R₇ and R₈ are not aryl or an alkyl substituted by an aryl.

Optionally, in any of the specific aspect disclosed above, L is not interrupted by an nitrogen.

Optionally, in any of the specific aspect disclosed above, n is an integer of at least 8, 9, 10, 11 or 12.

Optionally, in any of the specific aspect disclosed above, R₄ and R₅ are H.

Optionally, in any of the specific aspect disclosed above, R₁, R₂, R, and R₈ are H.

Optionally, in any of the specific aspect disclosed above, R, R₁, R₂, R₇ and R₈ are not aryl or an alkyl substituted by an aryl and L is not interrupted by an nitrogen. Optionally, R₁, R₂, R₇ and R₈ are H and L is not interrupted by an nitrogen.

Optionally, in any of the specific aspect disclosed above, R, R₁, R₂, R₇ and R₈ are not aryl or an alkyl substituted by an aryl and n is an integer of at least 8, 9, 10, 11 or 12. Optionally, R₁, R₂, R₇ and R₈ are H and n is an integer of at least 8, 9, 10, 11 or 12.

Optionally, in any of the specific aspect disclosed above, R, R₁, R₂, R₇ and R₈ are not aryl or an alkyl substituted by an aryl and R₄ and R₅ are H. Optionally, R₁, R₂, R₇ and R₈ are H and R₄ and R₅ are H.

Optionally, in any of the specific aspect disclosed above, R, R₁, R₂, R₇ and R₈ are not aryl or an alkyl substituted by an aryl, L is not interrupted by an nitrogen and n is an integer of at least 8, 9, 10, 11 or 12. Optionally, R₁, R₂, R₇ and R₈ are H, L is not interrupted by an nitrogen and n is an integer of at least 8, 9, 10, 11 or 12.

Optionally, in any of the specific aspect disclosed above, R, R₁, R₂, R₇ and R₈ are not aryl or an alkyl substituted by an aryl, L is not interrupted by an nitrogen, n is an integer of at least 8, 9, 10, 11 or 12 and R₄ and R₅ are H. Optionally, R₁, R₂, R₇ and R₈ are H, L is not interrupted by an nitrogen, n is an integer of at least 8, 9, 10, 11 or 12 and R₄ and R₅ are H.

In a preferred aspect, the compound can be selected in the group consisting of:

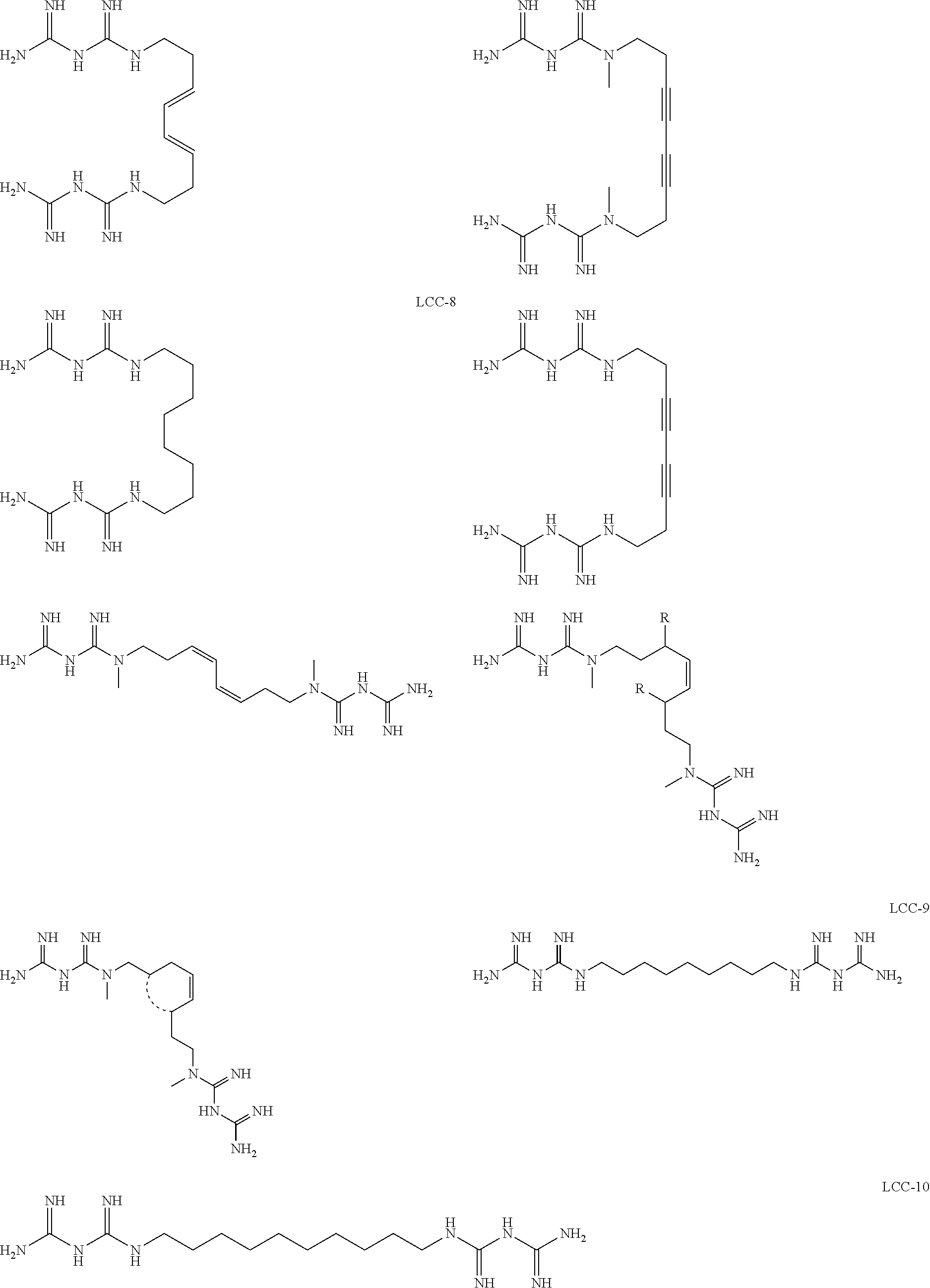

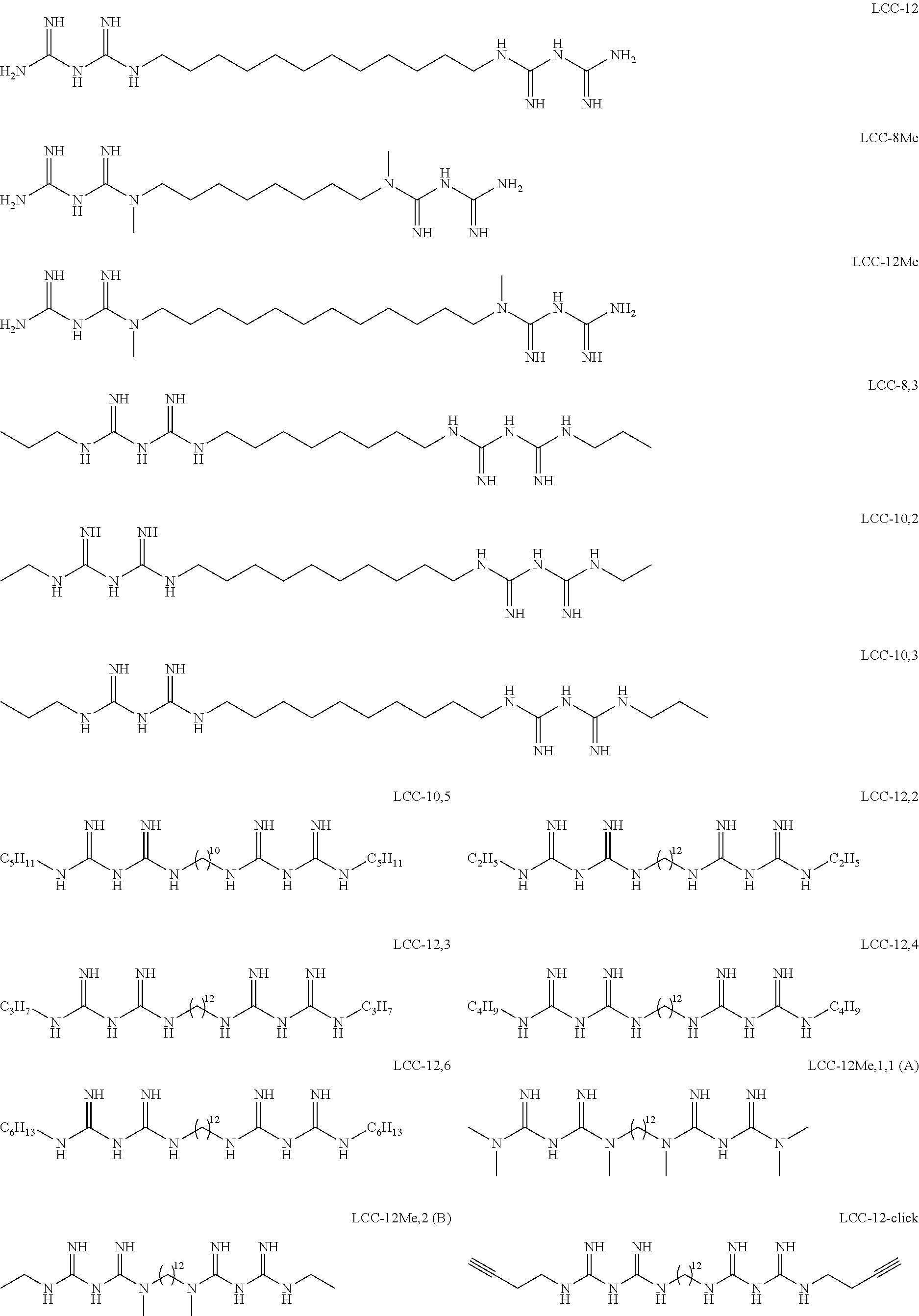

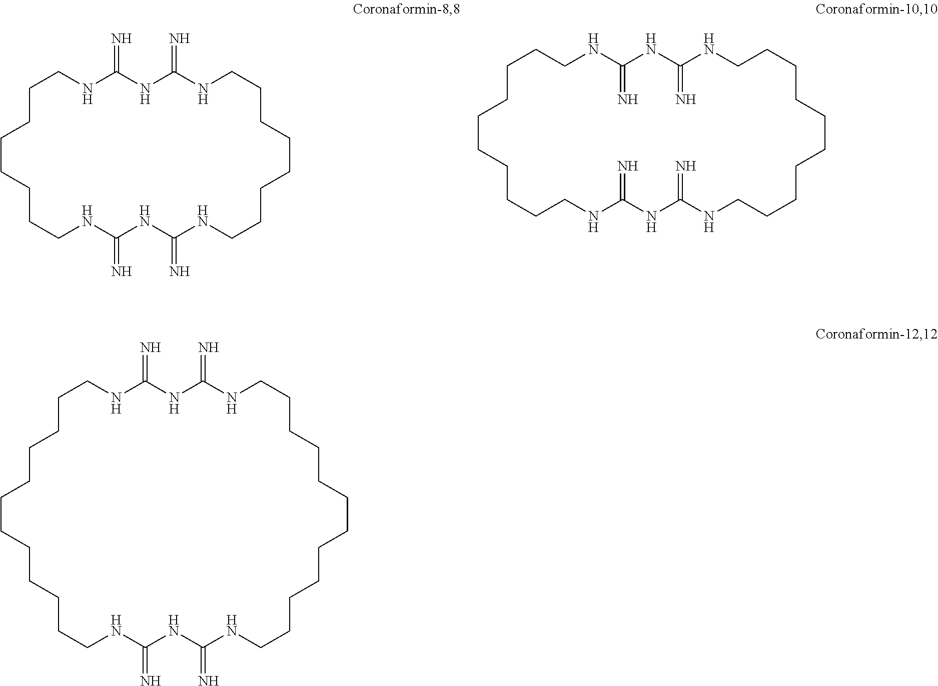 [SMILES: N=C1NCCCCCCCCNC(=N)NC(=N)NCCCCCCCCNC(=N)N1 ; N=C1NCCCCCCCCCCNC(=N)NC(=N)NCCCCCCCCCCNC(=N)N1 ; N=C1NCCCCCCCCCCCCNC(=N)NC(=N)NCCCCCCCCCCCCNC(=N)N1]

or a pharmaceutically acceptable salt, stereoisomer, tautomer or solvate thereof. Preferably, the compound is selected from the group consisting of LCC-8, LCC-9, LCC-10, LCC-12, LCC-8Me, and LCC-12Me, or a pharmaceutically acceptable salt, stereoisomer, tautomer or solvate thereof. More preferably, the compound is selected from the group consisting of LCC-10, LCC-12 and LCC-12Me, or a pharmaceutically acceptable salt, stereoisomer, tautomer or solvate thereof.

In a specific aspect, the compound is in the form of a pharmaceutically acceptable salt, in particular a di-formic acid salt or a di-hydrochloride salt.

The present invention relates to a pharmaceutical or veterinary composition comprising a new compound as disclosed herein and a new compound as disclosed herein as a drug or medicine.

Uses

Anti-Inflammatory Effects

The compounds of the present invention can be useful as anti-inflammatory agent. Indeed, as shown in the examples section, they are able to prevent the activation of macrophages, in particular with an efficiency at 1,000 fold better than Metformin (see FIG. 3). They are also able to block the activation of genes involved in the inflammation (see FIG. 4) by inhibiting their demethylation as illustrated by their capacity to decrease the amount of α-ketoglutarate or of NAD⁺ necessary for the α-ketoglutarate production (see FIGS. 3 and 11). Their improved inhibitory capacity that could be explained by the improved ability to form a complex with copper is illustrated by FIG. 3. In addition, due to the specificity of the compounds for mitochondrion, the copper chelation should be specific to mitochondrial copper, thereby avoiding to induce adverse side effects.

Accordingly, the present invention relates to a compound as disclosed herein or a pharmaceutical composition comprising it for use as anti-inflammatory agent or for use for the treatment of an inflammatory disease or disorder, and to the use of a compound as disclosed herein or a pharmaceutical composition comprising it for the manufacture of a medicament useful as anti-inflammatory agent or for the treatment of an inflammatory disease or disorder. It further relates to the treatment of a subject suffering of an inflammatory disease or disorder, comprising administering a therapeutic effective amount of a compound as disclosed herein or a pharmaceutical composition comprising it to said subject, thereby inducing an anti-inflammatory effect.

The inflammatory disease or disorder can also be selected from the group consisting of a systemic inflammatory response syndrome, a cytokine release syndrome (CRS), an Adult Respiratory Distress Syndrome (ARDS), a Macrophage Activation Syndrome (MAS), an Alveolar inflammatory response, a paediatric multisystem inflammatory syndrome, a Hemophagocytic lymphohistiocytosis (HLH), systemic lupus erythematosus, a sepsis, in particular septic shock, Crohn's disease, ulcerative colitis, rheumatoid arthritis, or a hypercytokinemia. In particular, these inflammatory diseases or disorders can be due to an infection by a bacterium, a fungus, or a virus, especially a virus of coronaviridae family, more specifically Orthocoronavirinae subfamily such as Middle East respiratory syndrome-related coronavirus (MERS-CoV), β-CoV, Severe acute respiratory syndrome coronavirus (SARS-CoV), β-CoV or Severe acute respiratory syndrome coronavirus 2 (SARS-CoV-2), β-CoV; or a virus of Orthomyxoviridae family, more particularly influenza, such as Influenza virus A, Influenza virus B, Influenza virus C; to a non-infectious disease such as graft-versus-host disease (GVHD), systemic inflammatory response syndrome (SIRS), inhalation of harmful substances, pancreatitis, pneumonia, trauma, massive blood transfusions, burns, ischemia/reperfusion, hemorrhagic shock, systemic juvenile idiopathic arthritis (SJIA) and adult Still's disease, or to a condition or acute toxicity that may occur after treatment with some types of immunotherapy, such as antibodies and adoptive T cell therapy such as CAR-T cells therapy and caused by a large and rapid release of cytokines into the blood from immune cells affected by the immunotherapy.

Alternatively, the inflammatory disease or disorder can also be selected from the group consisting of Crohn disease, inflammatory bowel disease, asthma, chronic obtrusive pulmonary disease (COPD), systemic lupus erythematosus, cystic fibrosis, psoriasis, arthritis such as infectious arthritis, and multiple sclerosis.

Autoimmune Diseases or Disorders

In addition, the present invention also relates to a compound as disclosed herein or a pharmaceutical composition comprising it for use as anti-inflammatory agent or for use for the treatment of an autoimmune disease or disorder, and to the use of a compound as disclosed herein or a pharmaceutical composition comprising it for the manufacture of a medicament for the treatment of an autoimmune disease or disorder. It further relates to the treatment of a subject suffering of an autoimmune disease or disorder, comprising administering a therapeutic effective amount of a compound as disclosed herein or a pharmaceutical composition comprising it to said subject, thereby inducing an anti-inflammatory effect. Indeed, the current strategy for treating autoimmune diseases or disorders is to reduce inflammation.

The autoimmune disease or disorder can be selected from the group consisting of Addison disease, Hemolytic Autoimmune Anemia, Anti-Glomerular Basement Membrane Disease, Anti-Neutrophil Cytoplasmic Antibody-Associated Vasculitis including Churg-Strauss Syndrome, Granulomatosis with Polyangiitis and Microscopic Polyangiitis, Antiphospholipid Syndrome, Juvenile Arthritis, Rheumatoid Arthritis including Felty Syndrome, Rheumatoid Vasculitis, Sjogren's Syndrome and Adult-Onset Still's Disease, Autoimmune Diseases of the Nervous System including Anti-N-Methyl-D-Aspartate Receptor Encephalitis, Demyelinating Autoimmune Diseases, Myasthenia Gravis, Nervous System Autoimmune Disease, Polyradiculoneuropathy, Stiff-Person Syndrome, Uveomeningoencephalitic Syndrome, and CNS Vasculitis, Autoimmune Hypophysitis, Autoimmune Lymphoproliferative Syndrome, Autoimmune Pancreatitis, Birdshot Chorioretinopathy, Dermatitis Herpetiformis, Type 1 Diabetes Mellitus, Glomerulonephritis, Graves' Disease including Graves Ophthalmopathy, Autoimmune Hepatitis, Immunoglobulin G4-Related Disease, Latent Autoimmune Diabetes in Adults, Linear IgA Bullous Dermatosis, Systemic Lupus Erythematosus including Lupus Nephritis and Central Nervous System (CNS) Lupus Vasculitis, Sympathetic Ophthalmia, Bullous Pemphigoid, Pemphigus, Autoimmune Polyendocrinopathies, Idiopathic Thrombocytopenic Purpura, and Autoimmune Thyroiditis.

COVID-19 and Severe COVID-19

The present invention also relates to a compound as disclosed herein or a pharmaceutical composition comprising it for use for the treatment of COVID-19 or Severe COVID-19, and to the use of a compound as disclosed herein or a pharmaceutical composition comprising it for the manufacture of a medicament useful for the treatment of COVID-19 or Severe COVID-19. It further relates to the treatment of a subject suffering of COVID-19 or Severe COVID-19, comprising administering a therapeutic effective amount of a compound as disclosed herein or a pharmaceutical composition comprising it to said subject.

As used herein, the term “COVID-19” or “Coronavirus disease 2019” has its general meaning in the art and refers to an infectious coronavirus disease caused by severe acute respiratory syndrome coronavirus 2 (SARS-CoV-2), a newly identified coronavirus in December 2019 in Wuhan, China. The term “COVID-19” also refers to 2019-nCoV acute respiratory disease. COVID-19 results in mild to moderate respiratory disease, but may in some cases develop into severe COVID-19.

As used herein, the term “Severe COVID-19” has its general meaning in the art and refers to COVID-19 side effect resulting in severe respiratory disease, pneumonia, viral sepsis, Cytokine Release Syndrome (CRS), Acute Respiratory Distress Syndrome (ARDS), Macrophage Activation Syndrome (MAS), multi-visceral failure syndrome caused by an enhanced inflammatory response such as kidney and lung failure, respiratory failure, arterial inflammation, myocarditis (also known as inflammatory cardiomyopathy), myocardial injury, thrombosis, venous thromboembolic event, cardiovascular diseases such as described in Han Y, Zeng H, Jiang H, Yang Y, Yuan Z, Cheng X, Jing Z, Liu B, Chen J, Nie S, Zhu J, Li F, Ma C. CSC Expert Consensus on Principles of Clinical Management of Patients with Severe Emergent Cardiovascular Diseases during the COVID-19 Epidemic. Circulation. 2020 Mar. 27. doi: 10.1161/CIRCULATIONAHA.120.047011), pulmonary embolism, neurologic toxicities, Kawasaki disease (also known as mucocutaneous lymph node syndrome) and Cutaneous manifestations of COVID-19 such as described in (Sachdeva M, Gianotti R, Shah M, Lucia B, Tosi D, Veraldi S, Ziv M, Leshem E, Dodiuk-Gad R P. Cutaneous manifestations of COVID-19: Report of three cases and a review of literature. J Dermatol Sci. 2020 Apr. 29. pii: S0923-1811(20)30149-3. doi: 10.1016/j.jdermsci.2020.04.011).

Metabolic Diseases

Metformin can be used for treating metabolic diseases. As the compounds of the present invention show a better efficiency than metformin, such compounds can be useful for the treatment of metabolic diseases.

Accordingly, the present invention relates to a compound as disclosed herein or a pharmaceutical composition comprising it for use for the treatment of a metabolic disease, and to the use of a compound as disclosed herein or a pharmaceutical composition comprising it for the manufacture of a medicament useful for the treatment of a metabolic disease. It further relates to the treatment of a subject suffering of a metabolic disease, comprising administering a therapeutic effective amount of a compound as disclosed herein or a pharmaceutical composition comprising it to said subject.

The metabolic disease can be for instance selected from the group consisting of diabetes mellitus including type 1 and type 2 diabetes mellitus, insulin resistance, hyperglycemia, hyperinsulinemia, metabolic syndrome, glucose intolerance, hypertension, NAFLD, NASH and obesity.

Cardiac or Ischemic Diseases

Metformin can be used for treating cardiac and ischemic diseases. As the compounds of the present invention show a better efficiency than metformin, such compounds can be useful for the treatment of cardiac and ischemic diseases.

Accordingly, the present invention relates to a compound as disclosed herein or a pharmaceutical composition comprising it for use for the treatment of a cardiac and ischemic disease, and to the use of a compound as disclosed herein or a pharmaceutical composition comprising it for the manufacture of a medicament useful for the treatment of a cardiac and ischemic disease. It further relates to the treatment of a subject suffering of a cardiac and ischemic disease, comprising administering a therapeutic effective amount of a compound as disclosed herein or a pharmaceutical composition comprising it to said subject.

The cardiac or ischemic condition encompasses any of vascular occlusion or constriction, insufficient blood circulation, ischemia or stroke, mini-stroke, or micro infarct, coronary artery disease, heart attack, myocardial infarction, carotid artery disease, peripheral arterial disease, critical limb ischemia, claudication, cerebrovascular disease, reduced circulation in the brain, arterial occlusive disease, hypoperfusion, atherosclerosis, arteriosclerosis, thrombosis, and embolism.

Mitochondrial Dysfunctions

The present invention further relates to a compound as disclosed herein or a pharmaceutical composition comprising it for use for the treatment of a mitochondrial dysfunction, and to the use of a compound as disclosed herein or a pharmaceutical composition comprising it for the manufacture of a medicament useful for the treatment of a mitochondrial dysfunction. It further relates to the treatment of a subject suffering of a mitochondrial dysfunction, comprising administering a therapeutic effective amount of a compound as disclosed herein or a pharmaceutical composition comprising it to said subject. The mitochondrial dysfunction can be a primary mitochondrial dysfunction and a secondary mitochondrial dysfunction.

The primary mitochondrial dysfunction is selected from the group consisting of Autosomal Dominant Optic Atrophy (ADOA), Alpers-Huttenlocher syndrome (nDNA defect), Ataxia neuropathy syndrome, (nDNA defect), Barth syndrome/Lethal Infantile Cardiomyopathy (LIC), Co-enzyme Q deficiency, complex I, complex II, complex III, complex IV and complex V deficiencies (either single deficiencies or any combination of deficiency), Chronic progressive external ophthalmoplegia (CPEO), Diabetes mellitus and deafness, Kearns-Sayre syndrome (mtDNA defect), Leukoencephalopathy with Brainstem and Spinal Cord Involvement and Lactate Elevation (LBSL-leukodystrophy), Leigh syndrome (mtDNA and nDNA defects), Leber's hereditary optic neuropathy (LHON), Luft Disease, Mitochondrial myopathy, encephalopathy, lactic acidosis, and stroke syndrome (MELAS) (mtDNA defect), Mitochondrial Enoyl CoA Reductase Protein-Associated Neurodegeneration (MEPAN), Myoclonic epilepsy with ragged red fibers (MERRF), mitochondrial recessive ataxia syndrome (MIRAS), mtDNA deletion syndrome, mtDNA Depletion syndrome, mtDNA maintenance disorders, mtDNA/RNA translation defects, Mitochondrial tRNA synthetase deficiencies, Mitochondrial Myopathy, Mitochondrial neurogastrointestinal encephalopathy syndrome (MNGIE), Neurogenic muscle weakness, ataxia, and retinitis pigmentosa (NARP), Pearson syndrome, Pyruvate dehydrogenase complex deficiency (PDCD/PDH), DNA polymerase gamma deficiency (POLG), Pyruvate carboxylase deficiency, and Thymidine kinase 2 deficiency (TK2).

The secondary mitochondrial dysfunction is selected from the group consisting of Amyotrophic Lateral Sclerosis (ALS), Alzheimer's disease (AD) and other dementias, Friedreich's ataxia (FA), Huntington's disease (HD), Motor neuron diseases (MND), N-glycanase deficiency (NGLY1), Organic acidemias, Parkinson's disease (PD) and PD-related disorders, Prion disease, Spinal muscular atrophy (SMA), Spinocerebellar ataxia (SCA), Becker muscular dystrophy, Congenital muscular dystrophies, Duchenne muscular dystrophy, Emery-Dreifuss muscular dystrophy, Facioscapulohumeral muscular dystrophy, Myotonic dystrophy, Oculopharyngeal muscular dystrophy, Charcot-Marie-Tooth disease, Congenital myopathies, Distal myopathies, Endocrine myopathies (hyperthyroid myopathy, hypothyroid myopathy), Giant axonal neuropathy, Hereditary spastic paraplegia, Inflammatory myopathies (dermatomyositis, inclusion-body myositis, polymyositis), Metabolic myopathies, Neuromuscular junction diseases: Autism, Cancer, Diabetes, Metabolic syndrome, Chronic fatigue syndrome, an inflammatory disorder, arthritis, neurodegenerative diseases and disorders and aging.

In particular, the secondary mitochondrial disorder can be due to copper overload and includes Indian childhood cirrhosis, Wilson's disease and Idiopathic infantile copper toxicosis or can be due to iron overload and includes Hereditary hemochromatosis, Juvenile Hemochromatosis, Neonatal iron storage disease, type I Tyrosinemia and Zellweger syndrome.

Polycystic Ovary Syndrome

Metformin can be used for treating a polycystic ovary syndrome. As the compounds of the present invention show a better efficiency than metformin, such compounds can be useful for the treatment of a polycystic ovary syndrome.

Accordingly, the present invention relates to a compound as disclosed herein or a pharmaceutical composition comprising it for use for the treatment of a polycystic ovary syndrome, and to the use of a compound as disclosed herein or a pharmaceutical composition comprising it for the manufacture of a medicament useful for the treatment of a polycystic ovary syndrome. It further relates to the treatment of a subject suffering of a polycystic ovary syndrome, comprising administering a therapeutic effective amount of a compound as disclosed herein or a pharmaceutical composition comprising it to said subject.

Mental Disorders

The present invention also relates to a compound as disclosed herein or a pharmaceutical composition comprising it for use for the treatment of a mental disorder, and to the use of a compound as disclosed herein or a pharmaceutical composition comprising it for the manufacture of a medicament useful for the treatment of a mental disorder. It further relates to the treatment of a subject suffering of a mental disorder, comprising administering a therapeutic effective amount of a compound as disclosed herein or a pharmaceutical composition comprising it to said subject. Said mental disorders can be selected from the group consisting of schizophrenia, anxiety disorders, mild cognitive disorder, depressive disorder, bipolar disorder, autism spectrum disorder and Fragile X syndrome.

Cancer

Accordingly, the present invention relates to a new compound as disclosed herein or a pharmaceutical composition comprising it for use as anti-tumoral agent or for use for the treatment of a cancer, and to the use of a compound as disclosed herein or a pharmaceutical composition comprising it for the manufacture of a medicament useful as anti-tumoral agent or for the treatment of a cancer. It further relates to the treatment of a subject suffering of a cancer, comprising administering a therapeutic effective amount of a compound as disclosed herein or a pharmaceutical composition comprising it to said subject, thereby inducing an anti-tumoral effect.

As used herein, the term “cancer” refers to any cancer that may affect anyone of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarninoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Kaposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Kaposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

Optionally, the cancer can be selected in the group consisting of rectal cancer, colorectal cancer, stomach cancer, head and neck cancer, thyroid cancer, cervical cancer, uterine cancer, breast cancer, in particular triple negative breast cancer, ovarian cancer, brain cancer, in particular glioblastoma and neuroblastoma, lung cancer, in particular small-cell lung cancer and non-small-cell lung cancer, skin cancer, bladder cancer, blood cancer, renal cancer, liver cancer, prostate cancer, multiple myeloma, pancreatic cancer and endometrial cancer. In a very particular aspect, the cancer is a pancreatic cancer.

In a particular aspect, the compounds of the present invention are of particular interest for targeting persister cancer cells, cancer-stem cells, cancer stem-like cells, drug-tolerant cancer cells, and therapy-resistant cancer cells, and for targeting epithelial-mesenchymal transition, targeting epithelial-mesenchymal plasticity. In particular, the compounds of the present invention can be used as inhibitor of cell plasticity in cancer, for instance by blocking epithelial-mesenchymal transition. They can be used to desensitize cancer cells to cytotoxic agents, especially those of the standard of care. Optionally, the present invention relates to a compound or pharmaceutical composition for use in the treatment of a subject having a cancer resistant or susceptible to become resistant to a cytotoxic agent. Optionally, said compound or pharmaceutical composition can be used in combination with said cytotoxic agent. It further relates to a method for reversing or decreasing or delaying a resistance of cancer cells to a cytotoxic agent in a subject having a cancer, comprising administering a therapeutic amount of a compound or a composition of the present invention to said subject, thereby reversing or decreasing or delaying the resistance to said cytotoxic agent, especially a chemotherapeutic agent.

Optionally, the compound or pharmaceutical composition is used in combination with radiotherapy and/or another drug, preferably an antitumoral drug, more preferable a drug selecting from the group consisting of chemotherapy, targeted therapy, hormonotherapy and immunotherapy such as immune checkpoint therapy.

As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

As used herein, the term “antitumor chemotherapy” or “chemotherapy” refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

As used herein, the term “hormonal therapy” refers to a cancer treatment having for purpose to block, add or remove hormones. For instance, in breast cancer, the female hormones estrogen and progesterone can promote the growth of some breast cancer cells.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atezolizumab, durvalumab and avelumab. Example of anti-CTLA-4 antibodies include, but are not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Example of inhibitors of the LAG3 receptor are described in US patent U.S. Pat. No. 5,773,578. Example of KIR inhibitor is IPH4102 targeting KIR3DL2.

As used herein, the term “radiotherapy” refers to radiation therapies including, but not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V. Fouillade C, Vozenin M C. The radiotherapy FLASH to save healthy tissues. Med Sci (Paris) 2015; 31:121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

The pharmaceutical compositions contemplated herein may include a pharmaceutically acceptable carrier in addition to the active ingredient(s). The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parental administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parental administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

The pharmaceutical or veterinary composition as disclosed herein may further comprise an additional active ingredient or drug.

Further aspects and advantages of the present invention will be described in the following examples, which should be regarded as illustrative and not limiting.

### BRIEF DESCRIPTION OF THE FIGURES

FIG. 1. CD44 mediates uptake of iron and copper in activated MDM. (A) Experimental setup to generate inflammatory MDM. Peripheral blood samples were collected from 22 donors. Pan monocytes were sorted, treated with GM-CSF to produce MDM and then activated with LPS and IFNγ to obtain act. MDM. (B) Flow cytometry of CD44 and TfR1 at the plasma membrane. (C) Flow cytometry and quantification of RhoNox-M fluorescence. n=4 donors. (D) Fluorescence microscopy of RhoNox-M and FITC-HA. Dotted lines delineate cell contours. Scale bar, 10 μm. Images representative of n=3 donors. (E) Western blot of iron homeostasis markers. Data representative of n=5 donors. (F) ICP-MS of cellular iron and copper in MDM and act. MDM. n=5 donors. (G) ICP-MS of cellular iron and copper in act. MDM supplemented with HMM-HA (0.6-1 MDa). n=7 donors. (H) Molecular structure (top) and ¹H-NMR spectra (bottom) of LMM-HA. Functional groups that can reversibly interact with copper are highlighted. (I) Fluorescence microscopy of a lysosomal copper(II) probe and FITC-HA. Dotted lines delineate cell contours in the DAPI channel. At least 30 cells were quantified per donor. Scale bar, 10 μm. n=6 donors. (J) ICP-MS of cellular iron and copper in aMDM transfected with siCtrl. or siCD44. n=7 donors. (K) ICP-MS of cellular iron and copper in aMDM treated with CD44 blocking antibody RG7356. n=7 donors. Mann-Whitney test for (C), (E), (F), (G), (J) and (K). Mean values±SEM. FITC, fluorescein isothiocyanate.

FIG. 2. Iron and copper regulate epigenetic plasticity underlying macrophage activation. (A) RNA-seq: Principal Component Analysis comparing MDM (n=5 donors) and act. MDM (n=5 donors) with BALF macrophages from moderate (n=3) and severe (n=6) COVID-19 patients and control BALF macrophages (n=4). (B) Gene Ontology of up-regulated genes in act. MDM vs MDM and sCovid vs control macrophages. (C) Volcano plots of genes in act. MDM compared to MDM, and sCOVID compared to control macrophages, illustrating genes coding for the COVID-19 macrophage inflammation signature and iron- and αKG-dependent demethylases. Dashed line, adjusted p-value=0.05. (D) Bubble plots representing GO term analysis of up-regulated genes in act. MDM vs MDM, MDM exposed to Salmonella typhimurium vs control MDM, sCovid vs moderate and control macrophages, MDM exposed to Leishmania major vs control MDM and MDM exposed to Aspergillus fumigatus vs control MDM. Three GO categories are presented “Inflammation” (1), “epigenetic” (E), and “copper-signaling pathway” (C). (E) left: Volcano plots of inflammatory genes in MDM exposed to Salmonella typhimurium vs control MDM, MDM exposed to Leishmania major vs control MDM and MDM exposed to Aspergillus fumigatus vs control MDM, illustrating inflammatory gene signatures. Dashed lines, adjusted p-value=0.05. Volcano plot of genes encoding for iron-dependent demethylases and acetyl-transferases in MDM exposed to Salmonella typhimurium vs control MDM, MDM exposed to Leishmania major vs control MDM and MDM exposed to Aspergillus fumigatus vs control MDM illustrating the epigenetic states of inflammatory macrophages. Dashed lines, adjusted p-value=0.05.

FIG. 3. Copper regulates metabolic and epigenetic plasticity in activated MDM, which can be controlled by biguanides. (A) Molecular structures of Metformin (Met), lipophilic copper clamp C₁₂ (LCC-12) and corresponding copper complexes. (B) αKG quantification. n=5 donors. (C) Copper-catalyzed oxidation of NADH by H₂O₂. Data representative of n=3 independent replicates. (D) Volcano plots of genes in act. MDM compared to MDM illustrating genes coding for the metabolic pathways leading to the production of αKG. Dashed line, adjusted p-value=0.05. (E) NAD⁺/NADH ratio measurement. n=4 donors. (F) Chemical labeling of metforminyn in cells using click chemistry. 488 represents Alexa-488; and Fluorescence microscopy (right) of labeled metforminyn co-localizing with the mitochondrial component cytochrome c (Cyt c) in act. MDM. Scale bar, 10 μm. (G) Molecular structures and HRMS of biguanides complexed with copper(II). (H) Fluorescence microscopy of H3K9me2 and H3K4me3 of a representative donor. Scale bar, 10 μm. Quantification normalized against MDM. At least 50 cells were quantified per condition. n=7 donors. (I) Schematic illustration of metals regulating plasticity and effect of biguanides. (J) Fluorescence microscopy of histone H3 and corresponding methyl marks in act. MDM of a representative donor. Scale bar, 10 μm. Quantification normalized against MDM. At least 50 cells were quantified per condition. n=4-5 donors. MDM were activated with LPS and IFNγ (act. MDM), and co-treated with Met (10 mM) or LCC-12 (10 μM) as indicated. Kruskal-Wallis test with Dunn's post-test. Mean values±SEM.

FIG. 4. Biguanide treatments lead to the production of transcriptionally distinct macrophages and improve survival of LPS-treated mice. (A) RNA-seq: Principal Component Analysis comparing MDM (n=5 donors), act. MDM (n=5 donors), act. MDM co-treated with Metformin (n=5 donors) or LCC-12 (n=5 donors). (B) Gene Ontology analysis of genes in act. MDM whose up-regulation is antagonized by biguanides. (C) Volcano plots highlighting genes representative of the COVID-19 macrophage inflammation signature in act. MDM co-treated with biguanides compared to act. MDM. Dashed line, adjusted p-value=0.05. (D) Kaplan-Meier survival curves of mice challenged with LPS (20 mg/kg/single dose, IP, n=10), co-treated with dexamethasone (10 mg/kg/single dose 1 h prior challenge, PO, n=10), or with LCC-12 (300 μg/kg/d, 2 h prior challenge, then 24 h, 48 h and 72 h post challenge, IP, n=10). Mantel-Cox Log-rank test. (E) Quantification of IL-6 to IL-10 ratio secreted by act. MDM treated with biguanides. n=6 donors. (F) Flow cytometry of CD80 and CD86 cell surface markers. n=8 donors. Kruskal-Wallis test with Dunn's post-test. Mean values±SEM.

FIG. 5. Biguanide treatments improve survival in a murine model of sepsis with cecal ligation and puncture (CLP)

(A) Kaplan-Meier survival curves of mice challenged by cecal ligation and puncture (CLP) using a 21G needle and treated with LCC-12 (0.3 mg/kg, 4 h post-challenge, IP, n=10), dexamethasone (1.0 mg/kg at to) or saline solution (IP, n=10). (B) Kaplan-Meier survival curves of mice challenged by CLP using a 25G needle and treated with LCC-12 (0.3 mg/kg, 4 h post-challenge, IP, n=10) or saline solution (IP, n=10). Mantel-Cox Log-rank test for. Hazard ratio calculated using Mantel-Haenszel. n.d.=not determined. (C) Curves showing the average body weight of mice challenged by CLP using a 25G needle and treated with LCC-12 (0.3 mg/kg, 4 h post-challenge, IP, n=10) or saline solution (IP, n=10). (D) Curves showing the average gravity score of symptoms of mice challenged by CLP using a 25G needle and treated with LCC-12 (0.3 mg/kg, 4 h post-challenge, IP, n=10) or saline solution (IP, n=10). Clinical score are out of a total of 15, with 0 representing no symptoms and 15 representing maximal symptoms. Gravity score criteria are indicated. For (C) and (D) 2-way ANOVA. Mean values±SEM.

FIG. 6: Effect of LCC-12 on other immune cells than macrophages.

(A) CD4⁺ T cells, non-activated (naCD4) and activated (aCD4). Left: Flow cytometry of CD44 at the plasma membrane. Right: Flow cytometry of CD25 and CD69 cell surface markers of naCD4, aCD4 and aCD4 treated with LCC-12 (10 μM). (B) CD8⁺ T cells, non-activated (naCD8) and activated (aCD8). Left: Flow cytometry of CD44 at the plasma membrane. Right: Flow cytometry of CD25 and CD69 cell surface markers of naCD8, aCD8 and aCD8 treated with LCC-12 (10 μM). (C) Neutrophils, non-activated (naG) and activated (aG). Left: Flow cytometry of CD44 at the plasma membrane. Right: Flow cytometry of CD64 and CD66b cell surface markers of naG, aG and aG treated with LCC-12 (10 μM). (D) Monocytes, non-activated (naMo) and activated (aMo). Left: Flow cytometry of CD44 at the plasma membrane. Right: Flow cytometry of CD25 and CD80 cell surface markers of naMo, aMo and aMo treated with LCC-12 (10 μM). (E) Dendritic cells, non-activated (naDC) and activated (aDC). Left: Flow cytometry of CD44 at the plasma membrane. Right: Flow cytometry of CD40, CD83, CD80 and CD86 cell surface markers of naDC, aDC and aDC treated with LCC-12 (10 μM).

FIG. 7. Biguanides preferentially target cancer cells in the mesenchymal cell state with favorable IC₅₀ values compared to the standard of care

Cell viability curves of cells treated with 10 μM LCC-12 on cell lines as indicated. Cells were co-treated with TGF-β or OSM as indicated.

FIG. 8. Biguanides block cell plasticity in cancer cells undergoing epithelial-to-mesenchymal transition

(A) Flow cytometry of CD44 surface staining of cells treated with TGF-β or OSM. (B) Box plots of ICP-MS of cells treated with TGF-β or OSM, showing increase of copper in the mesenchymal state. Mann-Whitney test. (C) Western blot of cells treated with TGF-β or OSM, showing increase of SOD2 in the mesenchymal state. (D) Western blot of mesenchymal and epithelial markers of cells treated with TGF-β or OSM, showing that LCC-12 blocks EMT.

FIG. 9. Biguanides show efficacy on biopsy-derived organoids of pancreatic ductal adenocarcinoma (PDAC)

Chemograms of biopsy-derived organoids of pancreatic ductal adenocarcinoma treated with LCC-12 with the IC₅₀-values indicated.

FIG. 10. LCC-12 targets copper(II) in mitochondria

(A) Molecular structure of isotopologue ¹⁵N-¹³C-LCC-12. (B) NanoSIMS images of ¹⁵N and ¹⁹⁷Au in aMDM. Scale bar, 10 μm. (C) Schematic illustration of click labeling of alkyne-containing LCC-12 in cells. (D) Fluorescence microscopy of labeled LCC-12 (0.1 μM) in activated MDM (aMDM), showing localization in vicinity of cytochrome c (cyt c) in mitochondria. n=6 donors. 50 cells were quantified per donor. (E) Fluorescence microscopy of labeled LCC-12 (0.1 μM) in aMDM co-treated with carbonyl cyanide m-chlorophenyl hydrazone (CCCP). Scale bar, 10 μm. (F) Fluorescence microcopy images of labeled LCC-12 (0.1 μM). Click labeling performed in the presence of ascorbate without added copper(II) in naMDM and aMDM. Scale bar, 10 μm. (G) Fluorescence microcopy images of labeled LCC-12 (0.1 μM). Click labeling performed with and without ascorbate (Asc) in absence of added copper(II) in aMDM. Scale bar, 10 μm. (H) Schematic illustration of mitochondrial isolation and ICP-MS of mitochondrial copper in naMDM and aMDM. n=6 donors. (E)-(G) Student's T-test. Mean values±SEM. Images of a representative donor are shown and at least 50 cells were quantified per condition. (H) Mann-Whitney test. Mean values±SEM.

FIG. 11. LCC-12 targets mitochondrial metabolism

(A) Reaction scheme of H₂O₂ biosynthesis from superoxide catalyzed by mitochondrial superoxide dismutase 2 (SOD2). (B) Fluorescence microcopy images of SOD2 in non-activated MDM (naMDM) and activated MDM (aMDM). Mitochondria were stained using an antibody against cyt c. Student's T-test. Mean values±SEM. Images of a representative donor are shown and at least 50 cells were quantified per condition. (C) Flow cytometry analysis of mitochondrial H₂O₂ in naMDM and aMDM. n=6 donors. (D) and (E) Box plots of quantitative mass-spectrometry-based metabolomics of NAD⁺, NADH, αKG and acetyl-CoA. (F) Heatmap of quantitative mass-spectrometry-based metabolomics of total cellular extracts of key metabolites whose biosynthesis depend on NAD(H). n=9 donors. MDM were activated with LPS and IFNγ (act. MDM), and co-treated with Met (10 mM) or LCC-12 (10 μM) as indicated. Kruskal-Wallis test with Dunn's post-test. Mean values±SEM.

### EXAMPLES

### Example 1: Synthesis of Compounds

Products were purified on a preparative HPLC Quaternary Gradient 2545 equipped with a Photodiode Array detector (Waters) fitted with a reverse phase column (XBridge Prep C18 5 μm OBD 30×150 mm). NMR spectroscopy was performed on Bruker spectrometers. Spectra were run in DMSO-d₆ or Methanol-d₆ at 298 K unless stated otherwise. ¹H-NMR spectra were recorded at 400 or 500 MHz, and chemical shifts δ are expressed in ppm using the residual non-deuterated solvent signal as internal standard. The following abbreviations are used: s, singlet; brs, broad signal; d, doublet; dd, doublet of doublet; t, triplet; m, multiplet; ex, exchangeable. ¹³C-NMR spectrum was recorded at 100.6 or 125.8 MHz, and chemical shifts δ are expressed in ppm using deuterated solvent signal as internal standard. The purity of final compounds, determined to be >95% by UPLC-MS, and low-resolution mass spectra (LRMS) were recorded on a Waters Acquity H-class equipped with a Photodiode array detector and SQ Detector 2 (UPLC-MS) fitted with a reverse phase column (Aquity UPLC® BEH C_{18 1.7} μm, 2.1×50 mm). High-resolution mass spectra (HRMS) were recorded on a Thermo Fisher Scientific Q-Exactive Plus equipped with a Robotic TriVersa NanoMate Advion.

General Procedures for LCC:

General procedure 1: Dicyandiamide (2.4 eq.), and the selected diamine (1, eq.) and CuCl₂ (1 eq.) were suspended in a sealed tube in water (0.4 mL/mmol) and stirred for 1 h, then heated at 80° C. for 48 h. The resulting pink mixture was filtrated, and the solid was resuspended in water (10 mL). H₂S, generated from dropwise addition of 37% aqueous (aq.) HCl on FeS (≃100 mesh powder), was passed into the mixture until it turned black. The black mixture was filtrated, and the filtrate was acidified to pH=5 with an aq. solution (soln.) of 1 M HCl. The solvent was evaporated under reduced pressure. LCC was purified with preparative HPLC (H2O/CH₃CN/Formic acid, 100:0:0.1 to 0:100:0.1) to give the LCC di-formic acid salt as a white powder.

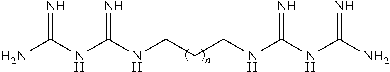

LCC-4 with n being 2; LCC-8 with n being 6; LCC-9 with n being 7; LCC-10 with n being 8; and LCC-12 with n being 10.

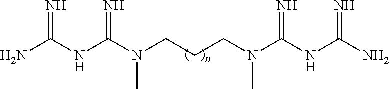

LCC-8 dimethyl with n being 6; LCC-12dimethyl with n being 10.

LCC-4 di-formic acid salt: (21%)¹H-NMR (500 MHz, DMSO-d₆) δ: 7.57 (s, 2H, ex), 7.41-6.41 (brs, 12H, ex), 3.06 (brs, 4H), 1.46 (brs, 4H) ppm. ¹³C-NMR (125.8 MHz, DMSO-d₆) δ: 159.9, 159.0, 40.7, 26.5 ppm. HRMS (ESI+) m/z: calculated for C₈H₂₂N₁₀ [M+2H]²⁺ 129.1009, found 129.1015.

LCC-8 di-formic acid salt (11%): ¹H-NMR (500 MHz, DMSO-d₆) δ: 8.68-7.96 (brs, 2H, ex), 8.44 (s, 2H, formate), 7.50-6.76 (brs, 12H, ex), 3.09 (brs, 4H), 1.43 (brs, 4H), 1.34-1.19 (m, 8H) ppm. ¹³C-NMR (125.8 MHz, DMSO-d₆) δ: 167.2 (formate), 160.2, 159.3, 41.3, 29.4, 29.1, 26.7 ppm. HRMS (ESI+) m/z: calculated for C₁₂H₃₀N₁₀ [M+2H]²⁺ 157.1322, found 157.1328.

LCC-9 di-formic acid salt (14%): ¹H-NMR (500 MHz, DMSO-d₆) δ: 8.44 (s, 2H, formate), 8.26 (brs, 2H, ex), 7.14 (brs, 12H, ex), 3.06 (t, J=6.2 Hz, 4H), 1.43 (m, 4H), 1.26 (brs, 10H) ppm. ¹³C-NMR (125.8 MHz, DMSO-d₆) δ: 167.0 (formate), 160.2, 159.3, 41.4, 29.4 (2C), 29.2, 26.8 ppm. HRMS (ESI+) m/z calculated for C₁₃H₃₂N₁₀ [M+2H]²⁺ 164.1400, found 164.1400.

LCC-10 di-formic acid salt: (13%)¹H-NMR (500 MHz, DMSO-d₆) δ: 8.88-7.96 (brs, 2H, ex), 8.47 (s, 2H, formate), 7.43-6.88 (brs, 12H, ex), 3.05 (t, J=6.8 Hz, 4H), 1.43 (m, 4H), 1.34-1.16 (m, 12H) ppm. ¹³C-NMR (125.8 MHz, DMSO-d₆) δ: 166.6 (formate), 160.0, 159.0, 41.1, 29.1 (2C), 28.9, 26.5 ppm. HRMS (ESI+) m/z: calculated for C₁₄H₃₄N₁₀ [M+2H]²⁺ 171.1478, found 171.1485.

LCC-12 di-formic acid salt: (24%). ¹H-NMR (500 MHz, DMSO-d₆) δ: 8.80-8.08 (brs, 2H, ex), 8.47 (s, 2H, formate), 7.60-6.78 (brs, 12H, ex), 3.05 (brs, 4H), 1.43 (brs, 4H), 1.32-1.18 (m, 16H) ppm. ¹³C-NMR (125.8 MHz, DMSO-d₆) δ: 167.2 (formate), 160.4, 159.3, 41.3, 29.5 (3C), 29.3, 26.8 ppm. HRMS (ESI+) m/z: calculated for C₁₆H₃₈N₁₀ [M+2H]²⁺ 185.1635, found 185.1635.

LCC-12 dimethyl di-formic acid salt: (18%)¹H-NMR (500 MHz, DMSO-d₆) δ: 8.47 (s, 2H, formate), 7.70-6.90 (brs, 12H, ex), 3.29 (t, J=7.5 Hz, 4H), 2.90 (s, 6H), 1.48 (m, 4H), 1.32-1.16 (m, 16H) ppm. ¹³C-NMR (125.8 MHz, DMSO-d₆) δ: 166.7 (formate), 159.9, 158.7, 49.8, 35.8, 29.5 (2C), 29.3, 27.4, 26.5 ppm. HRMS (ESI+) m/z: calculated for C₁₈H₄₂N₁₀ [M+2H]²⁺ 199.1791, found 199.1798.

LCC-8 dimethyl di-formic acid salt: (14%)¹H-NMR (500 MHz, DMSO-d₆) δ: 8.44 (s, 2H, formate), 7.62-6.73 (m, 12H, ex), 3.29 (t, J=7.5 Hz, 4H), 2.90 (s, 6H), 1.48 (m, 4H), 1.30-1.17 (m, 8H) ppm. ¹³C-NMR (125.8 MHz, DMSO-d₆) δ: 166.4 (formate), 159.8, 158.8, 49.8, 35.9, 29.3, 27.4, 26.4 ppm. HRMS (ESI+) m/z: calculated for C₁₄H₃₄N₁₀ [M+2H]²⁺ 171.1479, found 171.1484.

General procedure 2: Selected diamine dihydrochloride (1 eq.) was reacted with sodium di-cyanamide (2 eq.) in butanol (1.4 mL/mmol) at 130° C. overnight. After cooling to rt, the solvent was evaporated under reduced pressure and the crude was washed successively with EtOH and cold water. The bis(cyanoguanidino)alcane di-hydrochloride compound was recrystallized from H₂O/EtOH or from H₂O/ethoxyethanol to afford intermediates as white solids

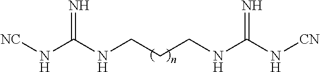

1,6-Bis(cyanoguanidino)hexane: (66%) NMR is consistent with published procedure (Gräberet al. (2013), Angew. Chem. Int. Ed., 52: 4487-4491). Recrystallized from H₂O/EtOH. ¹H-NMR (500 MHz, DMSO-d₆) δ: 7.20-6.23 (m, 6H, ex), 3.02 (brs, 4H), 1.40 (brs, 4H), 1.32-1.16 (m, 4H) ppm. ¹³C-NMR (125.8 MHz, DMSO-d₆) δ: 161.6, 118.8, 41.1, 29.3, 26.4 ppm.

1,8-Bis(cyanoguanidino)octane: (62%) NMR is consistent with published procedure.¹ Recrystallized from H₂O/EtOH. ¹H-NMR (500 MHz, DMSO-d₆) δ: 7.40-6.14 (m, 6H, ex), 2.93 (m, 4H), 1.31 (m, 4H), 1.23-1.08 (m, 8H) ppm. ¹³C-NMR (125.8 MHz, DMSO-d₆) δ: 161.6, 118.9, 41.2, 29.4, 29.2, 26.6 ppm.

1,10-Bis(cyanoguanidino)decane: (32%) Recrystallized from H₂O/ethoxyethanol. ¹H-NMR (400 MHz, DMSO-d₆) δ: 7.66-5.92 (m, 6H, ex), 3.03 (m, 4H), 1.40 (m, 4H), 1.34-1.16 (m, 12H) ppm. ¹³C-NMR (100.6 MHz, DMSO-d₆) δ: 161.7, 118.9, 41.2, 29.4 (2C), 29.2, 26.7 ppm.

1,12-Bis(cyanoguanidino)dodecane: (44%) Recrystallized from H₂O/ethoxyethanol. ¹H-NMR (400 MHz, DMSO-d₆) δ: 7.21-6.18 (m, 6H, ex), 3.03 (m, 4H), 1.40 (m, 4H), 1.33-1.14 (m, 16H) ppm. ¹³C-NMR (100.6 MHz, DMSO-d₆) δ: 161.6, 118.9, 41.0, 29.4 (3C), 29.2, 26.7 ppm.

1,12-Bis(cyanoguanidino)dodecane dimethyl: (86%) No recrystallized. Washing were enough. ¹H-NMR (500 MHz, DMSO-d₆) δ: 7.22-6.55 (m, 4H, ex), 3.25 (m, 4H), 2.86 (s, 6H), 1.43 (brs, 4H), 1.38-1.04 (m, 16H) ppm. ¹³C-NMR (125.8 MHz, DMSO-d₆) δ: 160.9, 118.9, 49.6, 35.8, 29.4, 29.4, 29.3, 27.3, 26.4 ppm.

General procedure 3: Bis(cyanoguanidino)alcane di-hydrochloride (1 eq.) and the corresponding primary amine (2 eq.) were mixed together in a sealed tube and heated at 150° C. for 4 to 6 h without solvent. After cooling to rt, the mixture was taken up in ethanol and a large excess of ethyl acetate was added. The white precipitate was filtered off and washed with ethyl acetate to afford LCC-n,m as hydrochloride white salts.

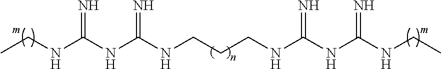

LCC-6,2 with n being 4 and m being 1; LCC-6,3 with n being 4 and m being 2; LCC-6,4 with n being 4 and m being 3; LCC-6,5 with n being 4 and m being 4; LCC-8,3 with n being 6 and m being 2; LCC-10,2 with n being 8 and m being 1; LCC-10,3 with n being 8 and m being 2; LCC-10,5 with n being 8 and m being 4; LCC-12,2 with n being 10 and m being 1; LCC-12,3 with n being 10 and m being 2; LCC-12,4 with n being 10 and m being 3; LCC-12,6 with n being 10 and m being 5.

LCC-6,2 di-hydrochloride salt: (20%)¹H-NMR (500 MHz, DMSO-d₆) δ: 7.63-6.37 (brs, 12H, ex), 3.16-3.04 (m, 8H), 1.50-1.39 (m, 4H), 1.34-1.26 (m, 4H), 1.06 (t, J=7.2 Hz, 6H) ppm. ¹³C-NMR (125.8 MHz, DMSO-d₆) δ: 158.8, 41.3, 36.3, 29.3, 26.5, 15.0 ppm. HRMS (ESI+) m/z: calculated for C₁₄H₃₄N₁₀ [M+2H]²⁺ 171.1479, found 171.1485.

LCC-6,3 di-hydrochloride salt: (45%)¹H-NMR (400 MHz, DMSO-d₆) δ: 7.82-6.28 (brs, 12H, ex), 3.16-2.98 (m, 8H), 1.53-1.40 (m, 8H), 1.35-1.25 (m, 4H), 0.87 (t, J=7.5 Hz, 6H) ppm. ¹³C-NMR (100.6 MHz, DMSO-d₆) δ: 158.8, 43.2, 41.2, 29.4, 26.5, 22.7, 11.8 ppm. HRMS (ESI+) m/z: calculated for C₁₆H₃₈N₁₀ [M+2H]²⁺ 185.1635, found 185.1641.

LCC-6,4 di-hydrochloride salt: (21%)¹H-NMR (400 MHz, DMSO-d₆) δ: 7.90-6.30 (brs, 12H, ex), 3.14-3.04 (m, 8H), 1.53-1.37 (m, 8H), 1.37-1.23 (m, 8H), 0.89 (t, J=7.2 Hz, 6H) ppm. ¹³C-NMR (100.6 MHz, DMSO-d₆) δ: 158.8, 41.2, 41.0, 31.5, 29.4, 26.5, 20.0, 14.1 ppm. HRMS (ESI+) m/z: calculated for C₁₈H₄₂N₁₀ [M+2H]²⁺ 199.1792, found 199.1798.

LCC-6,5 di-hydrochloride salt: (23%)¹H-NMR (500 MHz, DMSO-d₆) δ: 7.68-6.47 (m, 12H, ex), 3.08 (brs, 8H), 1.52-1.40 (m, 8H), 1.32-1.23 (m, 12H), 0.88 (t, J=7.1 Hz, 6H) ppm. ¹³C-NMR (125.8 MHz, DMSO-d₆) δ: 159.3, 41.2 (2C), 28.9 (3C), 26.5, 22.3, 14.4 ppm. HRMS (ESI+) m/z: calculated for C₂₀H₄₆N₁₀ [M+2H]²⁺ 213.1948, found 213.1954.

LCC-8,3 di-hydrochloride salt: (75%)¹H-NMR (400 MHz, DMSO-d₆) δ: 7.70-6.47 (m, 12H, ex), 3.13-3.04 (m, 8H), 1.52-1.39 (m, 8H), 1.33-1.23 (m, 8H), 0.87 (t, J=7.1 Hz, 6H) ppm. ¹³C-NMR (125.8 MHz, DMSO-d₆) δ: 159.1, 43.1, 41.3, 29.4, 29.2, 26.7, 22.7, 11.8 ppm. HRMS (ESI+) m/z: calculated for C₁₈H₄₂N₁₀ [M+2H]²⁺ 199.1792, found 199.1799.

LCC-10,2 di-formic acid salt. Second purification by preparative HPLC equipped with C18-reverse phase column (H₂O/CH₃CN/formic acid 95:5:0.1 to 0:100:0.1) to afford a white solid. (46%). ¹H-NMR (500 MHz, DMSO-d₆) δ: 8.49 (s, 2H, formate), 8.76-7.88 (brs, 4H, ex), 7.33-6.85 (m, 8H, ex), 3.16-2.99 (m, 8H), 1.44 (brs, 4H), 1.25 (brs, 12H), 1.05 (t, J=6.9 Hz, 6H) ppm. ¹³C-NMR (125.8 MHz, DMSO-d₆) δ: 167.4, 159.1, 41.2, 36.1, 29.4 (2C), 29.2, 26.8, 15.1 ppm. HRMS (ESI+) m/z: calculated for C₁₈H₄₂N₁₀ [M+2H]²⁺ 199.1792, found 199.1793.

LCC-10,3 di-hydrochloride salt. (75%). ¹H-NMR (400 MHz, DMSO-d₆) δ: 7.82-6.17 (m, 12H, ex), 3.07 (brs, 8H), 1.58-1.37 (m, 8H), 1.26 (brs, 12H), 0.87 (t, J=7.2 Hz, 6H) ppm. ¹³C-NMR (100.6 MHz, DMSO-d₆) δ: 159.1, 43.2, 41.4, 29.5 (2C), 29.2, 26.8, 22.8, 11.9 ppm. HRMS (ESI+) m/z: calculated for C₂₀H₄₆N₁₀ [M+2H]²⁺ 213.1948, found 213.1948.

LCC-10,5 di-formic acid salt. Second purification by preparative HPLC equipped with C18-reverse phase column (H₂O/CH₃CN/formic acid 80:20:0.1 to 0:100:0.1) afford a white solid. (35%)¹H-NMR (400 MHz, DMSO-d₆) δ: 8.48-6.55 (m, 12H, ex), 3.17-2.98 (brs, 8H), 1.51-1.35 (m, 8H), 1.35-1.18 (m, 20H), 0.87 (t, J=7.0 Hz, 6H) ppm. ¹³C-NMR (100.6 MHz, DMSO-d₆) δ: 167.7 (formate), 159.0, 41.2 (2C), 29.5 (2C), 29.2 (2C), 29.0, 26.8, 22.3, 14.4 ppm. HRMS (ESI+) m/z: calculated for C₂₄H₅₄N₁₀ [M+2H]²⁺ 241.2261, found 241.2260.

LCC-12,2 di-formic acid salt. The product was purified by preparative HPLC equipped with C18-reverse phase column (H₂O/CH₃CN/formic acid 100:0:0.1 to 50:50:0.1) to afford a white solid. (26%)¹H-NMR (400 MHz, DMSO-d₆) δ: 8.50 (s, 2H, formate), 8.80-8.04 (brs, 4H, ex), 7.41-6.85 (m, 8H, ex), 3.18-2.97 (m, 8H), 1.44 (brs, 4H), 1.26 (brs, 16H), 1.05 (t, J=6.9 Hz, 6H) ppm. ¹³C-NMR (100.6 MHz, DMSO-d₆) δ: 167.4, 159.0, 41.2, 36.1, 29.4 (3C), 29.2, 26.8, 15.1 ppm. HRMS (ESI+) m/z: calculated for C₂₀H₄₆N₁₀ [M+2H]²⁺ 213.1948, found 213.1948.

LCC-12,3 di-formic acid salt. The product was purified by preparative HPLC equipped with C18-reverse phase column (H₂O/CH₃CN/formic acid 95:5:0.1 to 0:100:0.1) to afford a white solid. (20%). ¹H-NMR (500 MHz, DMSO-d₆) δ: 8.50 (s, 2H, formate), 8.57-8.18 (brs, 4H, ex), 7.33-6.88 (m, 8H, ex), 3.05 (m, 8H), 1.52-1.38 (brs, 8H), 1.25 (brs, 16H), 0.86 (t, J=7.4 Hz, 6H) ppm. ¹³C-NMR (125.8 MHz, DMSO-d₆) δ: 167.4, 159.1, 43.1, 41.3, 29.5 (3C), 29.2, 26.8, 22.8, 11.8 ppm. HRMS (ESI+) m/z: calculated for C₂₂H₅₀N₁₀ [M+2H]²⁺ 227.2104, found 227.2105.

LCC-12,4 Second purification on preparative HPLC equipped with C18-reverse phase column (H₂O/CH₃CN/formic acid 95:5:0.1 to 0:100:0.1) to afford a white solid. (39%). ¹H-NMR (500 MHz, DMSO-d₆) δ: 8.49 (s, 2H, formate), 8.73-7.85 (brs, 4H, ex), 7.39-6.93 (brs, 8H, ex), 3.05 (m, 8H), 1.49-1.38 (m, 8H), 1.35-1.20 (brs, 20H), 0.87 (t, J=7.5 Hz, 6H) ppm. ¹³C-NMR (125.8 MHz, DMSO-d₆) δ: 167.4, 159.0, 41.3, 40.9, 31.6, 29.5 (3C), 29.2, 26.8, 20.0, 14.1 ppm. HRMS (ESI+) m/z: calculated for C₂₄H₅₄N₁₀ [M+2H]²⁺ 241.2261, found 241.2262.

LCC-12,6 Second purification on preparative HPLC equipped with C18-reverse phase column (H₂O/CH₃CN/formic acid 95:5:0.1 to 0:100:0.1) afford a white solid. (14%)¹H-NMR (500 MHz, DMSO-d₆) δ: 8.48 (s, 2H, formate), 8.30-7.40 (brs, 4H, ex), 7.40-6.70 (brs, 8H, ex), 3.06 (brs, 8H), 1.50-1.37 (m, 8H), 1.35-1.17 (m, 28H), 0.86 (t, J=7.1 Hz, 6H) ppm. ¹³C-NMR (125.8 MHz, DMSO-d₆) δ: 167.5 (formate), 158.8, 41.3 (2C), 31.5, 29.5 (4C), 29.2, 26.8, 26.5, 22.6, 14.3 ppm. HRMS (ESI+) m/z: calculated for C₂₈H₆₂N₁₀ [M+2H]²⁺ 269.2574, found 269.2572.

LCC-12,4, alcyne di-formic acid salt. Second purification on preparative HPLC equipped with C18-reverse phase column (H₂O/CH₃CN/formic acid 95:5:0.1 to 0:100:0.1) afford a white solid. (30%). ¹H-NMR (500 MHz, DMSO-d₆) δ: 9.05-7.60 (brs, 4H, ex), 8.47 (s, 2H, formate), 7.60-6.80 (brs, 8H, ex), 3.21 (t, J=6.2 Hz, 4H), 3.06 (m, 4H), 2.84 (brs, 2H), 2.34 (m, 4H), 1.44 (brs, 4H), 1.25 (brs, 16H) ppm. ¹³C-NMR (125.8 MHz, DMSO-d₆) δ: 167.6 (formate), 159.7, 158.4, 82.6, 72.7 41.3, 40.3, 29.5 (3C), 29.2, 26.8, 19.4 ppm. HRMS (ESI+) m/z: calculated for C₂₄H₄₆N₁₀ [M+2H]²⁺ 237.1948, found 237.1947.

LCC-12-dimethyl,1,1 di-formic acid salt. Second purification on preparative HPLC equipped with C18-reverse phase column (H₂O/CH₃CN/formic acid 95:5:0.1 to 0:100:0.1) afford a white solid. (41%). ¹H-NMR (500 MHz, DMSO-d₆) δ: 8.53 (s, 2H, formate), 7.31-6.87 (brs, 8H, ex), 3.28 (t, J=7.4 Hz, 4H), 2.92 (s, 12H), 2.90 (s, 6H), 1.48 (m, 4H), 1.33-1.12 (brs, 16H) ppm. ¹³C-NMR (125.8 MHz, DMSO-d₆) δ: 165.8 (formate), 158.8, 158.1, 49.8, 37.8 (2C), 35.8, 29.5 (2C), 29.3, 27.4, 26.5 ppm. HRMS (ESI+) m/z: calculated for C₂₂H₅₀N₁₀ [M+2H]²⁺ 227.2104, found 227.2103.

LCC-12-dimethyl,2 di-formic acid salt. Second purification on preparative HPLC equipped with C18-reverse phase column (H₂O/CH₃CN/formic acid 95:5:0.1 to 0:100:0.1) afford a white solid. (36%). ¹H-NMR (500 MHz, DMSO-d₆) δ: 9.30-8.67 (brs, 2H, ex), 8.51 (s, 2H, formate), 7.62-6.81 (m, 8H, ex), 3.30 (t, J=7.2 Hz, 4H), 3.08 (m, 4H), 2.91 (s, 6H), 1.49 (m, 4H), 1.33-1.15 (brs, 16H), 1.04 (t, J=7.0 Hz, 6H) ppm. ¹³C-NMR (125.8 MHz, DMSO-d₆) δ: 167.0 (formate), 158.7, 158.4, 49.9, 35.9, 35.8, 29.5 (2C), 29.3, 27.4, 26.5, 15.2 ppm. HRMS (ESI+) m/z: calculated for C₂₂H₅₀N₁₀ [M+2H]²⁺ 227.2104, found 227.2106.

Coronaformin-8,8 di-formic, di-ammonium salt. 1,8-Bis(cyanoguanidino)octane (100 mg, 0.36 mmol) and 1,8-diaminooctane dihydrochloride (51.8 mg, 0.36 mmol) were dissolved in DMSO (200 μL) followed by addition of aq. HCl (37%, 0.3 mL) and heated at 160° C. overnight. After cooling the resulting brown mixture to rt, the solvent was evaporated under high vacuum. The product was purified by preparative HPLC equipped with a C18-reverse phase column (H₂O/CH₃CN/formic acid 95:5:0.1 to 0:100:0.1) to afford a brown solid. (13%)¹H-NMR (500 MHz, DMSO-d₆) δ: 8.43 (s, 2H, formate), 8.40-8.13 (brs, 4H, ex), 7.90-7.03 (brs, 16H, ex), 3.06 (brs, 8H), 1.46 (brs, 8H), 1.28 (brs, 16H). ¹³C-NMR (125.8 MHz, DMSO-d₆) δ: 168.0 (formate), 157.6, 41.0, 29.0, 28.9, 26.4 ppm. HRMS (ESI+) m/z: calculated for C₂₀H₅₂N₁₂ [M+2NH₄+2H]⁴⁺ 115.1104, found 115.1108.

Coronaformin-10,10 di-formic, di-ammonium salt. 1,10-Bis(cyanoguanidino)decane (100 mg, 0.33 mmol) and 1,10-diaminodecane dihydrochloride (113 mg, 0.66 mmol) were dissolved in DMSO (200 μL) followed by addition of aq. HCl (37%, 2.7 mL) and heated at 160° C. overnight. After cooling the resulting brown mixture to rt, the solvent was evaporated under high vacuum. The product was purified by preparative HPLC equipped with a C18-reverse phase column (H₂O/CH₃CN/formic acid 95:5:0.1 to 0:100:0.1) to afford a brown solid. (24%)¹H-NMR (500 MHz, DMSO-d₆) δ: 8.42 (s, 2H, formate), 8.34 (brs, 4H, ex), 7.75-7.27 (brs, 16H, ex), 3.05 (m, 8H), 1.45 (brs, 8H), 1.26 (brs, 24H) ppm. ¹³C-NMR (125.8 MHz, DMSO-d₆) δ: 167.9 (formate), 157.8, 41.1, 29.3, 29.0, 28.9, 26.5 ppm. HRMS (ESI+) m/z: calculated for C₂₄H₆₀N₁₂ [M+2NH₄+2H]⁴⁺ 129.1260, found 129.1262.

Coronaformin-12,12 di-formic, di-ammonium salt. 1,12-Bis(cyanoguanidino)dodecane (100 mg, 0.30 mmol) and 1,12-diaminododecane (60 mg, 0.30 mmol) were dissolved in DMSO (200 μL) followed by addition of aq. HCl (37%, 2.5 mL) and heated at 160° C. overnight. After cooling the resulting brown mixture to rt, the solvent was evaporated under high vacuum. The product was purified by preparative HPLC equipped with a C18-reverse phase column (H₂O/CH₃CN/formic acid 95:5:0.1 to 0:100:0.1) to afford a light brown solid. (24%)¹H-NMR (400 MHz, DMSO-d₆) δ: 8.41 (s, 2H, formate), 8.21 (brs, 4H, ex), 7.82-7.15 (brs, 16H, ex), 3.05 (q, J=6.3 Hz, 8H), 1.45 (m, 8H), 1.26 (brs, 32H) ppm. ¹³C-NMR (100.6 MHz, DMSO-d₆) δ: 167.7 (formate), 157.6, 41.1, 29.4 (2C), 29.1, 28.9, 26.5 ppm. HRMS (ESI+) m/z: calculated for C₂₈H₆₈N₁₂ [M+2NH₄+2H]⁴⁺ 143.1417, found 143.1417.

### Example 2: Inflammation

Here, the inventors investigate the molecular mechanisms underlying the regulation of metabolic and epigenetic plasticity of macrophages. They further evaluate the potential of reprogramming cell identity using a novel small molecule towards a less inflammatory signature.

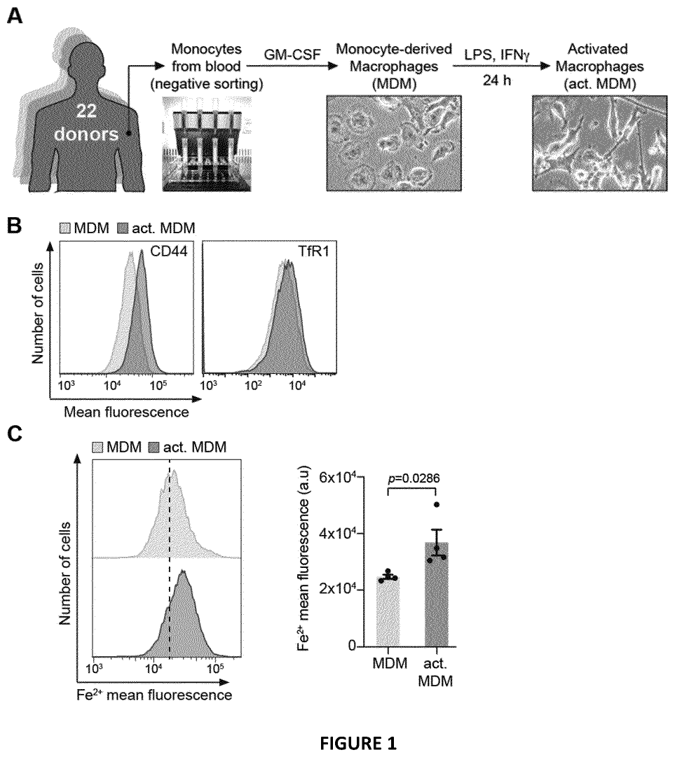

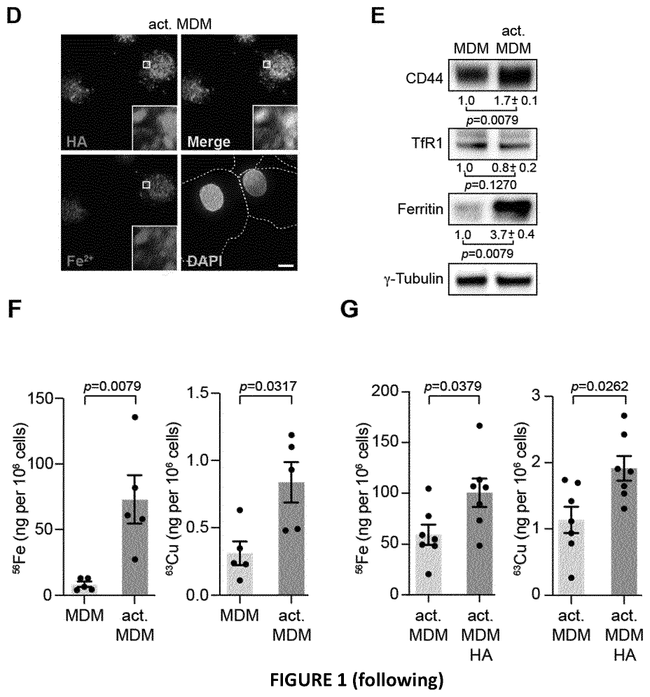

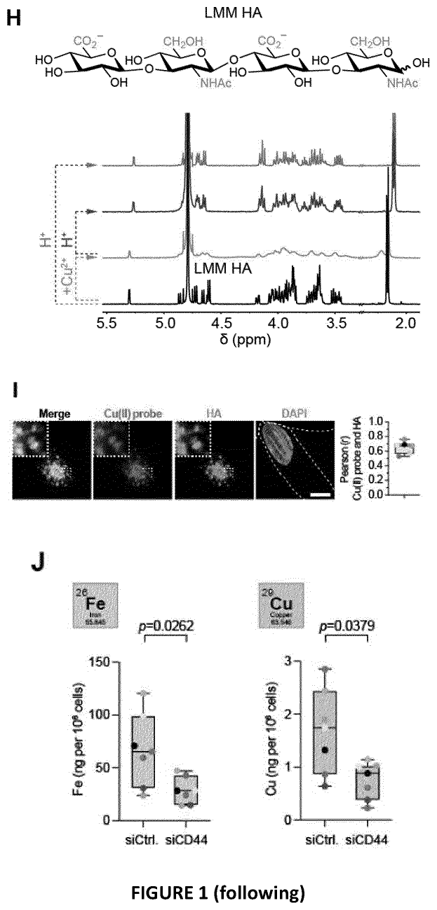

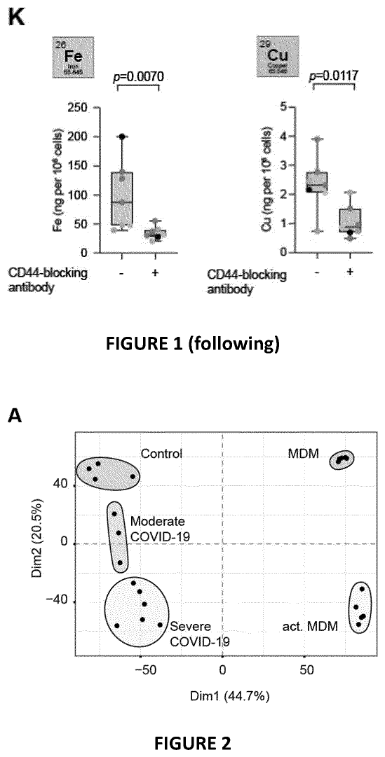

To study the role of CD44/HA-mediated metal uptake in inflammatory macrophages, the inventors isolated monocytes from human donors and differentiated them using granulocyte-macrophage colony-stimulating factor (GM-CSF) to produce monocyte-derived macrophages (MDM). They then activated MDM (act. MDM) using lipopolysaccharide (LPS) and interferon gamma (IFNγ) to generate inflammatory macrophages (FIG. 1A). In activated MDM, they observed increased levels of CD44 at the plasma membrane (FIG. 1B). In contrast, changes of transferrin receptor (TfR1) were marginal (FIG. 1B). Activated MDM exhibited enhanced iron endocytosis as defined by a fluorescent iron(II)-specific lysosomal probe (FIG. 1C), which co-localized with a fluorescently labeled HA (FIG. 1D). Ferritin levels increased together with CD44, indicating enhanced iron uptake in activated MDM (FIG. 1E). Accordingly, inductively coupled plasma mass spectrometry (ICP-MS) indicated that cellular levels iron and copper were higher in activated MDM (FIG. 1F). Consistent with this, supplementing cells with a high-molecular-mass (HMM) HA of a size of naturally occurring human HA (0.6-1 MDa), further increased levels of iron and copper in activated MDM (FIG. 1G). The inventors then studied the propensity of HA to form organometallic complexes with copper using a low-molecular-mass (LMM) HA, whose proton signals can be resolved by nuclear magnetic resonance spectroscopy. Adding copper(II) to LMM HA in water led to a line broadening of the proton signals of LMM HA (FIG. 1H). Acidifying the media to protonate the free carboxylate of HA and to disrupt copper binding restored the signals of unbound HA, showing that HA can dynamically interact with copper(II). Activated MDM exhibited enhanced copper endocytosis as defined by a fluorescent copper(II)-specific lysosomal probe, which co-localized with a fluorescently labeled HA (FIG. 11). Interestingly, downregulation of CD44 using a small interfering RNA (siRNA) (FIG. 1J) or using a CD44-blocking antibody (FIG. 1K), significantly reduced iron and copper uptake in macrophages. Taken together, these data indicate that CD44 and HA promote the uptake of iron and copper in activated MDM.

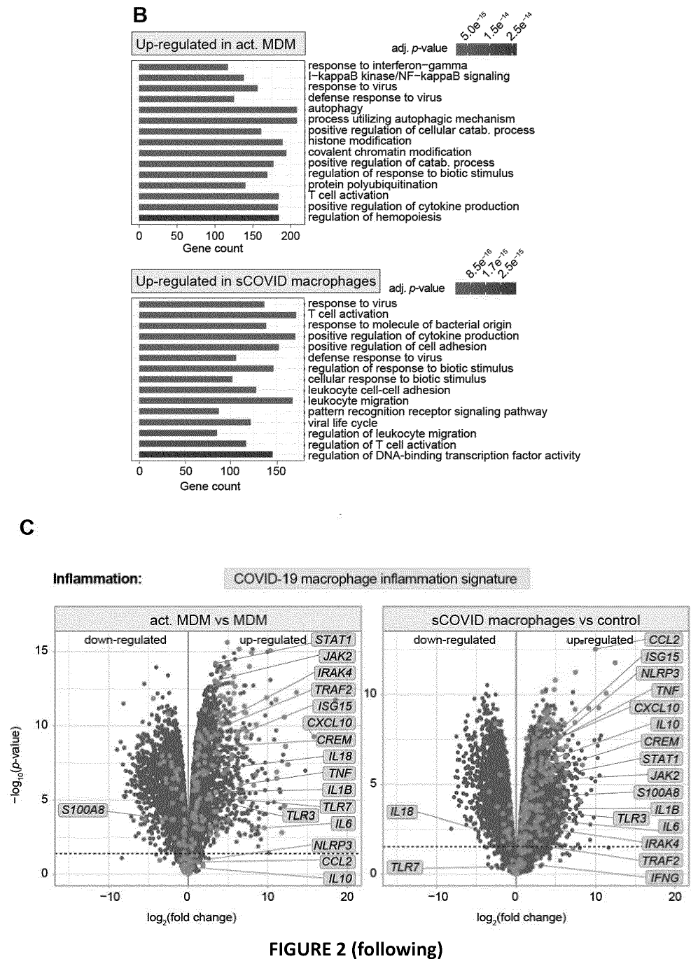

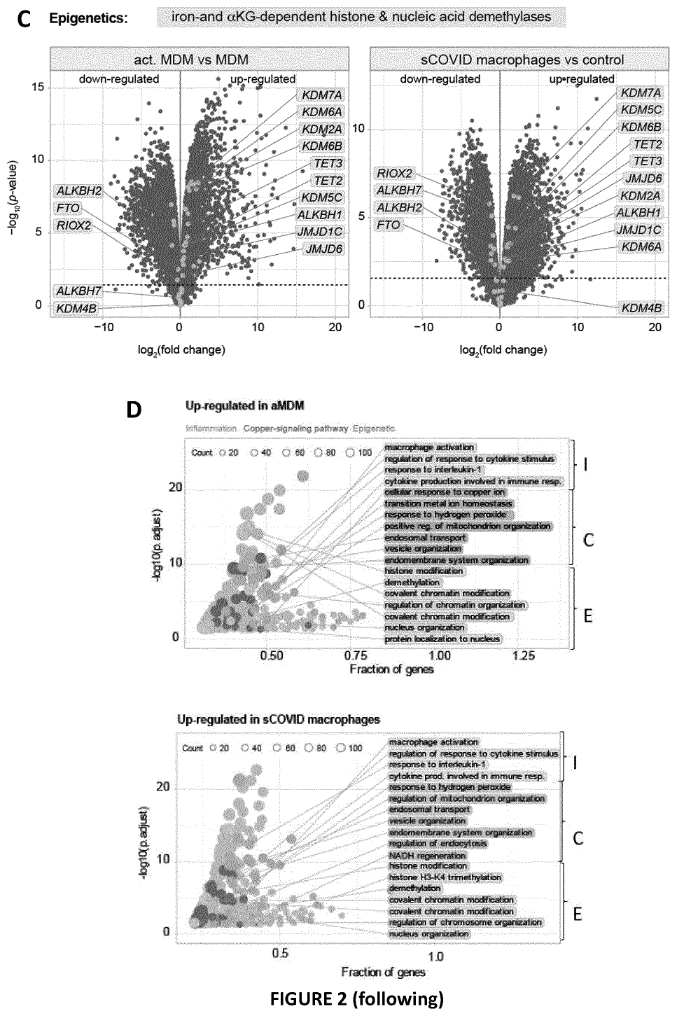

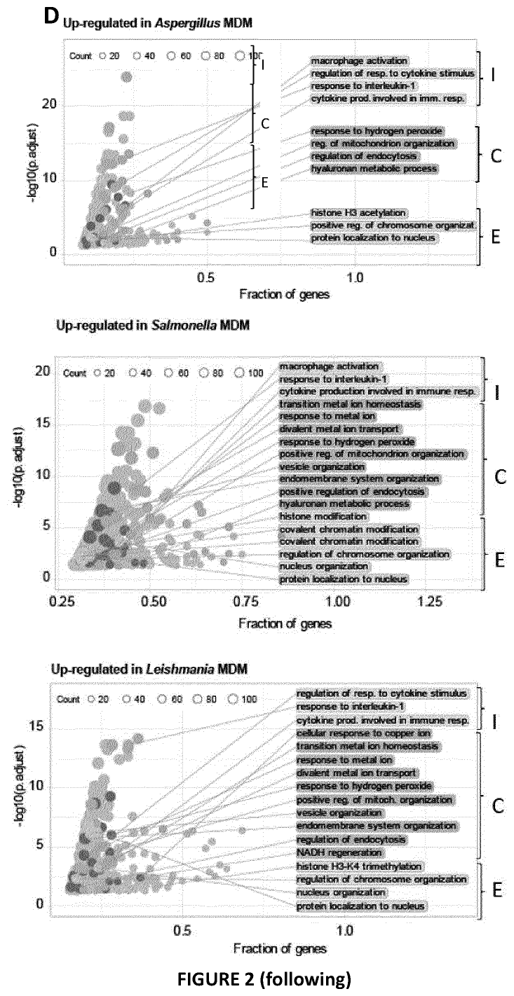

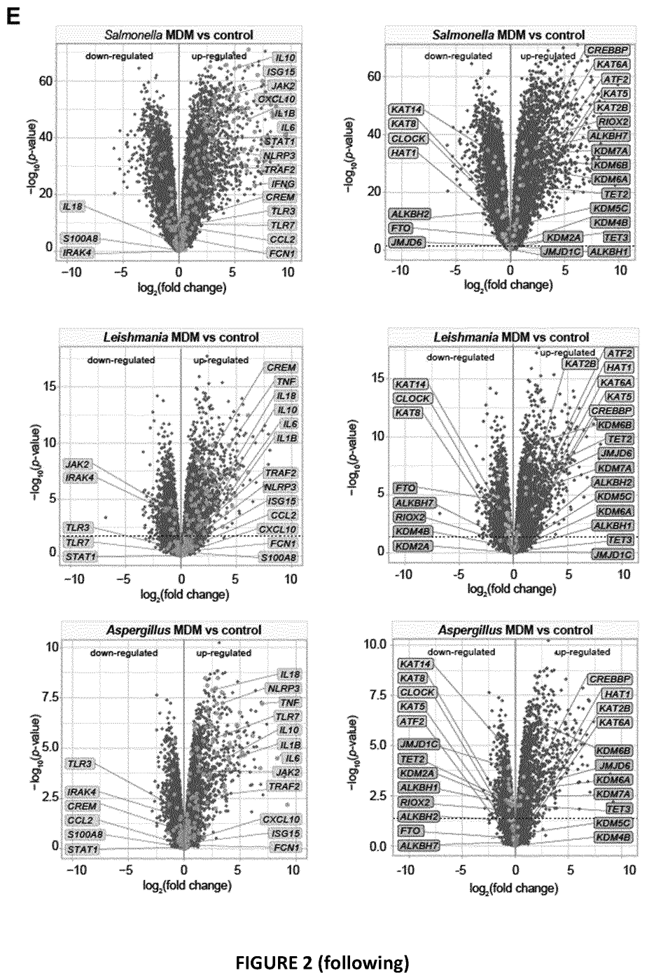

Next, the inventors performed RNA-seq to compare the gene expression signatures of activated MDM with that of macrophages obtained from BALF of severe COVID-19 (sCOVID) patients. These datasets revealed striking similarities between activated MDM and sCOVID macrophages compared to samples from moderate COVID-19 patients and controls (FIG. 2A). Gene ontology (GO) analyses pointed towards inflammatory genes being similarly up-regulated in activated MDM and sCOVID macrophages (FIG. 2B). For example, activated MDM and sCOVID macrophages exhibited up-regulated genes coding for inflammatory cytokines including IL-6, IL-1β and TNFα, for proteins involved in the JAK/STAT signaling pathway, for the inflammasome and for Toll-Like Receptors (TLRs) (FIG. 2C). Genes involved in chromatin and histone modifications were also up-regulated (FIG. 2B). Importantly, the expression of a subset of genes coding for iron- and alpha-ketoglutarate (αKG)-dependent demethylases that target active and repressive chromatin marks was also up-regulated (FIG. 2C). Consistent with iron acting as a rate-limiting regulator of epigenetic plasticity leading to inflammatory macrophages, activated MDM were characterized by changes in the status of histone marks, specific substrates of these demethylases. These data are in line with previous findings showing that the production of inflammatory macrophages involves epigenetic alterations. In activated MDM and sCOVID macrophage gene sets, sorting nexin 9 (SNX9), which regulates CD44 endocytosis was up-regulated. The gene coding for the iron storage protein ferritin heavy chain 1 (FTH1) was highly expressed. In addition, genes coding for metallothioneins (MT2A, MT1X), which are involved in copper homeostasis, were up-regulated. The inventors then compared the transcriptomics data on MDM and the transcriptomics data obtained from bronchoalveolar macrophages of patients infected with SARS-CoV-2 to transcriptomics data from human macrophages exposed in vitro to Salmonella typhimurium, Leishmania major or Aspergillus fumigatus. Interestingly, both GO-term analyses (FIG. 2D) and gene signatures (FIG. 2E) showed striking similarities between these datasets, confirming the inventors' mechanism in different inflammation settings. Taken together, these data support increase of cellular iron and copper in inflammatory macrophages. The implication of iron- and αKG-dependent demethylases, together with the established role of αKG in the regulation of macrophage plasticity, prompted us to investigate pathways involved in the production of αKG, including the Krebs cycle and glutamate metabolism. Interestingly, activated MDM and sCOVID macrophages exhibited changes of genes coding for metabolic enzymes, with an increase of glutamate metabolism. Together, these data advocate for a coordinated mechanism whereby macrophages enhance metal uptake to mediate epigenetic alterations required for the expression of inflammatory genes.

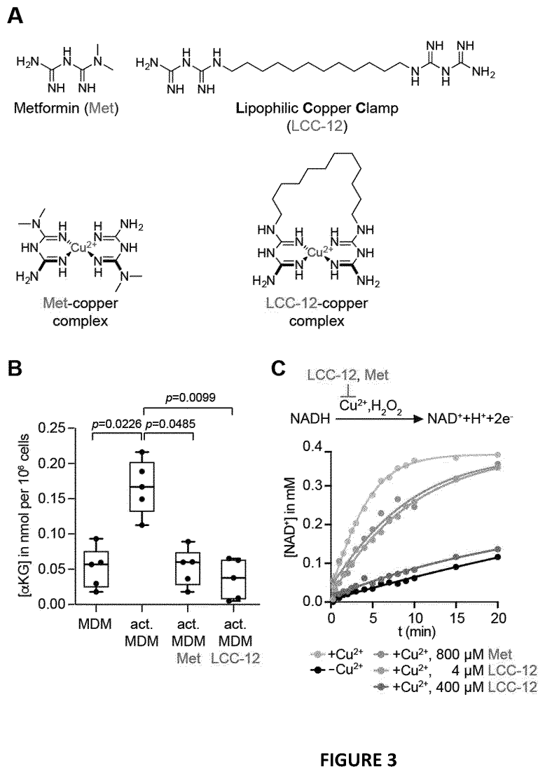

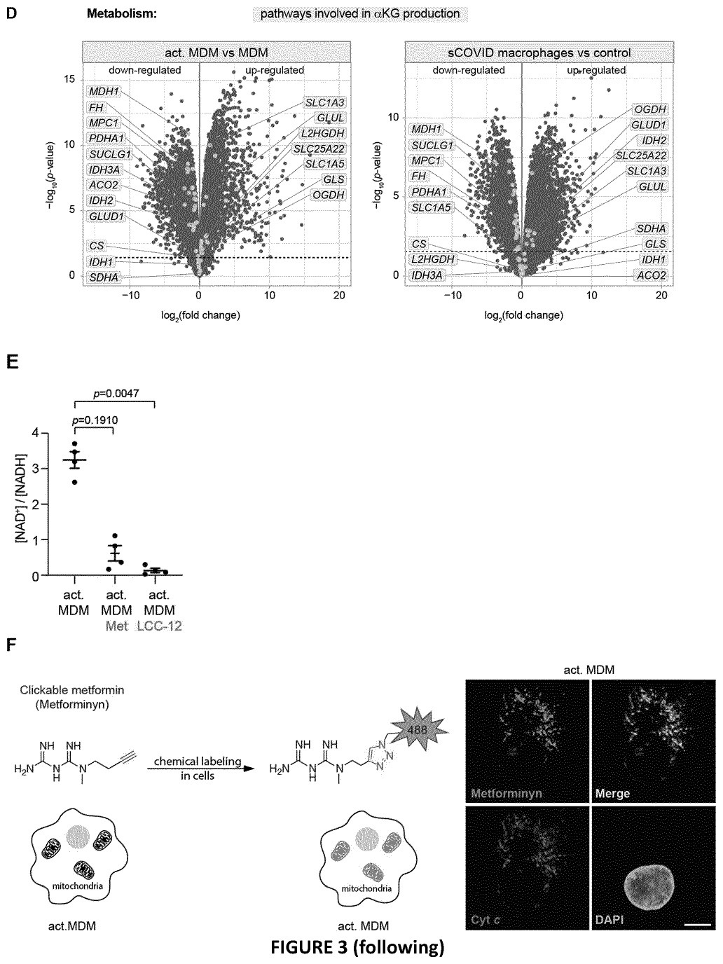

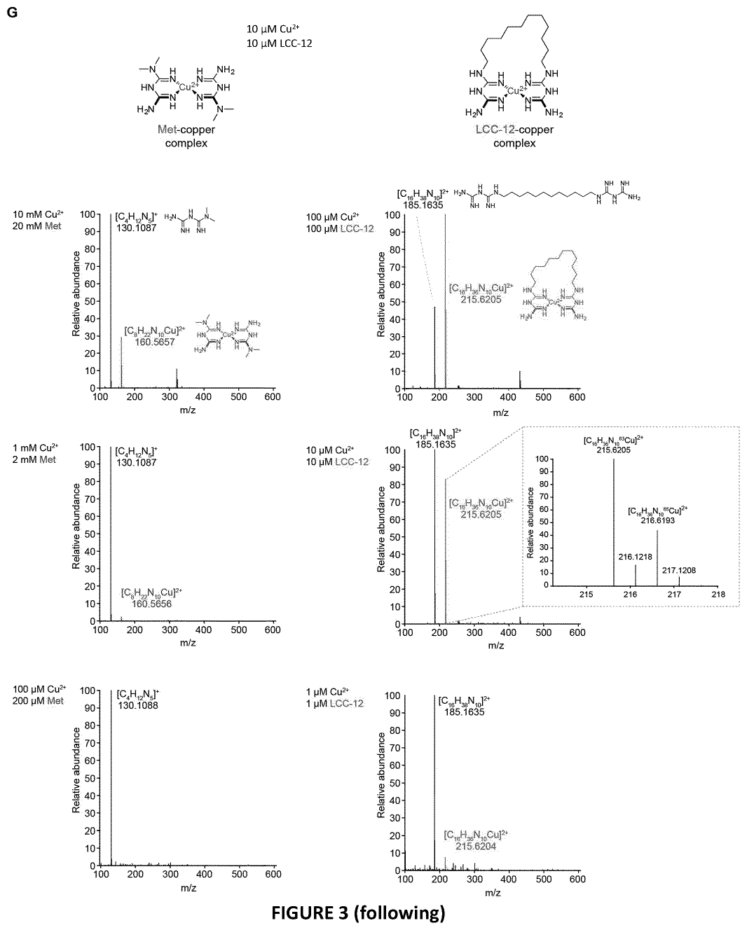

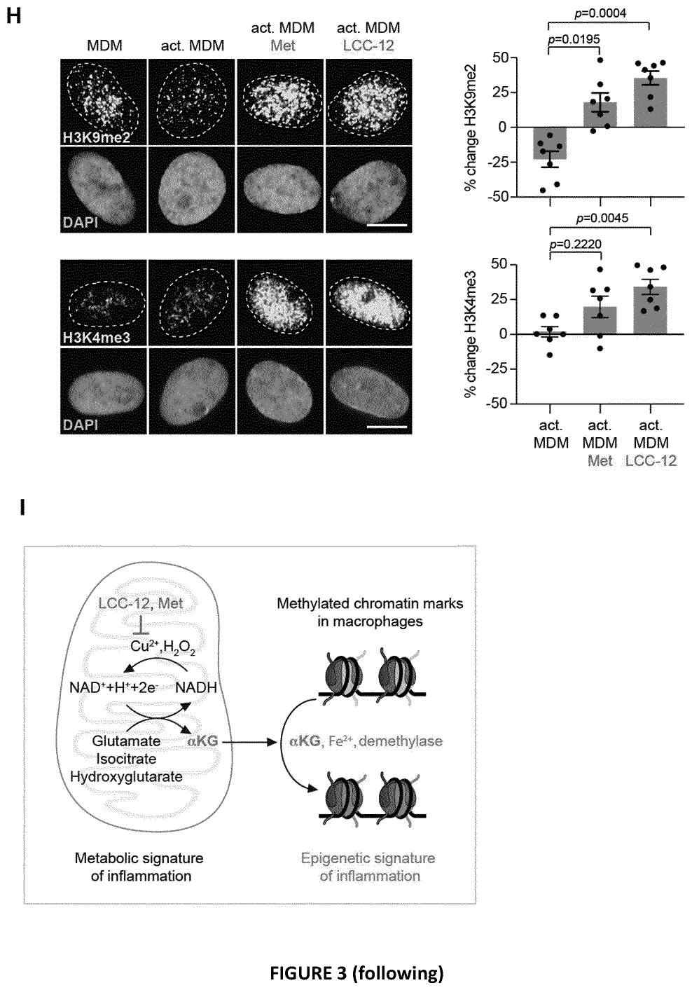

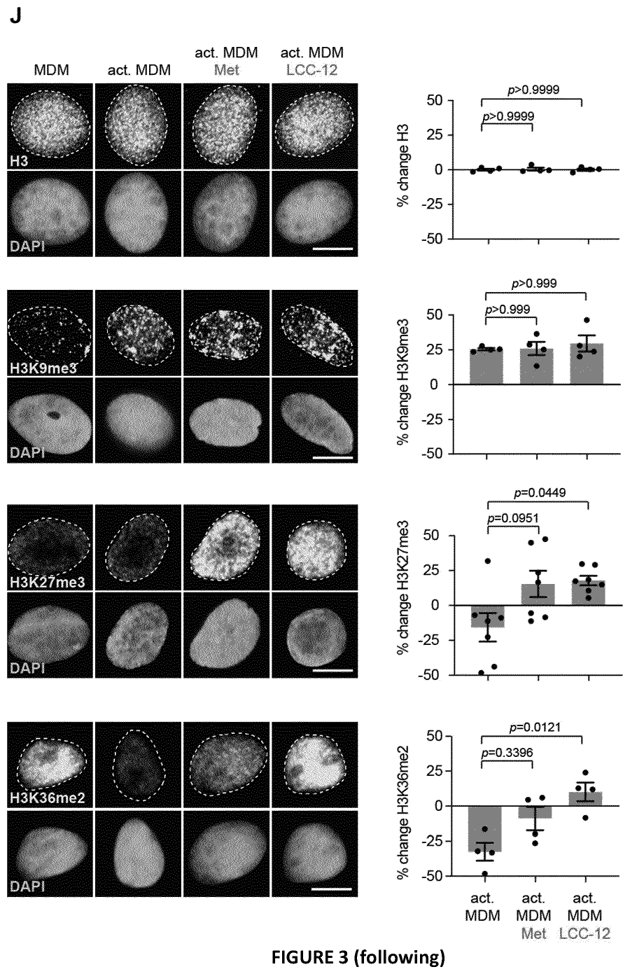

The clinically-approved biguanide metformin (Met) has been shown to reduce the production of αKG in cancer cells, although with a moderate potency. Interestingly, Met can form bimolecular organometallic complexes with copper. To alleviate the entropic cost inherent to the formation of a bimolecular complex, the inventors synthesized a dimeric small molecule, termed lipophilic copper clamp C₁₂ (LCC-12), where two biguanides were chemically tethered with a methylene-containing linker (FIG. 3A). Activated MDM were characterized by an increase of αKG, which was antagonized upon treatment with biguanides, LCC-12 being more potent than Met at a dose 1000-fold lower (FIG. 3B). This supports the notion that copper is a regulator of metabolic plasticity in macrophages. Copper(II) can catalyze the conversion of NADH into NAD⁺ in the presence of hydrogen peroxide, which can be found in mitochondria. Biguanides reduced the rate of NADH oxidation catalyzed by copper(II) with LCC-12 showing a more pronounced effect (FIG. 3C). Importantly, NAD⁺ is an enzymatic co-factor ubiquitously used in the production of αKG (FIG. 3D), and biguanides decreased the NAD⁺/NADH ratio in activated MDM (FIG. 3E). These data validate copper as a mechanistic target of biguanides and shed light on previously observed effects of Met on the activity of enzymes involving NAD⁺/NADH. Labeling a biologically active alkyne-containing analogue of Met in cells by click chemistry revealed a co-localization with cytochrome c, showing that biguanides predominantly target mitochondria in macrophages (FIG. 3F). LCC-12 formed a mono-adduct with copper(II) at low-micromolar concentrations, whereas substantially higher concentrations of Met were required to detect a bimolecular copper complex (FIG. 3G). These data further support mitochondrial copper as a target of biguanides. Remarkably, biguanides antagonized histone demethylation in activated MDM leading to a reprogramming of the epigenetic landscape (FIG. 3H and FIG. 3J). This is in line with the functional role of copper in the regulation of metabolic plasticity. Taken together, these data advocate for a mechanism whereby inflammatory macrophages upregulate copper uptake to replenish the pool of NAD⁺, which is required for the production of αKG and the epigenetic regulation of inflammatory genes by iron- and αKG-dependent demethylases (FIG. 3I).

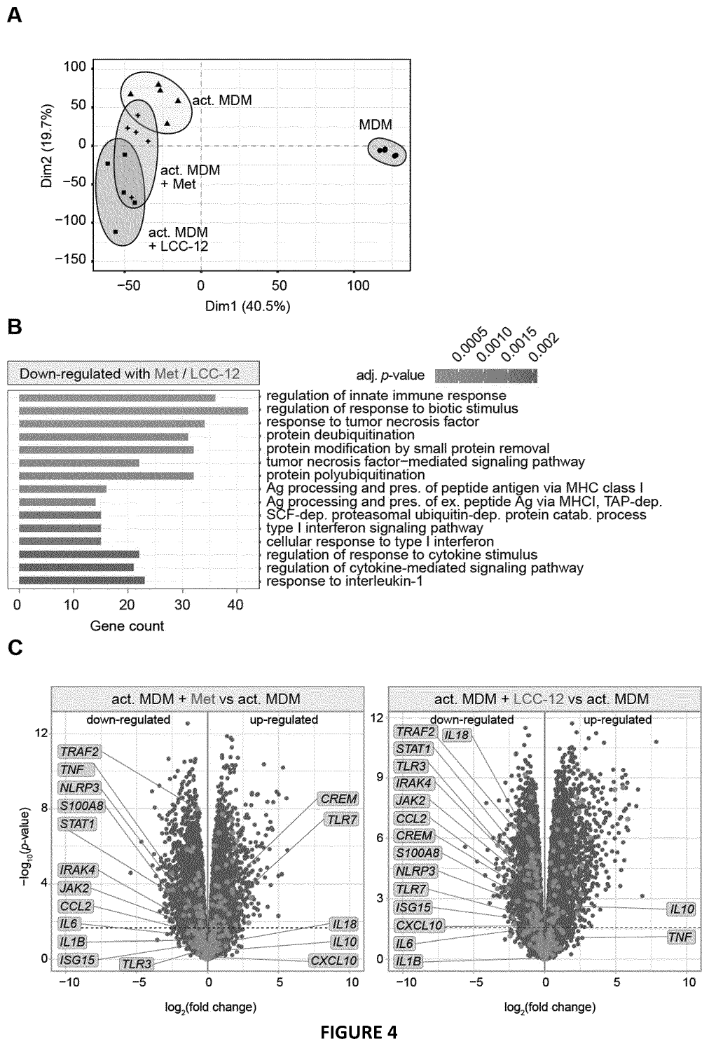

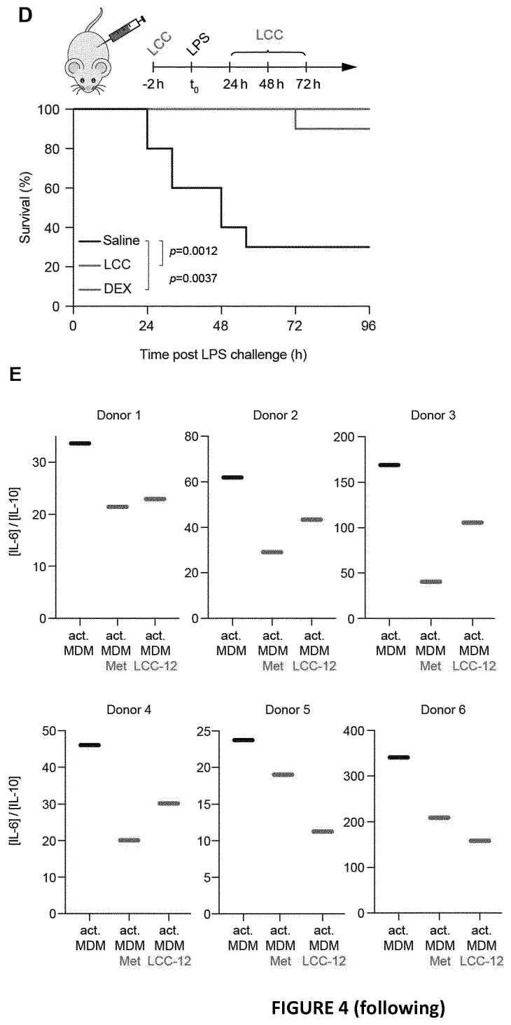

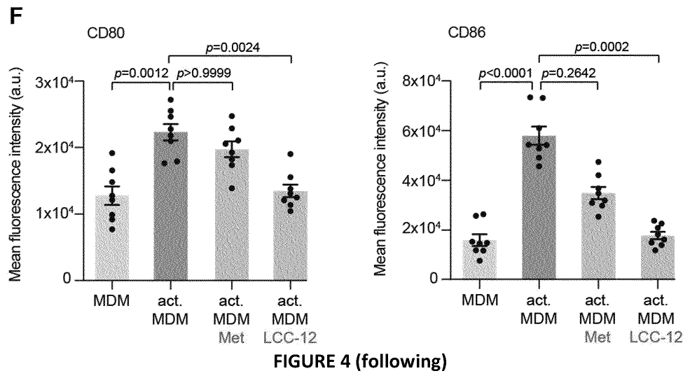

Next, the inventors evaluated the transcriptional effect and therapeutic potential of biguanides. LCC-12 treatment led to a different gene expression signature compared to that observed in activated MDM (FIG. 4A). This was defined by a reduced expression of a subset of inflammatory genes compared to activated MDM (FIGS. 4 B and C), characterizing a distinct macrophage state. For example, expression of IL-6, STAT1, JAK2, genes of the inflammasome, and genes coding for TLRs, all found to be up-regulated in activated MDM and sCovid macrophages, was reduced upon treatment with LCC-12. It is noteworthy that treatment with biguanides promoted expression of IL-10 in activated MDM, an anti-inflammatory cytokine previously shown to oppose metabolic reprogramming induced by inflammatory stimuli in macrophages. In particular, the ratio of IL-6 to IL-10 cytokines was reduced upon treatment of activated MDM with biguanides (FIG. 4E). Lower ratios of IL-6 to IL-10 correlate with moderate forms of COVID-19, supporting the anti-inflammatory effect of these small molecules. In support of biguanides treatment leading to a distinct state of macrophage, LCC-12 interfered with the production of inflammatory macrophages according to CD80 and CD86 cell surface markers (FIG. 4F). LCC-12 increased the survival of mice challenged with LPS to a similar extent than dexamethasone, an anti-inflammatory corticosteroid known to improve the condition of severe COVID-19 patients (FIG. 4D).

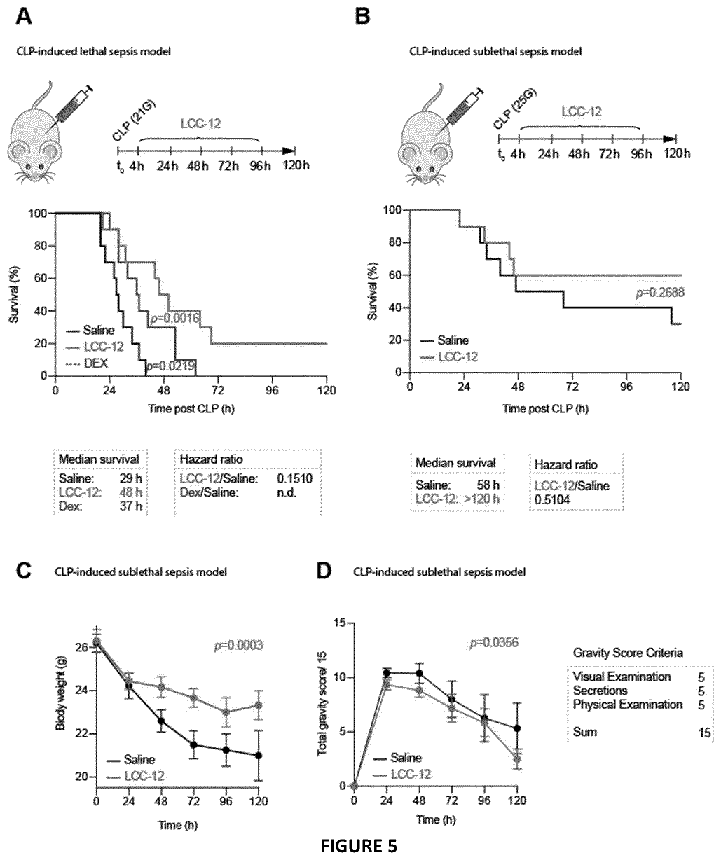

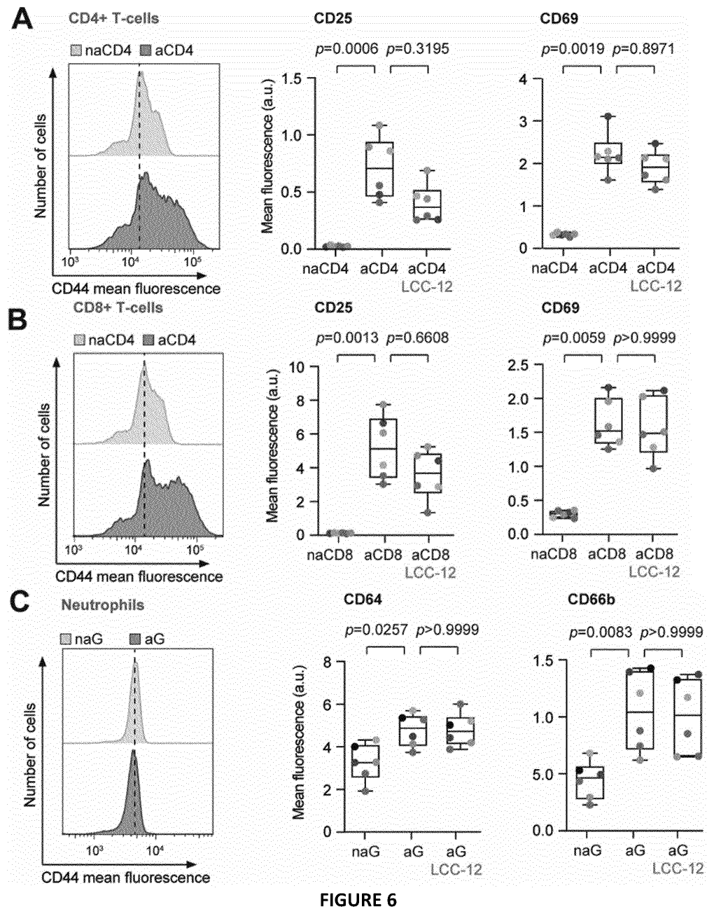

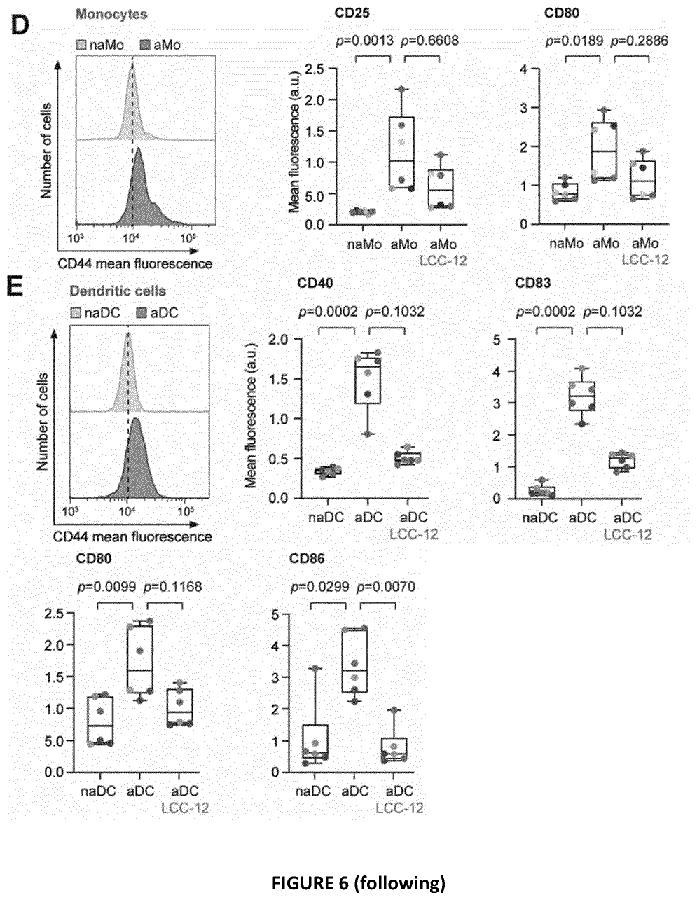

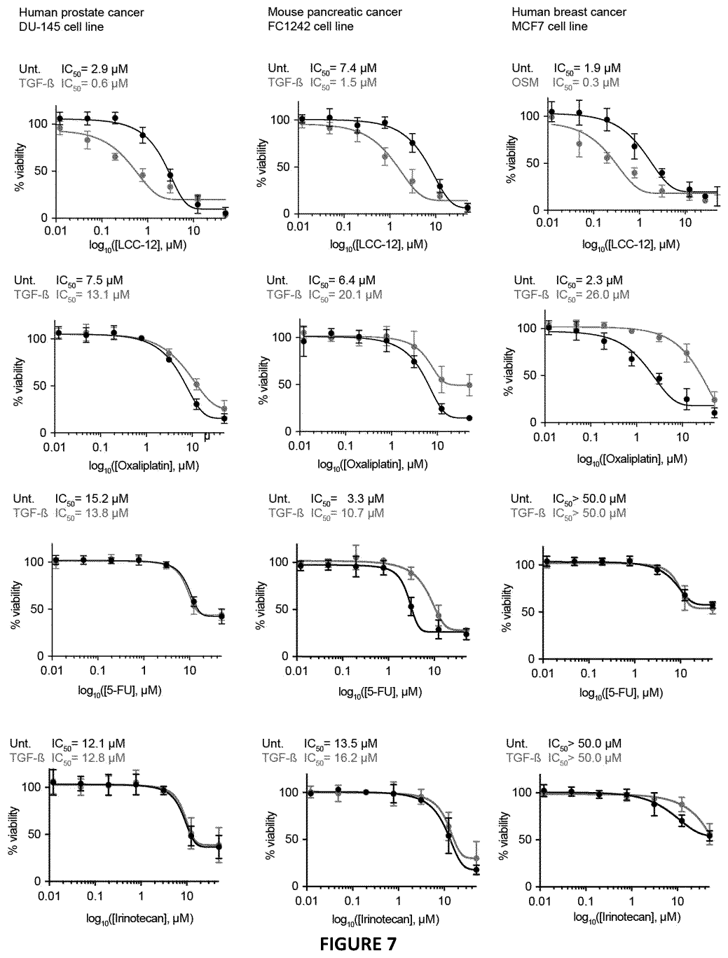

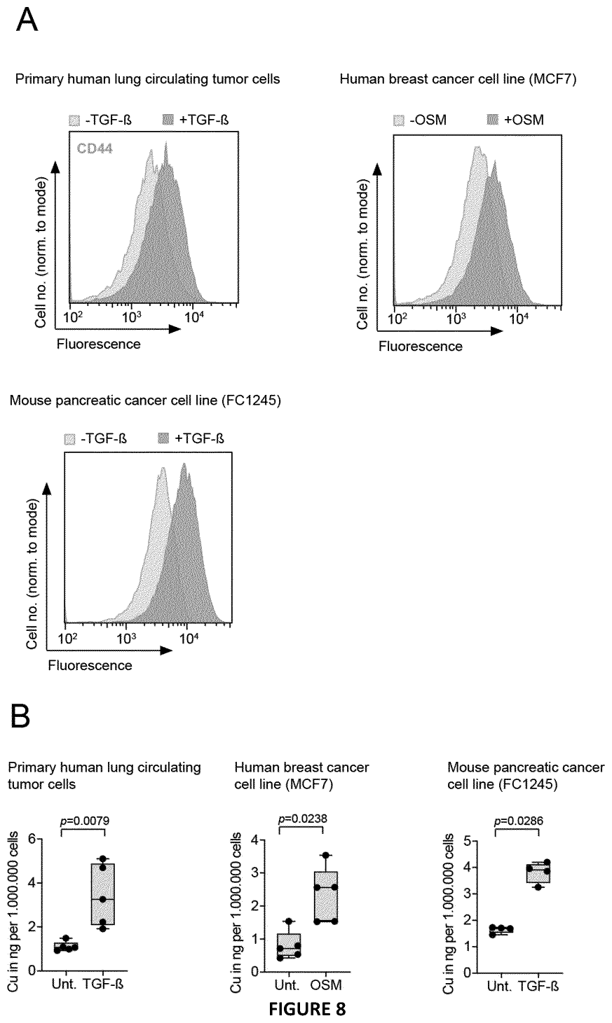

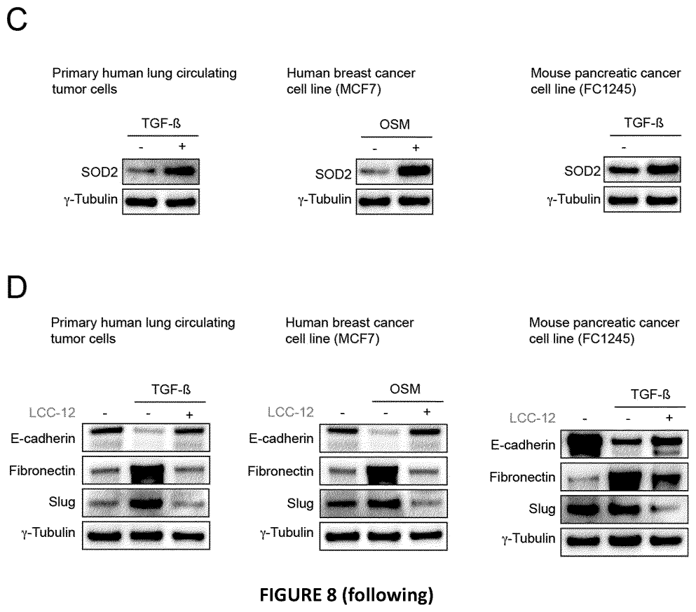

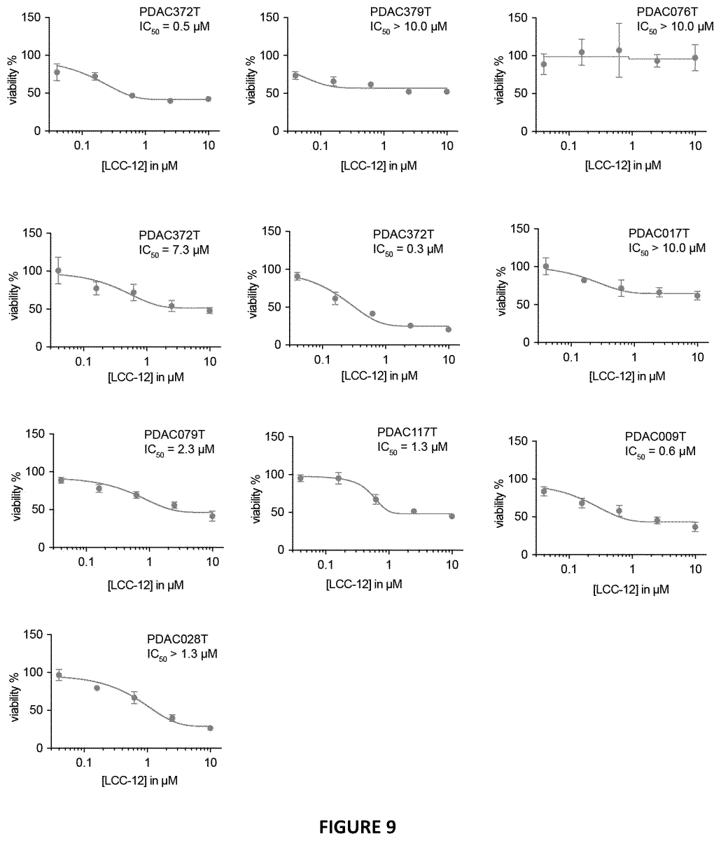

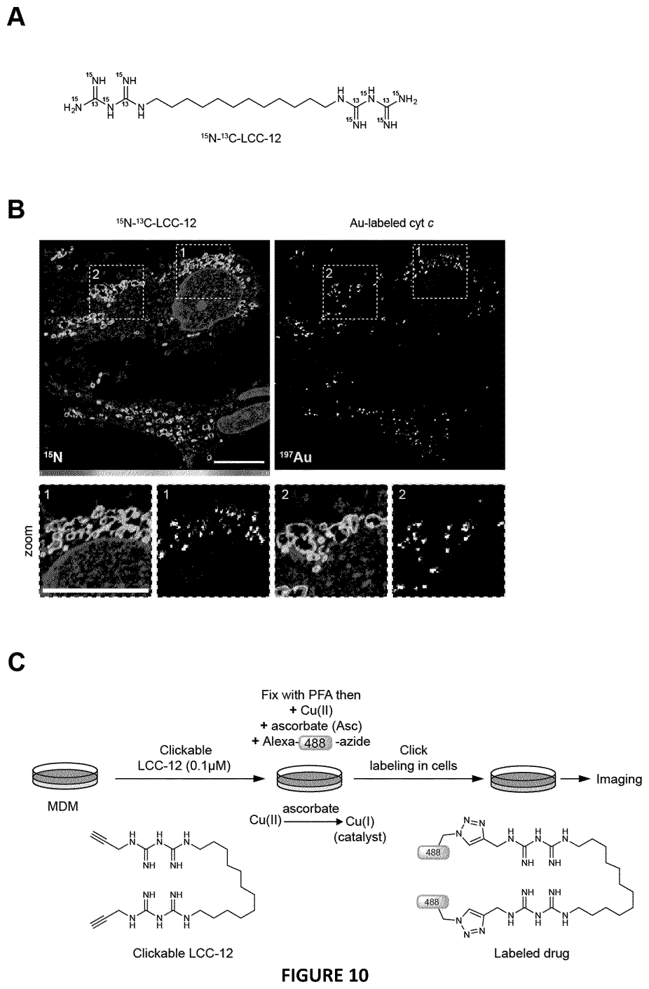

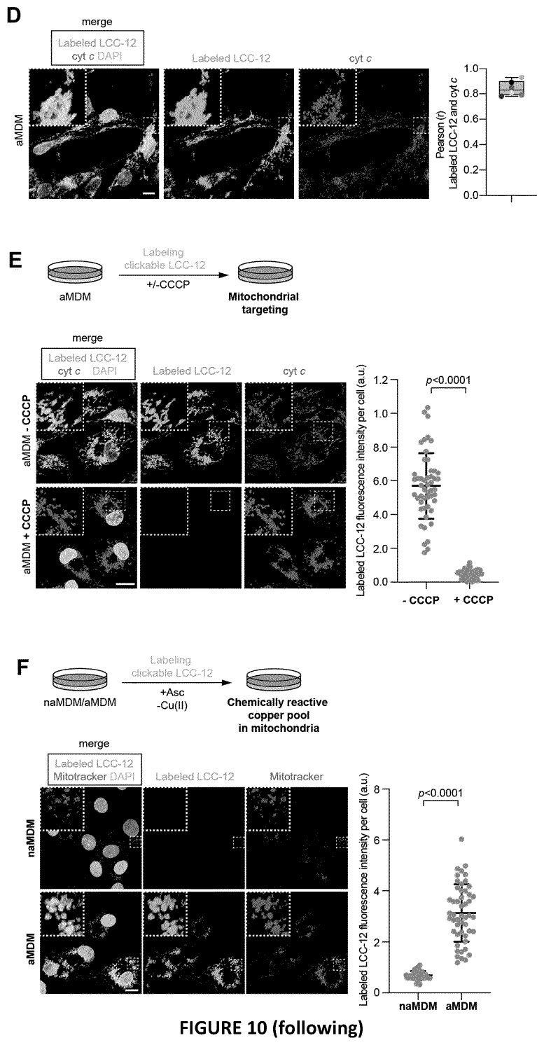

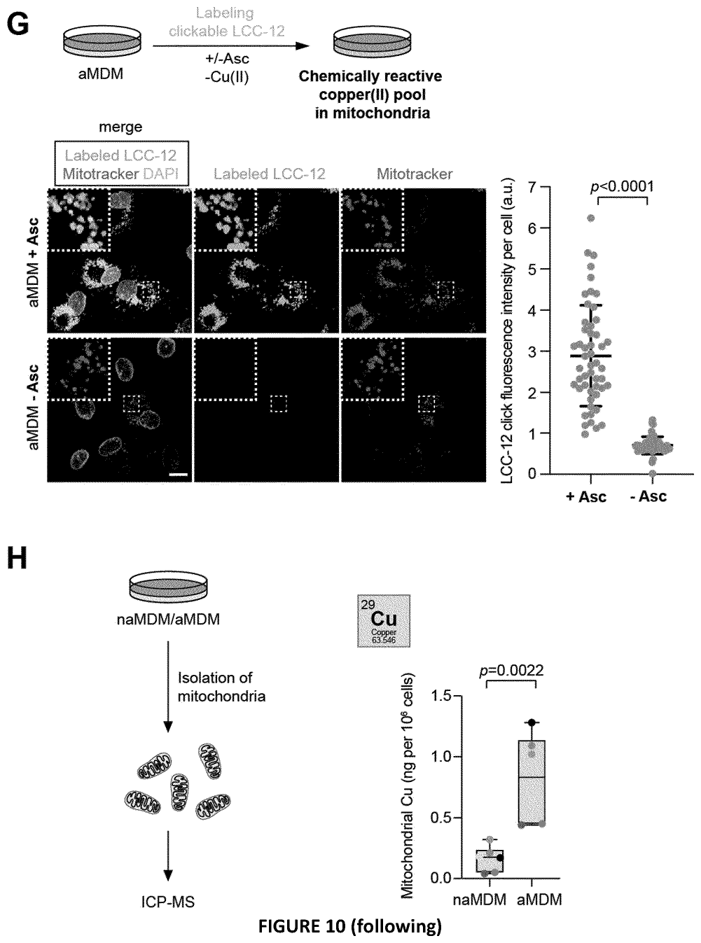

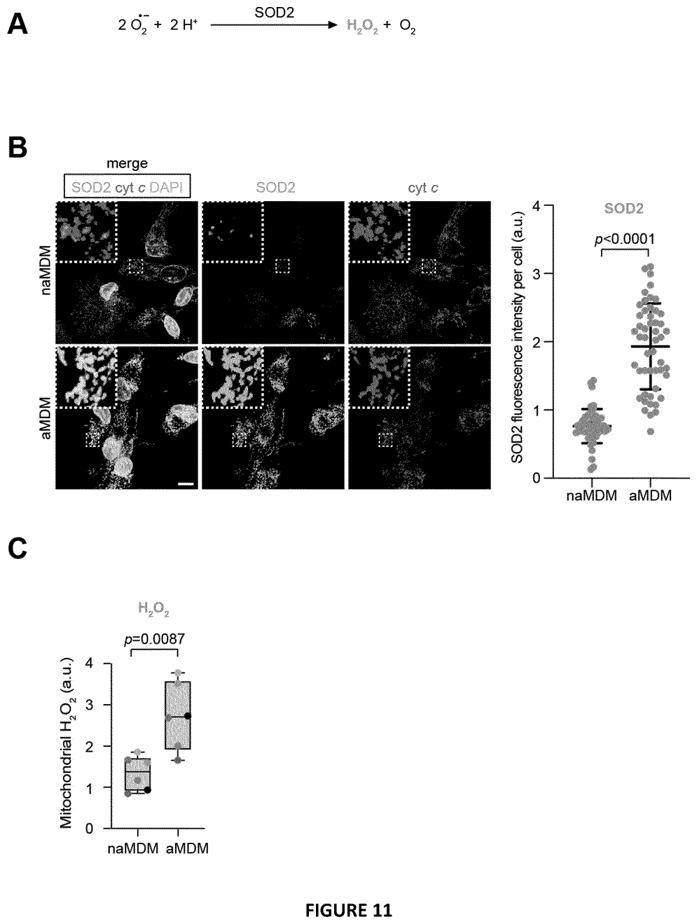

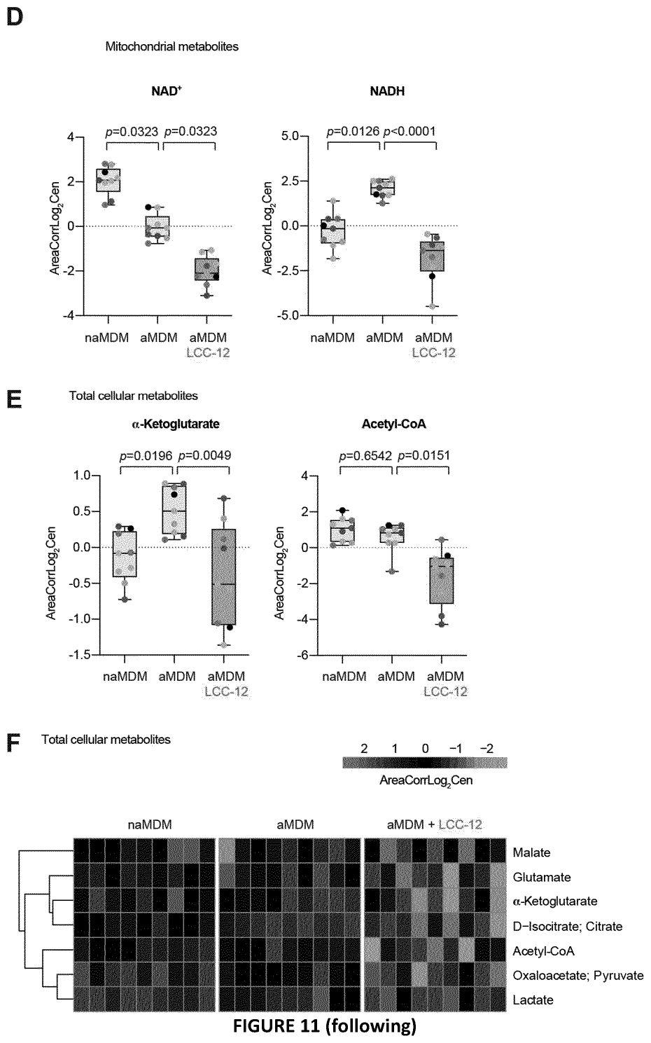

Finally, the inventors evaluated the effect of LCC-12 at 0.3 mg/kg/day, a dose 10 times lower than the maximum tolerated dose (MTD), in another model of sepsis, namely cecal ligation and puncture (CLP). This model is representative of the pathophysiology of subacute polymicrobial abdominal sepsis occurring in humans. LCC-12 delayed death and increased survival rate (FIG. 5A). A similar trend was observed in another model of CLP-induced sublethal sepsis where LCC-12 reduced body weight loss and improved the overall gravity score of symptoms (FIG. 5B-D). It is noteworthy that LCC-12 considerably attenuated the symptoms of sepsis compared to dexamethasone throughout the treatments. Taken together, these data illustrate the therapeutic potential of LCC-12.

In conclusion, two of the d-block metals are required for the generation of inflammatory macrophages and blocking the production of αKG and acetyl-CoA through mitochondrial copper targeting correlates with therapeutic benefits. This is further supported by the observed improved condition of COVID-19 patients undergoing treatment with Met. Notably, glucose metabolism, which can lead to αKG production (FIG. 3D), is dysregulated in patients with obesity and diabetes, two conditions that increase risks of morbidity with COVID-19. This work illuminates the central role of copper and iron as regulators of metabolic and epigenetic plasticity, providing the means to develop new therapeutics for the clinical management of inflammatory diseases.

Materials and Methods

Antibodies. (WB=Western blot, FC=Flow cytometry, FM=Fluorescence microscopy). CD44 (Abcam, ab189524, WB), CD44-Alexa-Fluor-647 (Novus Biologicals, NB500-481AF647, FC), CD80-AlexaFluor700 (Becton, Dickinson and Company (BD), 561133, FC), CD86-PE/Cy7 (BD, 561128, FC), Cytochrome c (cyt c, Cell Signaling, 12963S, FM), Ferritin (Abcam, ab75973, WB), H3 (Cell Signaling, 9715S, FM), H3K4me3 (Diagenode, C15410003-50, FM), H3K9me2 (Cell Signaling, 4658S, FM), H3K9me3 (Cell Signaling, 13969S, FM), H3K27me3 (Cell Signaling, 9733S, FM), H3K36me2 (Abcam, ab9049, FM), TfR1-APC/Alexa750 (Beckman Coulter, A89313, FC), Transferrin receptor 1 (TfR1, Life Technologies, 13-6800, WB), γ-Tubulin (Sigma-Aldrich, T5326, WB).

Cell culture. Peripheral blood samples were collected from distinct healthy donors (Etablissement Français du Sang). Pan monocytes were isolated by negative magnetic sorting using microbeads according to the manufacturer's instructions (Miltenyi Biotec, 130-096-537), and cultured in RPMI 1640 supplemented with glutamine (Thermo Fisher Scientific, 61870010), 10% fetal bovine serum and exposed to granulocyte-macrophage colony-stimulating factor (GM-CSF, Miltenyi Biotec, 130-093-866, 100 ng/mL) to induce differentiation into macrophages (MDM). At day 5 of differentiation, MDM were treated with lipopolysaccharides (LPS, InvivoGen, tlrl-3pelps, 100 ng/mL, 24 h) and interferon-γ (IFNγ, Miltenyi Biotec, 130-096-484, 20 ng/mL, 24 h) to generate activated MDM (act. MDM) and were co-treated with metformin (Met, 1,1-dimethylbiguanid hydrochloride, Alfa Aesar, J63361, 10 mM, 24 h) or LCC-12 (in-house, 10 μM, 24 h) as indicated.

Flow cytometry. Cells were washed with ice-cold 1×PBS, incubated with Fc block (Human TruStain FcX, Biolegend, 422302, 1/20) for 15 min, incubated with antibodies for 20 min at 4° C. and washed before analysis using a BD LSRFortessa X-20. Macrophages were analyzed with an antibody panel consisting of antibodies against the following cell surface proteins: CD44, CD80, CD86 and TfR1. The data were analyzed with FlowJo software v. 10.0.00003. Flow cytometry analysis of lysosomal iron content: Lysosomal iron was monitored by incubating cells at 37° C. with 5% CO₂ in medium containing RhoNox-M (in-house, 1 μM, 1 h), before flow analysis of fluorescence intensity.

Fluorescence microscopy. Isolated monocytes were plated on cover slips, differentiated and activated as described in cell culture. For fluorescent detection of HA, Fe²⁺ and Cu²⁺, live cells were treated with HA-FITC (800 kDa, Carbosynth, YH45321, 0.1 mg/mL, ˜125 μM) and RhoNox-M (Niwa et al., 2014, Org. Biomol. Chem. 12, 6590-6597) (in-house, 1 μM) or Lys-Cu (Ren et al., 2015, J. Mater Chem. B 3, 6746-6752) (in-house, 20 μM) for 1 h before fixation. Cells were washed three times with 1×PBS, fixed with 2% paraformaldehyde in 1×PBS for 12 min and then washed three times with 1×PBS. After fixation, cells were permeabilized with 0.1% Triton X-100 in 1×PBS for 5 min and washed three times with 1×PBS. Subsequently, cells were blocked in 2% BSA, 0.2% Tween-20/1×PBS (blocking buffer) for 20 min at room temperature. Cells were incubated with the relevant antibody in blocking buffer for 1 h at room temperature, washed three times with 1×PBS and were incubated with secondary antibodies for 1 h. Finally, cover slips were washed three times with 1×PBS and mounted using VECTASHIELD (Vector Laboratories) containing DAPI. Fluorescence images were acquired using a Deltavision real-time microscope (Applied Precision). 40×/1.4NA, 60×/1.4NA and 100×/1.4NA objectives were used for acquisitions and all images were acquired as z-stacks. Images were deconvoluted with SoftWorx (Ratio conservative—15 iterations, Applied Precision) and processed with ImageJ. Histone quantification was performed delineating the nuclei using DAPI fluorescence.

Bright-field and digital photographs. Bright field images were acquired using a CKX41 microscope (Olympus) and cellSens Entry imaging software (Olympus). Digital images were taken with an iPhone 11 Pro (Apple).

Click labeling. Activated MDM on coverslips were treated with metforminyn (in-house, 10 μM, 3 h) fixed and permeabilized as indicated in fluorescence microscopy. The click reaction cocktail was prepared from a Click-iT EdU Imaging kit (C10337, Life Technologies) according to the manufacturer's protocol. Briefly, mixing 430 μL of 1× Click-iT reaction buffer with 20 μL of CuSO₄ solution, 1.2 μL Alexa Fluor azide, 50 μL reaction buffer additive (sodium ascorbate) to reach a final volume of ˜500 μL. Cover-slips were incubated with the click reaction cocktail in the dark at room temperature for 30 min, then washed three times with 1×PBS. Immunofluorescence was then performed as described in fluorescence microscopy.

Western Blotting. Cells were treated as indicated and then washed with 1×PBS. Proteins were solubilized in 2× Laemmli buffer containing benzonase (VWR, 70664-3, 1:100), extracts were incubated at 37° C. for 1 h, and quantified using a NanoDrop 2000 spectrophotometer (ThermoFisher Scientific). Protein lysates were resolved by SDS-PAGE electrophoresis (Invitrogen sure-lock system and Nu-PAGE 4-12% Bis-Tris precast gels) and transferred onto nitrocellulose (Amersham Protran 0.45 μm) membranes using a Trans-Blot SD semi-dry electrophoretic transfer cell (Bio-rad). Membranes were blocked with 5% non-fat skimmed milk powder in 0.1% Tween-20/1×PBS for 1 h. Blots were then probed with the relevant primary antibodies in 5% BSA, 0.1% Tween-20/1×PBS at 4° C. overnight with gentle motion. Membranes were washed with 0.1% Tween-20/1×PBS three times and incubated with horseradish peroxidase conjugated secondary antibodies (Jackson Laboratories) in 5% non-fat skimmed milk powder, 0.1% Tween-20/1×PBS for 1 h at room temperature and washed three times with 0.1% Tween-20/1×PBS. Antigens were detected using the SuperSignal West Pico PLUS chemiluminescent detection kits (ThermoFisher Scientific, 34580 and 34096). Signals were recorded using a Fusion Solo S Imaging System (Vilber) and quantified as indicated using ImageJ.

Inductively coupled plasma mass spectrometry (ICP-MS). HA (Carbosynth, FH45321, 600-1000 kDa, 1 mg/mL) was added together with LPS and IFNγ and cells were treated for 24 h. Glass vials equipped with Teflon septa were cleaned with nitric acid 65% (VWR, Suprapur, 1.00441.0250), washed with ultrapure water (Sigma-Aldrich, 1012620500) and dried. Cells were harvested followed by two washes with 1×PBS. Cells were then counted using an automated cell counter (Entek) and transferred in 200 μL 1×PBS to the cleaned glass vials, and samples were lyophilized using a freeze dryer (CHRIST, 22080). Samples were subsequently mixed with nitric acid 65% overnight and heated at 80° C. for 2 h. Samples were diluted with ultrapure water to a final concentration of 0.475 N nitric acid and transferred to metal-free centrifuge vials (VWR, 89049-172) for subsequent ICP-MS analysis. Amounts of ⁵⁶Fe and ⁶³Cu were measured using an Agilent 7900 ICP-QMS in low-resolution mode. Sample introduction was achieved with a micro-nebulizer (MicroMist, 0.2 mL/min) through a Scott spray chamber. Isotopes were measured using a collision-reaction interface with helium gas (5 mL/min) to remove polyatomic interferences. Scandium and indium internal standards were injected after inline mixing with the samples to control the absence of signal drift and matrix effects. A mix of certified standards was measured at concentrations spanning those of the samples to convert count measurements to concentrations in the solution. Uncertainties on sample concentrations were calculated using algebraic propagation of ICP-MS blank and sample counts uncertainties. Values were normalized against cell number.

siRNA Transfection and CD44 blocking antibody. Human primary monocytes were transfected with Human Monocyte Nucleofector kit (Lonza, VPA-1007) according to the manufacturer's instructions. Briefly, 5×10⁶ monocytes were resuspended into 100 μL of nucleofector solution with 200 pmol of ON-TARGETplus CD44 SMARTpool siRNA (Horizon Discovery, L-009999-00-0050) or negative control siRNA (Qiagen, 1027310) before nucleofection with Nucleofector II (Lonza). Cells were then immediately removed and incubated overnight with 5 mL of prewarmed complete RPMI medium (Thermo Fisher Scientific). The following day, CSF-1 was added to the medium. Cells were then treated with anti-human CD44 therapeutic antibody (RG7356, Creative Biolabs, TAB-128CL, 10 μg/mL, 24 h) as indicated.

NMR spectroscopy of HA:copper(II) complex. ¹H-NMR spectra were recorded on a 500 MHz Bruker spectrometer at 310 K, and chemical shifts δ are expressed in ppm using the residual non-deuterated solvent signal as internal standard. Portions of 0.25 mol equiv. of a solution of CuCl₂ in D₂O (8.6 mg in 599 μL D₂O) were added to a 2 mM solution of low-molecular-mass HA(LMM Hyal, TCI Chemicals, H1284) in D₂O (1 mg HA in 600 μL D₂O) up to 1 mol equiv. into an NMR tube. Then, a drop of trifluoroacetic acid (TFA, Alfa Aesar, A12198) was added. In a separate NMR tube, a drop of TFA was added to a 2 mM solution of LMM-HA in D₂O.

RNA-seq. RNAs were extracted using the RNeasy mini kit (QIAGEN, 74104). RNA sequencing libraries were prepared from 1 μg total RNA using the Illumina TruSeq Stranded mRNA library preparation kit (Illumina, 20020594), which allows strand-specific sequencing. A first step of polyA selection using magnetic beads was performed to allow sequencing of polyadenylated transcripts. After fragmentation, cDNA synthesis was performed and resulting fragments were used for dA-tailing followed by ligation of TruSeq indexed adapters (Illumina, 20020492). Subsequently, polymerase chain reaction amplification was performed to generate the final barcoded cDNA libraries. Sequencing was carried out on a NovaSeq 6000 instrument from Illumina based on a 2×100 cycle mode (paired-end reads, 100 bases). Raw sequencing reads were first checked for quality with Fastqc (0.11.8) and trimmed for adapter sequences with the trimGalore (0.6.2) software. Trimmed reads were then aligned on the human hg38 reference genome using the STAR mapper (2.6.1b), up to the generation of a raw count table per gene (GENCODE annotation v29). The bioinformatics pipelines used for these tasks are available online (rawqc v2.1.0: https://github.com/bioinfo-pf-curie/raw-qc, RNA-seq v3.1.4: https://github.com/bioinfo-pf-curie/RNA-seq). The downstream analysis was then restricted to protein-coding genes. Data from (Liao et al., 2020, Nat. Med. 26, 842-844) were converted into bulk by keeping cells annotated as macrophages and then summing the counts for each sample. Counts data from (Pai et al., 2016, PLoS Genet. 12, e1006338) were downloaded from GEO under accession number GSE73502. Raw data from (Fernandes et al., 2016, 7, e00027-16; Gongalves et al., 2020, Nat. Commun. 11, 2282) were downloaded from the NCBI Short Read Archive under records PRJNA528433 and PRJNA290995 and processed as described above. Counts were normalized using TMM normalization from edgeR (v 3.30.3) (Robinson et al., 2010, Bioinformatics 26, 139-140). Differential expression was assessed with the limma voom framework (v 3.44.3) (Ritchie et al., 2015, Nucleic Acids Res. 43, e47). The intra-donor correlation was controlled by using the duplicateCorrelation from limma. Genes with an adjusted p-value<0.05 were called significant. Enrichment analysis from differentially expressed genes has been performed using the enrichGO function from clusterProfilter package v3.16.1.

α-Ketoglutarate (αKG) measurements. αKG was quantified using a fluorometric assay (Abcam, ab83431) according to the manufacturer's protocol. At least 2×10⁶ cells were treated as indicated and harvested per condition. Floating cells were harvested and adherent cells were washed with 1×PBS. Adherent cells were incubated with 1×PBS with 10 mM EDTA and then scraped and pooled together with the harvested floating cells. Cells were subsequently washed with ice-cold 1×PBS and counted. Then, cells were re-suspended in ice-cold αKG buffer (kit component). Cells were centrifuged at 25000× g for 5 min at 4° C. and the supernatant was transferred to clean tubes. Ice-cold perchloric acid (Sigma-Aldrich, 311421-50ML) was added to a final concentration of 1 M and the solution was incubated on ice for 5 min. Cells were centrifuged at 13000× g for 2 min and the supernatant was transferred to a clean tube. Then, ⅓ vol. of a 2 M solution of KOH was added and the pH adjusted to 7.4 using a 0.1 M aq. solution of KOH. Samples were centrifuged at 13000× g for 15 min and the supernatants collected. αKG levels were measured using a standard curve and a control to subtract pyruvate background levels. Fluorescence intensities (ex. 535 nm; em. 590 nm) were recorded using a Perkin Elmer Wallac 1420 Victor2 Microplate Reader, and data were normalized against cell number. Values were derived from the standard curve for each experiment.

Synthesis. Products were purified on a preparative HPLC Quaternary Gradient 2545 equipped with a Photodiode Array detector (Waters) fitted with a reverse phase column (XBridge Prep C18 5 μm OBD 30×150 mm). Spectra were run in DMSO-d₆ or Methanol-d₆ at 298 K unless stated otherwise. ¹H-NMR spectra were recorded on Bruker spectrometers at 400 or 500 MHz. Chemical shifts δ are expressed in ppm using the residual non-deuterated solvent signal as internal standard. The following abbreviations are used: ex, exchangeable; s, singlet; d, doublet; t, triplet; brs, broad signal; m, multiplet. ¹³C-NMR spectrum was recorded at 125.8 MHz, and chemical shifts δ are expressed in ppm using deuterated solvent signal as internal standard. The purity of final compounds, determined to be >98% by UPLC-MS, and low-resolution mass spectra (LRMS) were recorded on a Waters Acquity H-class equipped with a Photodiode array detector and SQ Detector 2 (UPLC-MS) fitted with a reverse phase column (Aquity UPLC® BEH C18 1.7 μm, 2.1×50 mm). High-resolution mass spectra (HRMS) were recorded on a Thermo Fisher Scientific Q-Exactive Plus equipped with a Robotic TriVersa NanoMate Advion.

Lipophilic copper clamp (LCC-12): Dicyandiamide (A10451, Alfa Aesar, 500 mg, 5.94 mmol), 1,12-diaminododecane (A04258, Alfa Aesar, 500 mg, 2.50 mmol) and CuCl₂ (22.201-1, Aldrich 249 mg, 1.85 mmol) were suspended in 6 mL water in a sealed tube and stirred for 1 h, then heated at 80° C. for 48 h. The resulting pink mixture was filtrated, and the solid was re-suspended in water (10 mL). H₂S, generated from dropwise addition of 37% aq. HCl (1.00317.100, Supelco) on FeS (≃100 mesh powder, 17422, Alfa Aesar), was passed into the mixture until it turned black. The black mixture was filtrated, and the filtrate was acidified to pH=5 with a 1M aq. solution of HCl. The solvent was evaporated under reduced pressure. LCC-12 was purified by preparative HPLC (H₂O/CH₃CN/formic acid, 95:5:0.1 to 0:100:0.1) to give the LCC-12 di-formic acid salt as a white powder (280 mg, 24%). ¹H-NMR (500 MHz, DMSO-d₆) δ: 8.80-8.08 (brs, 2H, ex), 8.47 (s, 2H, formate), 7.60-6.78 (brs, 12H, ex), 3.05 (brs, 4H), 1.43 (brs, 4H), 1.32-1.18 (m, 16H) ppm. ¹³C-NMR (125.8 MHz, DMSO-d₆) δ: 167.2 (formate), 160.4, 159.3, 41.3, 29.5 (3C), 29.3, 26.8 ppm. HRMS (ESI+) m/z: calculated for C₁₆H₃₈N₁₀ [M+2H]²⁺ 185.1635, found 185.1635. Metforminyn was synthesized as previously reported (S. Müller, A. Versini, F. Sindikubwabo, G. Belthier, S. Niyomchon, J. Pannequin, L. Grimaud, T. Cañeque, R. Rodriguez, Metformin reveals a mitochondrial copper addiction of mesenchymal cancer cells. PLoS One 13, e0206764 (2018)). ¹H NMR (400 MHz, Methanol-d₆) δ: 3.60 (t, J=7.0 Hz, 2H), 3.09 (s, 3H), 2.50 (td, J=7.0, 3.0 Hz, 2H), 2.36 (t, J=3.0 Hz, 1H) ppm.

High-resolution Mass spectrometry of biguanide:copper(II) complexes (HRMS). HRMS solution were prepared and injected without further dilution. Mixtures (1 mL) were prepared in methanol (980 μL) with LCC-12-2(HCOOH)/K₂CO₃ aq. soln. (1:1) (10 μL, at 10 mM, 1 mM, or 100 μM) and CuCl₂ aq. soln. (10 μL, at 10 mM, 1 mM, or 100 μM) keeping a 1:1 ratio LCC-12/Cu²⁺; or in methanol (800 μL) with metformin-HCl/K₂CO₃ aq. soln. (2:1) (100 μL at 200 mM) and CuCl₂ aq. soln. (100 μL at 100 mM); or in methanol (980 μL) with metformin-HCl/K₂CO₃ aq. soln. (2:1) (10 μL, at 200 mM or 20 mM) and CuCl₂ aq. soln. (10 μL, at 100 mM or 10 mM) keeping a 2:1 ratio metformin/Cu²⁺.

Copper-catalyzed oxidation of NADH. The oxidation kinetics of NADH (N4505, Sigma-Aldrich) was followed by measurement of absorbance at 340 nm using a NanoDrop 2000. Measurements were performed at 26° C. A 10 mM sodium phosphate buffer adjusted to pH=7.2 was used as solvent. Each 500 μL mixture were prepared with NADH (400 μM), imidazole (56750, Sigma-Aldrich, 10 mM), CuSO₄ (451657, Sigma-Aldrich, 4 μM), LCC/K₂CO₃ solution (1:1) (4 μM or 400 μM), metformin/K₂CO₃ solution (2:1) (J63361, Alfa Aesar, 800 μM), as indicated, and a H₂O₂ solution (16911, Sigma-Aldrich, 2 mM from an aq. solution 25-35% in H₂O₂) added at the reaction start time. The concentration of NADH was calculated from the measured absorbance at 340 nm and a molar extinction coefficient of 5.5×10³ L mol⁻¹ cm⁻¹.

NADH/NAD⁺ measurements in macrophages. NAD⁺ and NADH levels were measured using an NAD⁺/NADH fluorometric assay (Abcam, ab176723) according to the manufacturer's protocol. In brief, at least 500.000 cells were harvested per condition. Floating cells were harvested and adherent cells were washed with 1×PBS. Adherent cells were incubated with 1×PBS with 10 mM EDTA and then scraped and pooled together with the harvested floating cells. Cells were subsequently washed with ice-cold 1×PBS and counted. Cells were centrifuged at 1500 rpm for 5 min and the supernatant discarded. The pellet was then resuspended in 100 μL lysis buffer (kit component) and incubated at 37° C. for 15 min. Standards were prepared according to the manufacturer's protocol. NAD⁺ and NADH extraction solutions as well as NAD⁺/NADH control solutions (kit components) were added and incubated at 37° C. for 15 min at a volume of 15 μL sample to 15 μL of the respective buffers. The reactions were stopped using 15 μL of respective buffer. Finally, 75 μL of NAD⁺/NADH reaction mixture (NAD⁺/NADH recycling enzyme mixture and sensor buffer, kit components) were added and the resulting mixtures incubated for 1 h at room temperature. Fluorescence intensities (ex. 540 nm; em. 590 nm) were recorded using a Perkin Elmer Wallac 1420 Victor2 Microplate Reader, and data were normalized against cell numbers. Values were derived from the standard curve of each experiment.

Cytokine measurements. [IL6] and [IL10] were measured in cell culture supernatants using V-Plex validated immunoassay (MSD, Rockville, MD, US). The kit was run according to the manufacturer's protocol and the chemiluminescence signal was measured on a Sector Imager 2400 (MSD).

Murine model of LPS-induced severe inflammation. Animal work was conducted at Fidelta Ltd according to 2010/63/EU and National legislation regulating the use of laboratory animals in scientific research and for other purposes (Official Gazette 55/13). An Institutional Committee on Animal Research Ethics (CARE-Zg) oversaw that animal-related procedures were not compromising the animal welfare. LPS (Sigma-Aldrich, L2630, 20 mg/kg) was injected intraperitoneally to male BALB/c mice (8 weeks-old). LCC-12 (0.3 mg/kg, IP, n=10) or vehicle (0.9% NaCl, 10 mL/kg, IP, n=10) were injected 2 h prior LPS challenge, then 24 h, 48 h, 72 h and 96 h post challenge. Dexamethasone (10 mg/kg, PO, n=10) was given 1 h prior LPS challenge. Incidence of mortality was monitored every 4 h up to 48 h, then twice daily.

Murine model of sepsis using cecal ligation and puncture. All animal-related research is conducted in accordance with 2010/63/EU and National legislation regulating the use of laboratory animals in scientific research and for other purposes (Official Gazette 55/13). An Institutional Committee on Animal Research Ethics (CEEA-047) oversees that animal-related procedures are not compromising the animal welfare. 9 weeks-old male BALB/c mice were used for these experiments. Animals were anesthetized by isoflurane (Forene). After abdominal incision, the cecum was ligated, punctured with a gauge needle (25G or 21G), and a small amount of fecal matter was released. After the cecum was returned to the abdomen, the abdominal cavity was closed in two layers and the mice were resuscitated with 30 mL/kg body weight of saline (0.9% NaCl) administered subcutaneously. For the sham group, after abdominal incision, the cecum was manipulated but was neither ligated nor punctured. After the cecum was returned to the abdomen, the abdominal cavity was closed in two layers and the mice were resuscitated with 30 mL/kg body weight of saline administered subcutaneously. LCC-12 (0.3 mg/kg, IP) was administered at 0.3 mg/kg dose 4 h, 24 h, 48 h, 72 h and 96 h following CLP creation. Mortality incidence was monitored every 2 h up to 120 h (except from 10 pm to 6 am) post CLP creation. Dexamethasone was administered intraperitoneally at 1 mg/kg dose 5-minutes prior CLP creation.

Statistical analyses. All results are presented as mean values±standard error of the mean (SEM) unless stated otherwise. PRISM 8 software was used to calculate p-values using a Mann-Whitney test or Kruskal-Wallis test with Dunn's post-test for multiple comparisons as indicated. PRISM 8 software or R programming language was used to generate graphical representations of quantifications unless stated otherwise. Sample sizes (n) are indicated in the figure legends.

Data and Code Availability

RNA-seq data are available on the National Center for Biotechnology Information website with accession reference GSE160864 (go to: https://www.ncbi.nlm.nih.gov/geo/query/acc.cgi?acc=GSE160864; enter token wvqxgwgojxcdboz into the box). Analysis scripts are available at https://github.com/bioinfo-pf-curie/MDMmetals.

### Example 3: Biguanides Affect the Activation of Immune Cells

LCC-12 decreases the inflammatory profile of macrophages as illustrated in Example 2. The inventors equally reported an impact of LCC-12 on other inflammatory cells (FIG. 6). LCC-12 decreases the activation of lymphocytes, dendritic cells and monocytes. To note, LCC-12 does not impact the activation of neutrophils in vitro. LCC-12 targets preferentially macrophage activation but it also affects other inflammatory cells. This strengthens the fact that LCC-12 is a novel strategy to control inflammation. Altogether, these data also illustrate the general nature of this copper signaling pathway, identifying CD44 as a regulator of cell plasticity.

Materials and Methods

CD4 Lymphocytes. Peripheral blood samples were collected from healthy donors (Etablissement Français du Sang). CD4 lymphocytes were isolated by negative magnetic sorting using microbeads according to the manufacturer's instructions (Miltenyi Biotec, 130-096-533) and cultured in RPM11640 supplemented with glutamine and 10% fetal bovine serum. CD4 lymphocytes were activated for 48 h using CD3/CD28 antibodies (2.5 μg/mL), in presence of LCC-12 (10 μM). The activation status of the lymphocytes was assessed by measuring CD25 and CD69 surface markers by flow cytometry.

CD8 Lymphocytes. Peripheral blood samples were collected from healthy donors (Etablissement Français du Sang). CD8 lymphocytes were isolated by negative magnetic sorting using microbeads according to the manufacturer's instructions (Miltenyi Biotec, 130-096-495) and cultured in RPM11640 supplemented with glutamine and 10% fetal bovine serum. CD8 lymphocytes were activated 48 h using CD3/CD28 antibodies (2.5 μg/mL), in presence of LCC-12 (10 μM). The activation status of the lymphocytes was assessed by measuring CD25 and CD69 surface markers by flow cytometry.

Neutrophils. Peripheral blood samples were collected from healthy donors (Etablissement Français du Sang). Then, red cells in whole blood samples were lysed (ebioscience 10×RBC lysis buffer, 00-4300-54). The remaining cells were cultured in RPMI 1640 supplemented with glutamine, and 2% human serum, and activated 1 h with LPS (2 μg/mL) in presence of LCC-12 (10 μM). The granulocytes/neutrophils population was determined by flow cytometry using FSC, SSC and CD15 surface marker. The activation status of the granulocytes was assessed by measuring CD64 and CD66b surface markers by flow cytometry.

Monocytes. Peripheral blood samples were collected from healthy donors (Etablissement Français du Sang). Pan monocytes were isolated by negative magnetic sorting using microbeads according to the manufacturer's instructions (Miltenyi Biotec, 130-096-537), and cultured in RPMI 1640 supplemented with glutamine, 10% fetal bovine serum. Monocytes were treated with lipopolysaccharides (LPS, 100 ng/mL, 24 h) to generate activated monocytes and were co-treated with LCC-12 (in-house, 10 μM, 24 h). The activation status of the monocytes was assessed by measuring CD25 and CD80 surface markers by flow cytometry.

Dendritic cells. Peripheral blood samples were collected from healthy donors (Etablissement Français du Sang). Pan monocytes were isolated by negative magnetic sorting using microbeads according to the manufacturer's instructions (Miltenyi Biotec, 130-096-537), and cultured in RPMI 1640 supplemented with glutamine, 10% fetal bovine serum and treated with granulocyte-macrophage colony-stimulating factor (GM-CSF, 100 ng/mL) and IL-4 (10 ng/mL) to induce differentiation into dendritic cells (DC). At day 5 of differentiation, DC were treated with lipopolysaccharides (LPS, 100 ng/mL, 24 h) to generate activated DC and were co-treated with LCC-12 (in-house, 10 μM, 24 h). The activation status of the dendritic cells was assessed by measuring CD40, CD83, CD80 and CD86 surface markers by flow cytometry.

### Example 4: Effect of a Series of LCCs on Macrophage Activation and on the Lymphoma Cell Line U937

The inventors tested a series of LCC molecules on macrophage activation using 10 μM or 1 μM of compound (as indicated in the table). They also assessed the half maximal inhibitory concentration (IC₅₀) of cell viability on the lymphoma cell line U-937. Depending on linker length and overall length, there was a varying degree of potency on macrophage activation as well as a range of IC₅₀ values in the nanomolar to micromolar range. This data highlights the potential of lead structure optimization of this series of biguanides.

| | Effect on CD86 macrophage activation | | |
|---|---|---|---|
| | marker (Flow cytometry)(All LCCs at | | IC₅₀ |
| | 10 μM or 1 μM if indicated and Met at | | (μM) |
| | 10 mM)(% of activation marker | | in |
| | compared to its level in activated | | U937 |
| Compound | macrophages) | | cells |
| Metformin | 51 | | 10000 |
| | 59 | | |
| | 62 | | |
| | 70 | | |
| | 56 | | |
| | 57 | | |
| | 53 | | |
| | 53 | | |
| LCC-7 | | | 300 |
| LCC-8 | 77 | | 200 |
| LCC-9 | | | 60 |
| LCC-10 | | | 20 |
| LCC-12 | 25 | 1 μM | 2 |
| | 29 | 83 | |
| | 26 | 51 | |
| | 25 | | |
| | 18 | | |
| | 24 | | |
| | 22 | | |
| | 20 | | |
| | 26 | | |
| | 27 | | |
| LCC-12-15N | | | |
| LCC-8Me | 89 | | 150 |
| LCC-12Me | 24 | | 6 |
| | 15 | | |
| | 17 | | |
| LCC-8,3 | 59 | | 2 |
| | 39 | | |
| LCC-10,2 | 32 | 1 μM | 1 |
| | 29 | 54 | |
| | | 58 | |
| LCC-10,3 | 22 | 1 μM | 0.6 |
| | | 38 | |
| | | 43 | |
| LCC-10,5 | | | 0.3 |
| LCC-12,2 | | | 0.4 |
| LCC-12,3 | | | 0.3 |
| LCC-12,4 | | 1 μM | 0.3 |
| LCC-12,6 | | | 0.4 |
| LCC-12Me, | | 1 μM | |
| 1,1 (A) | | 35 | |
| | | 34 | |
| LCC-12Me, | | 1 uM | |
| 2 (B) | | 34 | |
| LCC-12-click | | | 0.4 |
| Coronaformin- | | | 3 |
| 8,8 | | | |
| Coronaformin- | 50 | | 1 |
| 10,10 | 45 | | |
| Coronaformin- | | 1 μM | 0.5 |
| 12,12 | | 62 | |
| | | 55 | |

Materials and Methods

Cell culture. U-937 cells were grown in an incubator equilibrated at 37° C. with 5% CO₂, grown to confluence and split with Trypsin/EDTA (Gibco, TRYPGIB01) once or twice a week according to confluence. U-937 (ATCC, HTB-132, sex: female) were cultured in RPMI 1640 GLUTAMAX (ThermoFisher Scientific, 61870044) supplemented with 10% Fetal Bovine Serum (FBS, Gibco, 10270-106) and Penicillin-Streptomycin mixture (BioWhittaker/Lonza, DE17-602E). U-937 cells and were co-treated with metformin (Met, 1,1-dimethylbiguanid hydrochloride, Alfa Aesar, J63361, 10 mM, 24 h) or different LCC compounds (in-house, 10 μM or 1 μM, 24 h) as indicated. Peripheral blood samples were collected from distinct healthy donors (Etablissement Français du Sang). Pan monocytes were isolated by negative magnetic sorting using microbeads according to the manufacturer's instructions (Miltenyi Biotec, 130-096-537), and cultured in RPMI 1640 supplemented with glutamine (Thermo Fisher Scientific, 61870010), 10% fetal bovine serum and exposed to granulocyte-macrophage colony-stimulating factor (GM-CSF, Miltenyi Biotec, 130-093-866, 100 ng/mL) to induce differentiation into macrophages (MDM). At day 5 of differentiation, MDM were treated with lipopolysaccharides (LPS, InvivoGen, tlrl-3pelps, 100 ng/mL, 24 h) and interferon-γ (IFNγ, Miltenyi Biotec, 130-096-484, 20 ng/mL, 24 h) to generate activated MDM (act. MDM) and were co-treated with metformin (Met, 1,1-dimethylbiguanid hydrochloride, Alfa Aesar, J63361, 10 mM, 24 h) or different LCC compounds (in-house, 10 μM or 1 μM, 24 h) as indicated.

Flow cytometry. Cells were washed with ice-cold 1×PBS, incubated with Fc block (Human TruStain FcX, Biolegend, 422302, 1/20) for 15 min, incubated with antibodies for 20 min at 4° C. and washed before analysis using a BD LSRFortessa X-20. Macrophages were analyzed with an antibody panel consisting of antibodies against the following cell surface proteins: CD44, CD80, CD86 and TfR1. The data were analyzed with FlowJo software v. 10.0.00003. Flow cytometry analysis of lysosomal iron content: Lysosomal iron was monitored by incubating cells at 37° C. with 5% CO₂ in medium containing RhoNox-M (in-house, 1 μM, 1 h), before flow analysis of fluorescence intensity.

Cell viability assay (IC₅₀). Cell viability assay was carried out by plating 1000 cells/well in 96-well plates. Cells were treated for 72 h in a range between to 25 nM and 100 mM using serial dilutions. The inventors followed the manufacturer's protocol. In brief, CellTiter-Blue® reagent (G8081, Promega) was added after 72 h treatment and cells were incubated for 3 h before recording fluorescence intensities (λ_{ex.} 560/20 nm; λ_{em.} 590/10 nm) using a Perkin Elmer Wallac 1420 Victor2 Microplate Reader.

### Example 5: Effect on Cancer Stem Cells and Cancer Cell Plasticity

Cancer stem cells (CSC) represent a subpopulation in many cancers and resistance to therapy as well as metastatic dissemination and relapse can be attributed to these cells. Cancer cells can acquire a cell stem cell state without genetic mutations, but rather epigenetic alterations, i.e. cell plasticity. The phenomenon of cell plasticity in cancer has been extensively studied for the epithelial-to-mesenchymal transition (EMT), whereby the mesenchymal state has characteristics attributed to cancer stem cells. Since biguanides affect cell plasticity, the inventors investigated their effect on cancer cell plasticity. Using a well-established model of breast CSCs, namely HMLER CD44^{high}/CD241^{low} (Morel et. al, 2008, PlosONE, 3, e2888), the inventors found IC₅₀-values much lower than metformin depending on the LCC tested (see table). LCC-8, LCC-10 and LCC-12 show an improved IC50, by respectively about 100 fold, about 400 fold and more than 2800 fold.

| | IC₅₀ against |
|---|---|
| | HMLER |
| Compound | CD44^{high}/CD24^{low} |
| Metformin | 22.6 |
| LCC-4 | 22.3 |
| LCC-8 | 0.240 |
| LCC-10 | 0.058 |
| LCC-12 | 0.008 |

IC₅₀ are expressed in mM.

Furthermore, the inventors used the human breast cancer cell line MCF7, the mouse pancreatic cancer cell line FC1242 and the prostate cancer cell line DU-145, where the mesenchymal state of EMT can be induced using TGF-β or OSM depending on the cells. LCC-12 showed lower IC₅₀-values in cells in the mesenchymal state compared to the epithelial counterpart (FIG. 7). Interestingly, the inverse was observed for one of the standard of care chemotherapies used in pancreatic ductal adenocarcinoma (PDAC), namely FOLFORINOX (with the active ingredients oxaliplatin, irinotecan, 5-FU), and LCC-12 compared favorably to the standard of care. Furthermore, using FC1242, MCF7 and primary human lung circulating tumor cells, the inventors observed that upon biochemical stimulation (TFG-β or OSM) cells in the mesenchymal state had increased levels of CD44, SOD2 and copper (FIG. 8A-C). Importantly, LCC-12 treatment antagonized EMT induction as attested by levels of the epithelial marker E-cadherin and the mesenchymal markers Fibronectin and Slug (FIG. 8D). Taken together, these data highlight that biguanides can selectively target the cancer stem cell niche and/or block EMT. Thus, the LCC family of compounds reduces cell plasticity, including activation of inflammatory and immune cells and plasticity of cancer cells, for instance epithelial-to-mesenchymal transition (EMT). For the treatment of cancer, blocking EMT desensitizes cells to cytotoxic agents.

Materials and Methods

Cell viability assay (IC₅₀). Cell viability assay was carried out by plating 1000 cells/well in 96-well plates. Cells were treated for 72 h in a range between to 25 nM and 100 mM using serial dilutions. The inventors followed the manufacturer's protocol. In brief, CellTiter-Blue® reagent (G8081, Promega) was added after 72 h treatment and cells were incubated for 3 h before recording fluorescence intensities (λ_{ex.} 560/20 nm; λ_{em.} 590/10 nm) using a Perkin Elmer Wallac 1420 Victor2 Microplate Reader.

Cell culture. MCF7 (ATCC) cells, DU-145 (ATCC) cells and FC1245 cells were cultured in Dulbecco's Modified Eagle Medium GlutaMAX (DMEM, ThermoFisher Scientific, 61965059) supplemented with 10% Fetal Bovine Serum (FBS, Gibco, 10270-106) and Penicillin-Streptomycin mixture (BioWhittaker/Lonza, DE17-602E) unless stated otherwise. Primary lung circulating tumor cells (Celprogen, 36107-34CTC, Lot 219411, sex: female) were grown using stem cell complete media (Celprogen, M36102-29PS) until the third passage. Circulating cancer cells were grown in stem cell ECM T75-flasks (Celprogen, E36102-29-T75) and ECM 6-well plates (Celprogen, E36102-29-6Well). HMLER cells (sex: female) naturally repressing E-cadherin, obtained from human mammary epithelial cells infected with a retrovirus carrying hTERT, SV40 and the oncogenic allele H-rasV12, and HMLER CD44 and TFRC ko clones were cultured in DMEM/F12 (Thermo Fisher Scientific, 31331093) supplemented with 10% FBS (Thermo Fisher Scientific, 10270106), 10 μg/mL insulin (Sigma-Aldrich, 10516), 0.5 μg/mL hydrocortisone (Sigma-Aldrich, H₀₈₈₈) and 0.5 μg/mL puromycin (Life Technologies, A11138-02), unless stated otherwise. HMLER CD44^{high} cells were also supplemented with 10 ng/mL EGF (Miltenyi Biotech, 130-097-750).

Flow cytometry. Cells were washed twice with ice-cold 1×PBS and suspended in incubation buffer prior to being analysed by flow cytometry. For each condition, at least 10,000 cells were counted. Data were recorded on a BD Accuri C6 (BD Biosciences) and processed using Cell Quest (BD Biosciences) and FlowJo (FLOWJO, LLC).

Western Blotting. Cells were treated as indicated and then washed with 1×PBS. Proteins were solubilized in 2× Laemmli buffer containing benzonase (VWR, 70664-3, 1:100), extracts were incubated at 37° C. for 1 h, and quantified using a NanoDrop 2000 spectrophotometer (ThermoFisher Scientific). Protein lysates were resolved by SDS-PAGE electrophoresis (Invitrogen sure-lock system and Nu-PAGE 4-12% Bis-Tris precast gels) and transferred onto nitrocellulose (Amersham Protran 0.45 μm) membranes using a Trans-Blot SD semi-dry electrophoretic transfer cell (Bio-rad). Membranes were blocked with 5% non-fat skimmed milk powder in 0.1% Tween-20/1×PBS for 1 h. Blots were then probed with the relevant primary antibodies in 5% BSA, 0.1% Tween-20/1×PBS at 4° C. overnight with gentle motion. Membranes were washed with 0.1% Tween-20/1×PBS three times and incubated with horseradish peroxidase conjugated secondary antibodies (Jackson Laboratories) in 5% non-fat skimmed milk powder, 0.1% Tween-20/1×PBS for 1 h at room temperature and washed three times with 0.1% Tween-20/1×PBS. Antigens were detected using the SuperSignal West Pico PLUS chemiluminescent detection kits (ThermoFisher Scientific, 34580 and 34096). Signals were recorded using a Fusion Solo S Imaging System (Vilber) and quantified as indicated using ImageJ. Antibodies used were: SOD2 (Abcam, ab13534), E-cadherin (Cell Signaling, 3195), γ-Tubulin (Sigma-Aldrich, T5326), Fibronectin (Sigma-Aldrich, F1141-1MG), SLUG (Cell Signaling, 9585S).

### Example 6: Biguanides Show Efficacy on Biopsy-Derived Organoids of Pancreatic Ductal Adenocarcinoma (PDAC)

The inventors tested LCC-12 on biopsy-derived organoids of PDAC, and found that the molecule had an efficacy in the low micromolar range on a variety of organoids (FIG. 9).

Materials and Methods

Biopsy-derived pancreatic organoid (BDPO) generation. BDPOs were obtained from endoscopic ultrasound-guided fine-needle aspirations (EUS-FNA) from patients with PDAC included under the PaCaOmics clinical trial (ClinicalTrials.gov: NCT01692873) after approval by the Paoli-Calmettes hospital ethics committee and following patient informed consent. Cultures were established as previously described. Briefly, PDAC biopsies were slightly digested with the Tumor Dissociation Kit (Miltenyi Biotec) at 37° C. for 5 minutes. The pancreatic tissue slurry was transferred into a tissue strainer 100 μm and was placed into 12-well plates coated with 150 μL GFR matrigel (Corning, Boulogne-Billancourt, France). The samples cultured with Pancreatic Organoid Feeding Media (POFM) consisted of Advanced DMEM/F12 supplemented with 10 mM HEPES (Thermo Fisher Scientifics, Courtaboeuf, France); 1×Glutamax (Thermo-Fisher Scientifics); penicillin/streptomycin (Thermo-Fisher Scientifics); 100 ng/mL Animal-Free Recombinant Human FGF10 (Peprotech, Peprotech, Neuilly-Sur-Seine, France); 50 ng/mL Animal-Free Recombinant Human EGF (Peprotech); 100 ng/mL Recombinant Human Noggin (Biotechne, Bio-Techne, Rennes, France); Wnt3a-conditioned medium (30% v/v); RSPO1-conditioned medium (10% v/v); 10 nM human Gastrin 1 (Sigma-Aldrich Lyon, France); 10 mM nicotinamide (Sigma Aldrich); 1.25 mM N-acetylcysteine (Sigma Aldrich); 1×B27 (Invitrogen, (Invitrogen, Villebon sur Yvette, France); 500 nM A83-01 (Tocris, Noyal Chätillon sur Seiche, France); 10.5 μM Y27632 (Tocris). The plates were incubated at 37° C. in a 5% CO₂ incubator, and the media were changed every 3 or 4 days. For routine passages BDPOs were disaggregated with accutase (Thermo Fisher Scientific) and re-plated as needed.

Chemograms on BDPO. BDPOs were disaggregated with accutase (Thermo Fisher Scientific), and 1,000 cells/well were plated in two 96-well round bottom ultra-low plates (Corning) with the medium described above. 24 hours later, one plate was used directly for RNA preparation (Time 0 transcriptome) and on the other the medium was supplemented with increasing concentrations of each drug, 72 hours later cell viability was measured with CellTiter-Glo 3D (Promega) reagent quantified using the plate reader Tristar LB941 (Berthold Technologies). Values were normalized and expressed as the percentage of the control (vehicle), which represents 100% of normalized fluorescence. Increasing concentrations of drugs were used. Each experiment was repeated at least twice.

### Example 7: Biguanides Target Mitochondria and Mitochondrial Metabolism

To gain further insights into the mechanism of action (MoA) of LCC-12, the inventors employed nanoscale secondary ion mass spectrometry (NanoSIMS) imaging, which allows for qualitative assessment of specific isotope distributions of elements in a cell. To this end they employed the isotopologue ¹⁵N-¹³C-LCC-12, which gave rise to a similar NanoSIMS imaging pattern as did ¹⁹⁷Au loaded onto an antibody against cytochrome c, suggesting that LCC-12 targets mitochondria (FIGS. 10A and B). To support this finding, the inventors developed a biologically active alkyne-containing analog that can be chemically labeled in cells by means of click chemistry and then detected by fluorescence microscopy. In aMDM, the labeled small molecule was detected as cytoplasmic puncta that localized in the vicinity of cytochrome c, thus confirming accumulation of LCC-12 in mitochondria (FIG. 10C-D). The fluorescence intensity of the labeled small molecule was reduced upon co-treatment with carbonyl cyanide m-chlorophenyl hydrazone (CCCP), a compound that dissipates the mitochondrial proton gradient (FIG. 10E). This indicated that LCC-12 accumulation in mitochondria is driven by its protonation state.

Labeling small molecules in cells by means of click chemistry requires a copper(I) catalyst generated in situ by adding copper(II) and ascorbate (Asc) as a reducing agent. Given that the investigation converged towards mitochondrial copper(II) as a mechanistic target of LCC-12, the inventors investigated whether the natural abundance of mitochondrial copper(II) they found in aMDM could allow for click labeling without the need to experimentally add the metal catalyst. The inventors found that fluorescent labeling of the clickable analog of LCC-12 used at a concentration of 100 nM, which is 100-fold lower than the biologically active dose of LCC-12, occurred in the absence of exogenous copper, leading to a fluorescent signal in aMDM that colocalized with mitotracker. Importantly, such a staining was observed only when MDM were activated (FIG. 10F), and when ascorbate was experimentally added (FIG. 10G). Furthermore, the fluorescence intensity was substantially reduced when a 100-fold molar excess of LCC-12 competitor was added. To gain further insights into mechanisms at work, the inventors isolated mitochondria and quantified their metal content by ICP-MS. Importantly, mitochondrial copper levels were higher in aMDM compared to naMDM (FIG. 10H). Interestingly, the inventors also observed an increase of manganese in mitochondria of aMDM, whereas the content of other metals studied was not significantly increased. Taken together, these data support the idea that mitochondrial copper(II) is a key regulator of macrophage activation and a mechanistic target of LCC-12. This is further supported by the lack of efficacy of D-Pen and ATTM, which exert their activity through the targeting of copper(I) and for which a preferential mitochondrial targeting has not been documented.

To delve further into the mechanism at work of biguanides and mitochondrial copper targeting, the inventors investigated the reactions underpinning copper-catalyzed interconversion of NAD(H). In line with the proposed mechanism, they found that mitochondrial superoxide dismutase 2 (SOD2) levels increase during macrophage activation, an enzyme that interconverts superoxide to hydrogen peroxidase (FIGS. 11A and B). Concomitantly, mitochondrial hydrogen peroxide was elevated in aMDM compared to MDM (FIG. 11C). The higher abundance of copper(II) and hydrogen peroxide in mitochondria of aMDM compared to naMDM prompted the inventors to investigate the biological relevance of such a reaction in inflammatory macrophages. To this end, they quantified levels of mitochondrial NADH and NAD⁺ in aMDM by mass spectrometry-based metabolomics. Mitochondrial NADH levels were higher whereas NAD⁺ levels were lower in aMDM compared to naMDM, suggesting an enhanced activity of mitochondrial enzymes reliant on NAD⁺. In agreement with data obtained from our cell-free system, treating macrophages with LCC-12 during activation led to a decrease of both mitochondrial NAD⁺ and NADH (FIG. 11D). This is consistent with the idea that copper(II) catalyzes the reduction of hydrogen peroxide by NADH to produce NAD⁺ in cells and that biguanides can interfere with this redox cycling, leading instead to other oxidation byproducts. Notably, NADH and copper were found in mitochondria of aMDM at an estimated substrate/catalyst ratio of 2:1, which is even more favorable for this reaction than the 20:1 ratio used in the cell-free system. Quantitative metabolomics analysis of total cellular extracts indicated that macrophage activation was characterized by altered levels of several metabolites whose production depend on NAD(H) (FIGS. 11E and F).

Materials and Methods

Mitochondrial extraction. Mitochondria were isolated using the Qproteome Mitochondria Isolation Kit (Qiagen, 37612) according to the manufacturer's protocol. In brief, cells were washed and centrifuged at 500× g for 10 min and the supernatant was removed. Cells were then washed with a solution of 0.9% NaCl (Sigma-Aldrich, 57653-250G) and resuspended in ice-cold Lysis Buffer and incubated at 4° C. for 10 min. The lysate was then centrifuged at 1000×g for 10 min at 4° C. and the supernatant carefully removed. Subsequently, the cell pellet was resuspended in disruption buffer. Complete cell disruption was obtained by using a dounce homogenizer (mitochondria for ICP-MS) or a blunt-ended needle and a syringe (mitochondria for metabolomics). The lysate was then centrifuged at 1000× g for 10 min at 4° C. and the supernatant transferred to a clean tube. The supernatant was then centrifuged at 6000× g for 10 min at 4° C. to obtain pellets containing mitochondria.

Mitochondrial H₂O₂ content. Mitochondrial H₂O₂ was monitored by incubating cells at 37° C. with 5% CO₂ in medium containing Mito-PY1 (R&D Systems, #4428, 5 μM, during the last 24 h), before flow analysis of fluorescence intensity.

Quantitative metabolomics. In a typical experiment 1.5 million cells were used for total extracts and 15 million cells for mitochondrial extracts. Cells were harvested and the supernatant removed to generate the corresponding cell pellets. Subsequently, pellets were dried and dry pellets were supplemented with 300 μl methanol, vortexed 5 min and centrifuged (10 min at 15000 g, 4° C.). Then, the upper phase of the supernatant was split into two parts: 150 μL were used for a gas chromatography coupled by mass spectrometry (GC/MS) experiment in microtubes and the remaining 150 μL were used for Ultra High Pressure Liquid Chromatography coupled by Mass Spectrometry (UHPLC/MS). For the GC-MS aliquots, supernatants were completely evaporated from the sample. 50 μL of methoxyamine (20 mg/mL in pyridine) were added to the dried extracts, then stored at room temperature in the dark for 16 h. The following day, 80 μL of MSTF (A-Methyl-N-(trimethylsilyl) trifluoroacetamide) were added and final derivatization occurred at 40° C. for 30 min. Samples were then transferred into vials and directly injected for GC-MS analysis. For the UHPLC-MS aliquots, 150 μL were dried in microtubes at 40° C. in a pneumatically-assisted concentrator (Techne DB3, Staffordshire, UK). The dried UHPLC-MS extracts were solubilized with 200 μL of MilliQ water. Aliquots for analysis were transferred into LC vials and injected into UHPLC-MS or kept at −80° C. until injection. Widely-targeted analysis of intracellular metabolites gas chromatography (GC) coupled to a triple quadrupole (QQQ) mass spectrometer: GC-MS/MS method was performed on a 7890A gas chromatography (Agilent Technologies, Waldbronn, Germany) coupled to a triple quadrupole 7000C (Agilent Technologies, Waldbronn, Germany) equipped with a High sensitivity electronic impact source (EI) operating in positive mode (Viltard et al., 2019). Peak detection and integration of the analytes were performed using the Agilent Mass Hunter quantitative software (B.07.01). Targeted analysis of nucleotides and cofactors by ion pairing ultra-high performance liquid chromatography (UHPLC) coupled to a Triple Quadrupole (QQQ) mass spectrometer: Targeted analysis was performed on a RRLC 1290 system (Agilent Technologies, Waldbronn, Germany) coupled to a Triple Quadrupole 6470 (Agilent Technologies) equipped with an electrospray source operating in both negative and positive modes. Gas temperature was set to 350° C. with a gas flow of 12 L/min. Capillary voltage was set to 5 kV in positive mode and 4.5 kV in negative mode. 10 μL of sample were injected on a Column Zorbax Eclipse XDB-C18 (100 mm×2.1 mm particle size 1.8 μm) from Agilent technologies, protected by a guard column XDB-C18 (5 mm×2.1 mm particle size 1.8 μm) and heated at 40° C. by a pelletier oven. The gradient mobile phase consisted of water with 2 mM of dibutylamine acetate concentrate (DBAA) (A) and acetonitrile (B). Flow rate was set to 0.4 mL/min and an initial gradient of 90% phase A and 10% phase B, which was maintained for 3 min. Molecules were then eluted using a gradient from 10% to 95% phase B over 1 min. The column was washed using 95% mobile phase B for 2 minutes and equilibrated using 10% mobile phase B for 1 min and the autosampler was kept at 4° C. Scan mode used was the MRM for biological samples. Peak detection and integration of the analytes were performed using the Agilent Mass Hunter quantitative software (B.10.1). Pseudo-targeted analysis of intracellular metabolites by ultra-high performance liquid chromatography (UHPLC) coupled to a Q-Exactive mass spectrometer. Reversed phase acetonitrile method: The profiling experiment was performed with a Dionex Ultimate 3000 UHPLC system (Thermo Scientific) coupled to a Q-Exactive (Thermo Scientific) equipped with an electrospray source operating in both positive and negative modes and full scan mode from 100 to 1200 m/z. The Q-Exactive parameters were: sheath gas flow rate 55 au, auxiliary gas flow rate 15 au, spray voltage 3.3 kV, capillary temperature 300° C., S-Lens RF level 55 V. The mass spectrometer was calibrated with sodium acetate solution dedicated to low mass calibration. 10 μL of sample were injected on a SB-Aq column (100 mm×2.1 mm particle size 1.8 am) from Agilent Technologies, protected by a guard column XDB-C18 (5 mm×2.1 mm particle size 1.8 am) and heated at 40° C. by a pelletier oven. The gradient mobile phase consisted of water with 0.2% acetic acid (A) and acetonitrile (B). The flow rate was set to 0.3 mL/min. The initial condition was 98% phase A and 2% phase B. Molecules were then eluted using a gradient from 2% to 95% phase B for 22 min. The column was washed using 95% mobile phase B for 2 for min and equilibrated using 2% mobile phase B for 4 min. The autosampler was kept at 4° C. Peak detection and integration were performed using the Thermo Xcalibur quantitative software (2.1) (Viltard et al., 2019, Aging 11, 4783-4800).

Nanoscale secondary ion mass spectrometry (NanoSIMS). aMDM were grown on coated cover slips and treated with 10 μM ¹⁵N-¹³C-LCC-12 for 3 h. Subsequently, cells were washed twice with 1×PBS, once with 0.1 M cacodylate buffer (LFG Distribution, 11653) and then fixed with 2% paraformaldehyde in 0.1 M cacodylate buffer for 20 min. Then, cells were washed three times with 0.1 M cacodylate buffer for 5 min and permeabilized with 0.1% Triton-X in 0.1 M cacodylate buffer for 5 min. Subsequently, cells were washed three times with 0.1 M cacodylate buffer and blocking buffer (2% BSA, 0.1% Tween in 0.1 M cacodylate buffer) was added for 20 min. Primary antibody (1:400) was added for 1 h in blocking buffer. Then, cells were washed three times with 0.1 M cacodylate buffer and the 10 nM gold-nanoparticle-loaded secondary antibody (1:50) was added in blocking buffer for 1 h. Cells were washed three times with 0.1 M cacodylate buffer and treated with 1% OSO₄ (Electron Microscopy Sciences, 19152) in 0.1 M cacodylate buffer for 1 h. Cover slips with samples were washed three times for 10 min with Milli-Q water. Subsequently, cells were dehydrated with sequential EtOH solutions each for 10 min each: 50%, 70%, 2×90%, 3×100% (dried over molecular sieves, Sigma-Aldrich, 69833). Samples were then coated with a 1:1 mixture of resin (Electron Microscopy Sciences, dodecenylsuccinic anhydride, 13710, methyl nadic anhydride, 19000, DMP-30, 13600 and LADD research industries: LX112 resin, 21310) and dry EtOH for 1 h. Then, samples were embedded in pure resin for 1 h. Embedding capsules (Electron Microscopy Sciences, 69910-10) were filled with resin, inverted onto the cover slides and placed in an oven at 56° C. for 24 h. 0.2 am sections were prepared using a Leica Ultracut UCT microtome. Sample sections were deposited onto a clean silicon chip (Institute for Electronic Fundamentals/CNRS and University Paris Sud) and dried upon exposure to air before being introduced into the NanoSIMS-50 ion microprobe (Cameca, Gennevilliers, France). A Cs⁺ primary ion was employed to generate negative secondary ion from the sample surface. The probe steps over the image field and the signal of selected secondary ion species were recorded pixel-by-pixel to create 2D images. Image of ¹²C¹⁴N⁻ was recorded to provide the anatomic structure of the cells, while the one of ³¹P⁻ highlights the location of cell nucleus. The cellular distribution of ¹⁵N-label was imaged by measuring the excess in ¹²C¹⁵N⁻ to ¹²C¹⁴N⁻ ratio with respect to the natural abundance level (0.0037), and the one for antibody with gold staining targeting mitochondria was performed by detecting directly ¹⁹⁷Au⁻ ion. When detecting ¹²C¹⁵N⁻ ion, appropriate mass resolution power was required to discriminate abundant ¹³C¹⁴N⁻ isobaric ions (with an M/ΔM of 4272). For each image recording process, multiframe acquisition mode was applied and hundreds of image planes were recorded. The overall acquisition time corresponding to the ¹⁵N image was 12 h and 6 h 30 mins for the ¹⁹⁷Au image. During image processing with ImageJ, the successive image planes were properly aligned using TomoJ plugin (Messaoudii et al., 2007, Bioinformatics 8, 288-296), so as to correct the slight primary beam shift during long hours of acquisition. A summed image was then obtained with improved statistics. Further, for the ¹²C¹⁵N⁻ to ¹²C¹⁴N⁻ ratio map, an HSI (Hue-Saturation-Intensity) color image was generated using OpenMIMS for display with increased significance (Lechene et al., 2006, J. Biol. 5, 20). The hue corresponds to the absolute ¹⁵N/¹⁴N ratio value, and the intensity at a given hue is an index of the statistical reliability.

Synthesis. Clickable Lipophilic copper clamp 12: Bis-(cyanoguanidino)dodecane (227 mg, 0.60 mmol) and but-3-yne-1-amine hydrochloride (EN300-76524, Enamine, 126 mg, 1.20 mmol) were mixed together in a sealed tube and heated at 150° C. without solvent for 4 h. After cooling to rt, the mixture was taken up in EtOH and a large excess of EtOAc was added slowly. The white precipitate was filtered and purified by preparative HPLC (H₂O/Acetonitrile/formic acid, 95:5:0.1 to 40:60:0.1) to give the clickable LCC-12 di-formic acid salt as a white powder (102 mg, 30%). ¹H-NMR (500 MHz, DMSO-d₆) δ=9.05-7.60 (brs, 4H, ex), 8.47 (s, 2H, formate), 7.60-6.80 (m, 8H, ex), 3.29-3.16 (m, 4H), 3.06 (brs, 4H), 2.84 (s, 2H), 2.39-2.29 (m, 4H), 1.44 (brs, 4H), 1.25 (brs, 16H) ppm. ¹³C-NMR (125.8 MHz, DMSO-d₆) δ=167.6 (formate), 159.7, 158.4, 82.6, 72.7, 41.3, 40.3, 29.5 (3C), 29.2, 26.8, 19.4 ppm. HRMS (ESI+) m/z: calculated for C₂₄H₄₆N₁₀ [M+2H]²⁺ 237.1948, found 237.1947. Isotopically labelled lipophilic copper clamp: Dicyandiamide ¹⁵N and ¹³C marked (Eurisotop, CNLM-9324-PK, 50 mg, 0.55 mmol), 1,12-diaminododecane (46.8 mg, 0.23 mmol) and CuCl₂ (31.4 mg, 0.23 mmol) were suspended in a sealed tube in water (0.6 mL) and stirred for 1 h, then heated at 80° C. for 48 h. The resulting pink mixture was acidified with an aq. solution of HCl (2 M, 1 mL) until complete dissolution of the precipitate. The mixture was concentrated under reduced pressure and isotopically labeled LCC-12 was purified by preparative HPLC (H₂O/Acetonitrile/formic acid, 95:5:0.1 to 73:27:0.1) to give the isotopically labeled LCC-12 di-formic acid salt as a white powder (35 mg, 32%). ¹H-NMR (500 MHz, DMSO-d₆) δ: 8.60-7.76 (brs, 2H, ex), 8.48 (s, 2H, formate), 7.70-6.50 (brs, 12H, ex), 3.05 (brs, 4H), 1.43 (m, 4H), 1.33-1.18 (m, 16H) ppm. ¹³C-NMR (125.8 MHz, DMSO-d₆) δ: 167.0 (formate), 160.2, 159.2, 41.3, 29.5 (3C), 29.2, 26.8 ppm.

Fluorescence Microscopy

Isolated monocytes were plated on cover slips, differentiated and activated as described in Cell culture. Cells were washed three times with 1×PBS, fixed with 2% paraformaldehyde in 1×PBS for 12 min and then washed three times with 1×PBS. After fixation, cells were permeabilized with 0.1% Triton X-100 in 1×PBS for 5 min and washed three times with 1×PBS. Subsequently, cells were blocked in 2% BSA, 0.2% Tween-20/1×PBS (blocking buffer) for 20 min at room temperature. Cells were incubated with the relevant antibody in blocking buffer for 1 h at room temperature, washed three times with 1×PBS and were incubated with secondary antibodies for 1 h. Finally, cover slips were washed three times with 1×PBS and mounted using VECTASHIELD containing DAPI (Vector Laboratories, H-1200-10). Fluorescence images were acquired using a Deltavision real-time microscope (Applied Precision). 40×/1.4NA, 60×/1.4NA and 100×/1.4NA objectives were used for acquisitions and all images were acquired as z-stacks. Images were deconvoluted with SoftWorx (Ratio conservative—15 iterations, Applied Precision) and processed with ImageJ.

Click labeling. aMDM on coverslips were treated with clickable LCC-12 (in-house, 0.1 μM, 3 h) in the absence or presence of CCCP (10 μM, 3 h) fixed and permeabilized as indicated in fluorescence microscopy. Mitotracker was added to live cells for 45 mins to before fixation. The click reaction cocktail was prepared using the Click-iT EdU Imaging kit (Life Technologies, C10337) according to the manufacturer's protocol. Briefly, we mixed 430 μL of 1× Click-iT reaction buffer with 20 μL of CuSO₄ solution, 1.2 μL Alexa-Fluor-azide, 50 μL reaction buffer additive (sodium ascorbate) to reach a final volume of ˜500 μL. Reactions were performed with or without CuSO₄ or ascorbate. Cover-slips were incubated with the click reaction cocktail in the dark at room temperature for 30 min, then washed three times with 1×PBS. Immunofluorescence was then performed as described in fluorescence microscopy.

1-17. (canceled)

18. A method of treating an inflammatory disease or disorder or an autoimmune disease or disorder comprising administering a compound of formula (I) to a subject having said inflammatory or autoimmune disease or disorder,
wherein the formula (I) is 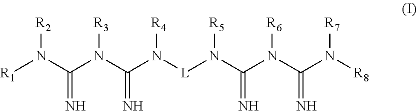
with
R₁, R₂, R₃, R₄, R₅, R₆, R₇ and R₈ being independently selected from the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group; or
R₂, R₃, R₄, R₅, R₆, and R₇ being independently selected from the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group, and R₁ and R₈ forming together a linker -L′-,
-L- and -L′-, if present, being independently a linear hydrocarbon chain of 8 to 16 carbons optionally interrupted by
an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;
with R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and
R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy,
or a pharmaceutically acceptable salt, stereoisomer, tautomer or solvate thereof.

19. The method according to claim 18, wherein said inflammatory disease or disorder or autoimmune disease or disorder is a systemic inflammatory response syndrome, a cytokine release syndrome (CRS), an Adult Respiratory Distress Syndrome (ARDS), a Macrophage Activation Syndrome (MAS), an Alveolar inflammatory response, a paediatric multisystem inflammatory syndrome, a Hemophagocytic lymphohistiocytosis (HLH), systemic lupus erythematosus, a sepsis, septic shock, Crohn's disease, ulcerative colitis, rheumatoid arthritis, or a hypercytokinemia.

20. The method according to claim 19, wherein the CRS is induced by a virus of Orthocoronavirinae subfamily.

21. The method according to claim 20, wherein said virus is Middle East respiratory syndrome-related coronavirus (MERS-CoV), β-CoV, Severe acute respiratory syndrome coronavirus (SARS-CoV), β-CoV or Severe acute respiratory syndrome coronavirus 2 (SARS-CoV-2), β-CoV.

22. The method according to claim 18, wherein said inflammatory disease or disorder or autoimmune disease or disorder is selected from the group consisting of a metabolic disease, diabetes mellitus, type 1 diabetes mellitus, type 2 diabetes mellitus, insulin resistance, hyperglycemia, hyperinsulinemia, glucose intolerance, hypertension, NAFLD, NASH and obesity, polycystic ovary syndrome, metabolic syndrome, cardiovascular diseases, hypertension, atherosclerosis and arteriosclerosis, a mitochondrial dysfunction, a primary or a secondary mitochondrial dysfunction, a secondary mitochondrial disorder due to copper overload, Indian childhood cirrhosis, Wilson's disease, Idiopathic infantile copper toxicosis due to iron overload, Hereditary hemochromatosis, Juvenile Hemochromatosis, Neonatal iron storage disease, type I Tyrosinemia, Zellweger syndrome, mental disorders, schizophrenia, anxiety disorders, mild cognitive disorder, depressive disorder, bipolar disorder, autism spectrum disorder, Fragile X syndrome, an infection by a virus, a neurodegenerative disease or disorder and aging.

23. The method according to claim 22, wherein said infection by a virus is a infection by a coronavirus or an influenza virus.

24. The method according to claim 22, wherein said inflammatory disease or disorder or autoimmune disease or disorder is selected from the group consisting of diabetes mellitus, type 1 diabetes mellitus, type 2 diabetes mellitus, polycystic ovary syndrome, and glucose intolerance.

25. The method according to claim 18, wherein the compound is selected from the group consisting of 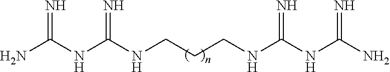
with n being an integer from 6 to 14; 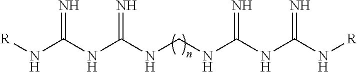
with n being an integer from 8 to 16, and R being a C₁-C₆alkyl, or a C₀-C₃alkyl-ethynyl (—(CH₂)₀₋₃—C≡CH); 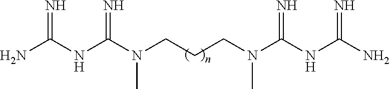
with n being an integer from 6 to 14; 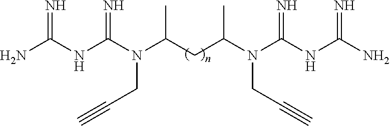
with n being an integer from 6 to 14; 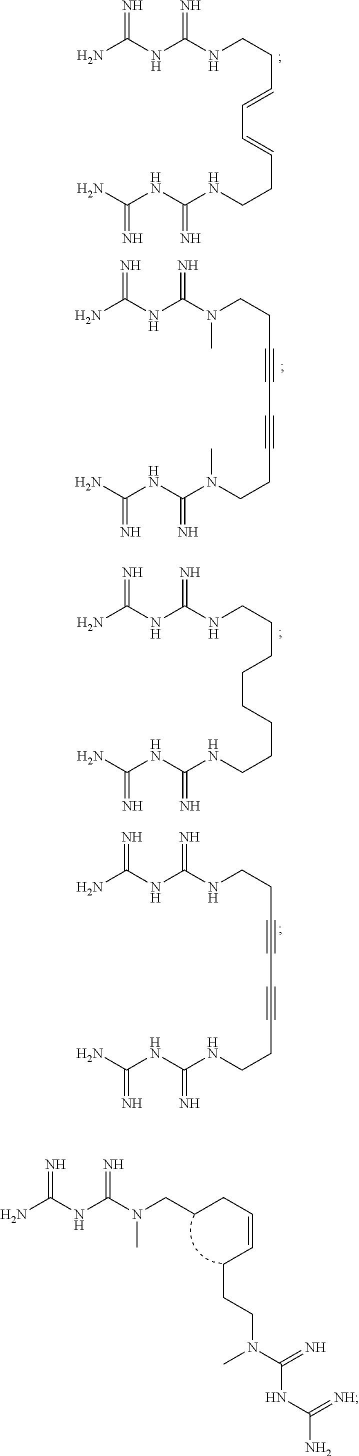 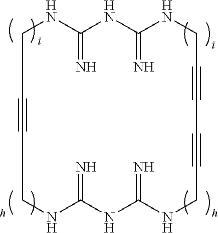
with “h” and “i” being an integer independently selected from 0 to 14 and the sum “h” and “i” being an integer selected from 4 to 14, or from 4 to 12, or from 5 to 10, or from 6 to 10; and 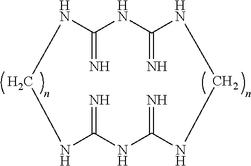
with “n” being an integer selected from 8 to 16; wherein the dotted line being present or absent and being one or two atoms with covalent bonds,
or a pharmaceutically acceptable salt, stereoisomer, tautomer or solvate thereof.

26. The method according to claim 18, wherein R₁ and R₈ are independently selected from the group consisting of H, a C₁-C₆alkyl and a C₀-C₃alkyl-ethynyl (—(CH₂)₀₋₃—C≡CH), or they form together a linker -L′-.

27. The method according to claim 18, wherein R₂, R₃, R₄, R₅, R₆, and R₇ are independently selected from the group consisting of H, a C₁-C₆alkyl and a C₀-C₃alkyl-ethynyl (—(CH₂)₀₋₃—C≡CH).

28. The method according to claim 18, wherein R₃ and R₆ are H.

29. The method according to claim 18, wherein R₂ and R₇ are H.

30. The method according to claim 18, wherein L, and L′ if present, is independently
—(CH₂)_{f}—CRₐ═CRₑ—CR_{f}═CR_{b}—(CH₂)_{g}—, —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 0 to 12, or from 3 to 10, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 0 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 0 to 14, or from 3 to 12, or from 4 to 10, or from 5 to 10, or from 6 to 10; or
—(CH₂)ₙ—, with “n” being an integer selected from 4 to 16; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), and with “n” being an integer selected from 2 to 14; or
—(CH₂)ₚ—CHR_{c}—CRₑ═CR_{f}—CHR_{d}—(CH₂)_{q}—, —(CH₂)ₚ—CHRₑ—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 0 to 12, or from 3 to 10, or from 4 to 10, or from 5 to 10, or from 6 to 10.

31. The method according to claim 18, wherein L, and L′ if present, is independently
—(CH₂)_{f}—CRₐ═CRₑ—CR_{f}═CR_{b}—(CH₂)_{g}—, —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 4 to 12, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 4 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 6 to 14, or from 6 to 10; or
—(CH₂)ₙ—, with “n” being an integer selected from 8 to 16; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), and with “n” being an integer selected from 6 to 14; or
—(CH₂)ₚ—CHR_{c}—CRₑ═CR_{f}—CHR_{d}—(CH₂)_{q}—, or —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 4 to 12, or from 4 to 10, or from 5 to 10, or from 6 to 10.

32. The method according to claim 18, wherein L, and L′ if present, is independently
—(CH₂)_{f}—CRₐ═CH—CH═CR_{b}—(CH₂)_{g}—, with Rₐ and R_{b} being H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 7 to 12, or from 7 to 11, or from 8 to 11, or from 9 to 10; or
—(CH₂)ₕ—C≡C—C≡C—(CH₂)ᵢ—, with “h” and “i” being an integer independently selected from 0 to 12 and the sum “h” and “i” being an integer selected from 7 to 12, or from 7 to 11, or from 8 to 11, or from 9 to 10; or
—(CH₂)ₕ—C≡C—(CH₂)ᵢ—, with “h” and “i” being an integer independently selected from 0 to 14 and the sum “h” and “i” being an integer selected from 9 to 14, or from 10 to 13, or from 10 to 12; or
—(CH₂)ₙ—, with “n” being an integer selected from 11 to 16; or
—CRₐ—(CH₂)ₙ—CR_{b}—, with Rₐ and R_{b} being H, a C₁-C₆alkyl or a C₀-C₃alkyl-ethynyl (—(CH₂)₀₋₆—C≡CH), and with “n” being an integer selected from 9 to 14; or
—(CH₂)ₚ—CHR_{c}—CH═CH—CHRₐ—(CH₂)_{q}—, with R_{c} and R_{d} being H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 7 to 12, or from 7 to 11, or from 8 to 11, or from 9 to 10.

33. The method according to claim 18, wherein the compound is selected from the group consisting of 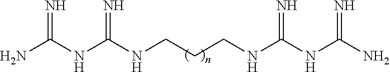
with n being an integer from 9 to 14; 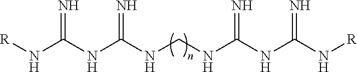
with n being an integer from 11 to 16 and R being a C₁-C₆alkyl, or a C₀-C₃alkyl-ethynyl (—(CH₂)₀₋₃—C≡CH); 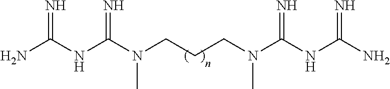
with n being an integer from 9 to 14; 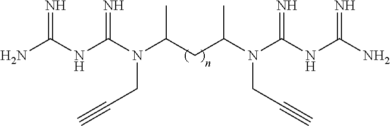
with n being an integer from 9 to 14; 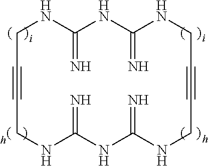
with “h” and “i” being an integer independently selected from 0 to 14 and the sum “h” and “i” being an integer selected from 4 to 14, or from 4 to 12, or from 5 to 10, or from 6 to 10; and 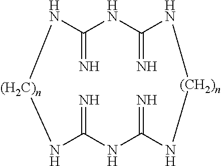
with “n” being an integer selected from 8 to 16; 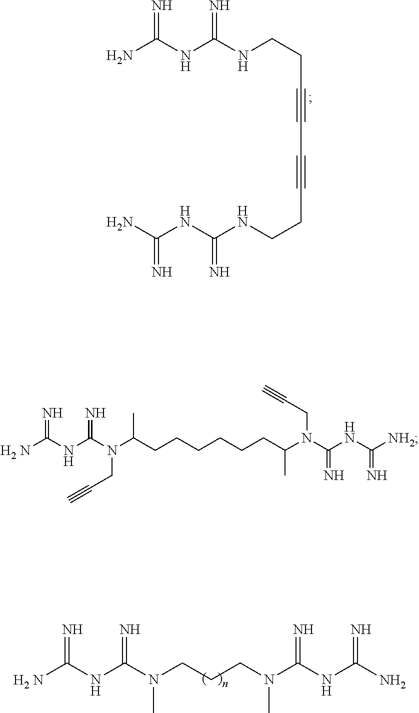
with n being an integer from 2-14 or from 4-14 or from 8-14 or from 8-12 or from 8-10; and 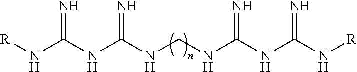
with n being an integer from 6-16, from 8-16 or from 8-12 or from 8-10 and R being an ethyl, a propyl or —CH2-C≡CH,
or a pharmaceutically acceptable salt, stereoisomer, tautomer or solvate thereof.

34. A compound of formula (I) wherein the formula (I) is 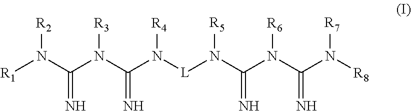
wherein
R₂, R₃, R₄, R₅, R₆, and R₇ being independently selected from the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group;
and
1) R₁ and R₈ being independently selected from the group consisting of H, a C₁-C₆alkyl, a C₀-C₆alkyl-ethynyl (—(CH₂)₀₋₆—C≡CH), a C₃-C₁₀cycloalkyl, a C₃-C₁₀cycloheteroalkyl, a C₆-C₁₂aryl, and a C₅-C₁₂heteroaryl, said alkyl, cycloalkyl, cycloheteroalkyl, aryl or heteroaryl being optionally substituted by a R group or a R′ group; and
-L- being independently a linear hydrocarbon chain of 11 to 16 carbons optionally interrupted by
an oxygen atom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;
or
2) R₁ and R₈ forming together a linker -L′-; and
-L- and -L′- being independently a linear hydrocarbon chain of 4 to 16 carbons optionally interrupted by
a heteroatom; and/or
a function selected from the group consisting of amide (—C(O)—NH— or —NH—C(O)—), carbonyl (—C(O)—), ester (—C(O)—O— or —O—C(O)—), sulfonyl (—SO₂—), sulfinyl (—S(O)—), thiocarbonyl (—C(S)—), thioester (—C(O)—S— or —S—C(O)—), carbonyloxy (—O—C(O)—O—), —S(O)—NH—, —NH—S(O)—, —SO₂—NH—, —NH—SO₂—, phosphate (—O—P(O)OH—O—) and phosphonate (—P(O)OH—O— or —O—P(O)OH—); and/or
a 3-7-membered ring, optionally selected from the group consisting of a cycloalkyl, a heterocycloalkyl, an aryl, and a heteroaryl, said ring being optionally substituted by a group R or R′;
said linear hydrocarbon chain being optionally substituted by a group R or R′;
or
3) the compound is selected from the group consisting of 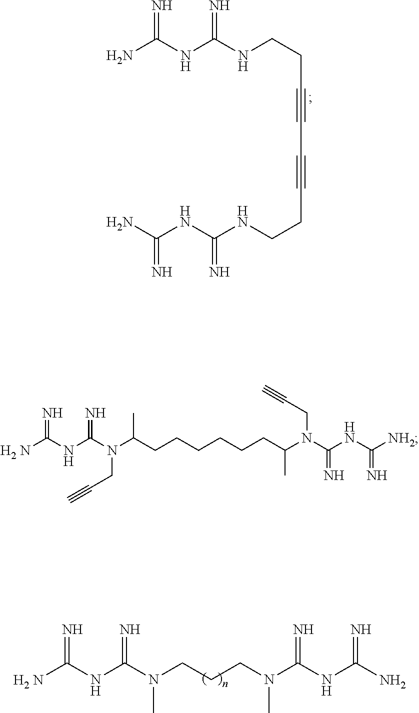
with n being an integer from 2-14 or from 4-14 or from 8-14 or from 8-12 or from 8-10; and 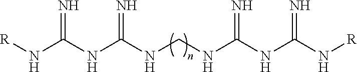
with n being an integer from 6-16, from 8-16 or from 8-12 or from 8-10 and R being an ethyl, a propyl or —CH₂—C≡CH,
wherein in item 1) and 2)
R being selected from the group consisting of a C₁-C₆alkyl optionally substituted by at least one halogen, a C₁-C₆alkyloxy optionally substituted by at least one halogen, a C₃-C₁₀cycloalkyl, a C₃-C₁₀cycloheteroalkyl, a C₆-C₁₂aryl, and a C₅-C₁₂heteroaryl, the group being optionally substituted by a group R′, and
R′ being selected from the group consisting of a halogen, a hydroxyl, a thiol, a cyano, an ethynyl (—C≡CH), a nitro, an amino (—NH₂), a phosphate (PO₄³⁻), a C₁-C₆alkyl optionally substituted by at least one halogen, a C₁-C₆alkoxy optionally substituted by at least one halogen, a C₁-C₆thioalkyl optionally substituted by at least one halogen, C₀-C₃alkyl-NH—C(O)—R″, C₀-C₃alkyl-C(O)—NR″R″, C₀-C₃alkyl-NH—C(O)—OR″, C₀-C₃alkyl-NH—C(O)—NR″R″, C₀-C₃alkyl-C(O)—R″, C₀-C₃alkyl-C(O)—OR″, C₀-C₃alkyl-NR″R″, C₀-C₃alkyl-SOR″, C₀-C₃alkyl-SO₂R″, C₀-C₃alkyl-SONR″R″, C₀-C₃alkyl-SO₂NR″R″, C₀-C₃alkyl-NHSO₂R″, -a C₃-C₁₀cycloalkyl, a C₃-C₁₀cycloheteroalkyl, a C₆-C₁₂aryl, and a C₅-C₁₂heteroaryl, with R″ being H or a C₁-C₃ or C₁-C₆alkyl optionally substituted by at least one halogen and said cycloalkyl, cycloheteroalkyl, aryl or heteroaryl being optionally substituted by a halogen, a hydroxyl, a cyano, a nitro, an amino, or a C₁-C₃alkoxy,
or a pharmaceutically acceptable salt, stereoisomer, tautomer or solvate of any of these compounds.

35. The compound according to claim 34, wherein R₁ and R₈ are independently selected from the group consisting of H, a C₁-C₆alkyl and a C₀-C₃alkyl-ethynyl (—(CH₂)₀₋₃—C≡CH), or they form together a linker -L′-.

36. The compound according to claim 34, wherein R₂, R₃, R₄, R₅, R₆, and R₇ are independently selected from the group consisting of H, a C₁-C₆alkyl and a C₀-C₃alkyl-ethynyl (—(CH₂)₀₋₃—C≡CH).

37. The compound according to claim 34, wherein R₃ and R₇ are H.

38. The compound according to claim 34, wherein R₂ and R₇ are H.

39. The compound according to claim 34, wherein L, and L′ if present, is independently
—(CH₂)_{f}—CRₐ═CRₑ—CR_{f}═CR_{b}—(CH₂)_{g}—, —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 0 to 12, or from 3 to 10, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 0 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 0 to 14, or from 3 to 12, or from 4 to 10, or from 5 to 10, or from 6 to 10; or
—(CH₂)ₙ—, with “n” being an integer selected from 4 to 16; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), and with “n” being an integer selected from 2 to 14; or
—(CH₂)ₚ—CHR_{c}—CRₑ═CR_{f}—CHR_{d}—(CH₂)_{q}—, —(CH₂)ₚ—CHRₑ—CH═CH—CHR_{d}—(CH₂)_{q}—, with Rₑ and R_{d} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 0 to 12, or from 3 to 10, or from 4 to 10, or from 5 to 10, or from 6 to 10.

40. The compound according to claim 34, wherein L, and L′ if present, is independently
—(CH₂)_{f}—CRₐ═CRₑ—CR_{f}═CR_{b}—(CH₂)_{g}—, —(CH₂)_{f}—CRₐ═CH—CH═CR_{b}—(CH₂)_{g}—, with Rₐ and R_{b} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 4 to 12, or from 4 to 10, or from 5 to 10, or from 6 to 10; or
—(CH₂)ₕ—C≡C—C≡C—(CH₂)ᵢ—, with “h” and “i” being an integer independently selected from 0 to 12 and the sum “h” and “i” being an integer selected from 4 to 12, or from 3 to 10, or from 4 to 10, or from 5 to 10, or from 6 to 10; or
—(CH₂)ₕ—C≡C—(CH₂)ᵢ—, with “h” and “i” being an integer independently selected from 0 to 14 and the sum “h” and “i” being an integer selected from 6 to 14, or from 6 to 10; or
—(CH₂)ₙ—, with “n” being an integer selected from 8 to 16; or
—CRₐ—(CH₂)ₙ—CR_{b}—, with Rₐ and R_{b} being independently H, a C₁-C₆alkyl or a C₀-C₃alkyl-ethynyl (—(CH₂)₀₋₆—C≡CH), and with “n” being an integer selected from 6 to 14; or
—(CH₂)ₚ—CHR_{c}—CRₑ═CR_{f}—CHR_{d}—(CH₂)_{q}—, or —(CH₂)ₚ—CHR_{c}—CH═CH—CHR_{d}—(CH₂)_{q}—, with R_{c} and R_{d} being independently H, a C₁-C₆alkyl or a C₀-C₃alkyl-ethynyl (—(CH₂)₀₋₆—C≡CH), with Rₑ and R_{f} being independently H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 4 to 12, or from 4 to 10, or from 5 to 10, or from 6 to 10.

41. The compound according to claim 34, wherein L, and L′ if present, is independently
—(CH₂)_{f}—CRₐ═CH—CH═CR_{b}—(CH₂)_{g}—, with Rₐ and R_{b} being H, a C₁-C₆alkyl or a C₀-C₃alkyl-ethynyl (—(CH₂)₀₋₆—C≡CH), “f” and “g” being an integer independently selected from 0 to 12 and the sum “f” and “g” being an integer selected from 7 to 12, or from 7 to 11, or from 8 to 11, or from 9 to 10; or
—(CH₂)ₕ—C≡C—C≡C—(CH₂)ᵢ—, with “h” and “i” being an integer independently selected from 0 to 12 and the sum “h” and “i” being an integer selected from 7 to 12, or from 7 to 11, or from 8 to 11, or from 9 to 10; or
—(CH₂)ₕ—C≡C—(CH₂)ᵢ—, with “h” and “i” being an integer independently selected from 0 to 14 and the sum “h” and “i” being an integer selected from 9 to 14, or from 10 to 13, or from 10 to 12; or
—(CH₂)ₙ—, with “n” being an integer selected from 11 to 16; or
—CRₐ—(CH₂)ₙ—CR_{b}—, with Rₐ and R_{b} being H, a C₁-C₆alkyl or a C₀-C₃alkyl-ethynyl (—(CH₂)₀₋₆—C≡CH), and with “n” being an integer selected from 9 to 14; or
—(CH₂)ₚ—CHR_{c}—CH═CH—CHRₐ—(CH₂)_{q}—, with R_{c} and R_{d} being H, a C₁-C₆alkyl or a C₀-C₃alkyl-ethynyl (—(CH₂)₀₋₆—C≡CH), “p” and “q” being an integer independently selected from 0 to 12 and the sum “p” and “q” being an integer selected from 7 to 12, or from 7 to 11, or from 8 to 11, or from 9 to 10.

42. The compound according to claim 34, wherein the compound is selected from the group consisting of 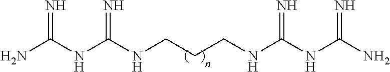
with n being an integer from 9 to 14; 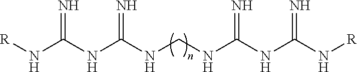
with n being an integer from 11 to 16 and R being a C₁-C₆alkyl, or a C₀-C₃alkyl-ethynyl (—(CH₂)₀₋₃—C≡CH); 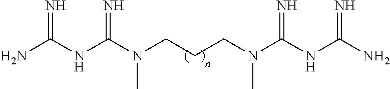
with n being an integer from 9 to 14; 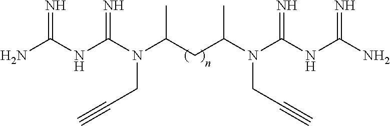
with n being an integer from 9 to 14; 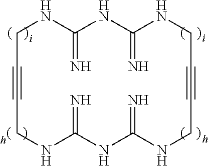
with “h” and “i” being an integer independently selected from 0 to 14 and the sum “h” and “i” being an integer selected from 4 to 14, or from 4 to 12, or from 5 to 10, or from 6 to 10; and 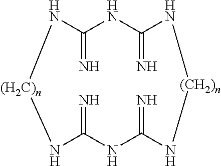
with “n” being an integer selected from 8 to 16; 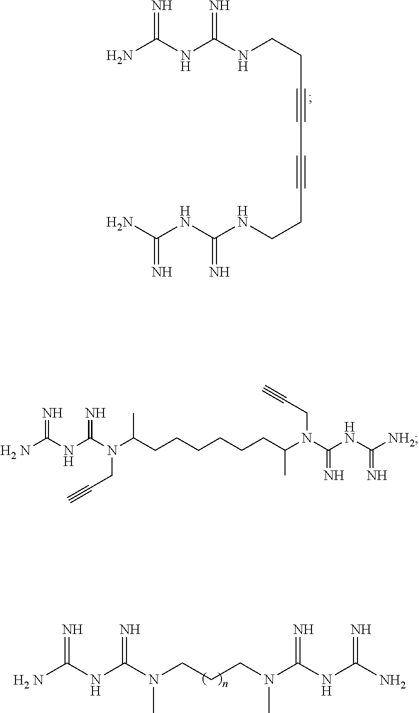
with n being an integer from 2-14 or from 4-14 or from 8-14 or from 8-12 or from 8-10; and 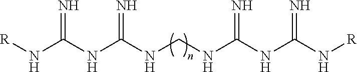
with n being an integer from 6-16, from 8-16 or from 8-12 or from 8-10 and R being an ethyl, a propyl or —CH2-C≡CH,
or a pharmaceutically acceptable salt, stereoisomer, tautomer or solvate thereof.

43. A method of treating a disease comprising administering a compound according to claim 34 to a subject in need of treatment, said disease being selected from the group consisting of a cancer, a metabolic disease, diabetes mellitus, type 1 diabetes mellitus, type 2 diabetes mellitus, insulin resistance, hyperglycemia, hyperinsulinemia, glucose intolerance, hypertension, NAFLD, NASH and obesity, polycystic ovary syndrome, metabolic syndrome, cardiovascular diseases, hypertension, atherosclerosis and arteriosclerosis, a secondary mitochondrial disorder due to copper overload, Indian childhood cirrhosis, Wilson's disease, Idiopathic infantile copper toxicosis due to iron overload, Hereditary hemochromatosis, Juvenile Hemochromatosis, Neonatal iron storage disease, type I Tyrosinemia, Zellweger syndrome, mental disorders, schizophrenia, anxiety disorders, mild cognitive disorder, depressive disorder, bipolar disorder, autism spectrum disorder, Fragile X syndrome, an infection by a virus, a neurodegenerative disease or disorder and aging.

44. The method according to claim 43, wherein said infection by a virus is a infection by a coronavirus or an influenza virus.

45. The method according to claim 43, wherein said inflammatory disease or disorder or autoimmune disease or disorder is selected from the group consisting of diabetes mellitus, type 1 diabetes mellitus, type 2 diabetes mellitus, polycystic ovary syndrome, and glucose intolerance.
